(19) United States
(12) Patent Application Publication
(10) Pub. No.: US 2024/0325535 A1
(43) Pub. Date: Oct. 3, 2024

(54) COMPOSITIONS AND METHODS COMPRISING CAR T CELLS COMPRISING PRDM1 AND/OR NR4A3 KNOCKOUT

(71) Applicant: THE TRUSTEES OF THE UNIVERSITY OF PENNSYLVANIA, Philadelphia, PA (US)

(72) Inventors: Joseph A. Fraietta, Voorhees, NJ (US); Inyoung Jung, Philadelphia, PA (US); Naomi Balzer-Haas, Haverford, PA (US); Vivek Narayan, Swarthmore, PA (US)

(21) Appl. No.: 18/708,529

(22) PCT Filed: Nov. 10, 2022
(86) PCT No.: PCT/US2022/079634

§ 371 Date: May 8, 2024

Related U.S. Application Data
(60) Provisional application No. 63/278,370, filed on Nov. 11, 2021.

(51) Int. Cl. A61K 39/00; A61P 35/00; C07K 14/705; C07K 14/725; C07K 16/30

(52) U.S. Cl. CPC A61K 39/4644; A61K 39/4611; A61K 39/4631; A61P 35/00; C07K 14/70503; C07K 14/7051; C07K 14/70521; C07K 14/70532; C07K 14/70578; C07K 14/70596; C07K 16/30; C07K 2319/03

(57) ABSTRACT

The present disclosure provides modified immune cells or precursors thereof (e.g., gene edited modified T cells) comprising a chimeric antigen receptor (CAR) and wherein PRDM1 and/or NR4A3 and/or PRDM1 are knocked-out. Compositions and methods of treatment are also provided.

### CROSS-REFERENCE TO RELATED APPLICATION

The present application is a 35 U.S.C. § 371 national phase application of, and claims priority to, International Application No. PCT/US2022/079634, filed Nov. 10, 2022, which claims priority under 35 U.S.C. § 119(e) to U.S. Provisional Patent Application No. 63/278,370, filed Nov. 11, 2021, the contents of each of which are hereby incorporated by reference in their entireties.

### REFERENCE TO SEQUENCE LISTING

The Sequence Listing concurrently submitted herewith as a text file named “046483-7352US1_sequence_listing.xml” created on May 3, 2024, and having a size of 30,805 bytes is herein incorporated by reference in its entirety pursuant to 37 C.F.R. 1.52(e)(5).

### BACKGROUND OF THE INVENTION

Chimeric antigen receptor (CAR) T-cells have induced unprecedented high rates of complete remission in relapsed and refractory B-cell malignancies. Despite these successes, a substantial portion of patients with B-cell leukemia, lymphoma, and myeloma fail to respond to CAR T-cell therapy and only a small subset of patients achieve long-term durable responses. In any of the B-cell cancers, complete responses are associated with robust CAR T-cell proliferation, with a distinct advantage of long-term CAR T-cell persistence. In this regard, T cell-intrinsic negative regulatory mechanisms, such as upregulation of naturally occurring negative immune checkpoint molecules and the attrition of stem cell memory/central memory functions are major barriers to the success of CAR T-cell therapy. Further, the efficacy of CAR T-cell therapy in solid tumor indications has been limited to date. Unlike the situation in hematologic malignancies, CAR T-cells must traffic to solid tumor sites and surmount stromal elements to infiltrate into the tumor bed and elicit antigen-directed cytotoxicity. Even if trafficking and infiltration are successful, CAR T-cells often become dysfunctional due to chronic antigen exposure and additional immunosuppressive mechanisms operative within the tumor microenvironment (TME).

CAR T-cells derived from naïve and early memory subsets have been shown to robustly expand in vivo and are long-lived with a self-renewal capacity. Naïve or early memory T-cells genetically redirected with CARs have more durable engraftment and antitumor effector function compared to highly differentiated cells. However, persistent tumor antigen exposure in the setting of hematopoietic and non-hematopoietic cancers often leads to exhaustion. T-cell exhaustion is characterized by upregulation of multiple inhibitory receptors, the inability to respond to homeostatic cytokines, loss of effector function and reduced survival. CAR T-cell exhaustion can also be facilitated by antigen-independent tonic signaling through the synthetic antigen receptor during cell manufacturing or following infusion.

Clinical resistance to CAR T-cell therapy has been attributed to the failure of expansion, engraftment or durability of the antitumor response following adoptive cell transfer. Sustained remission was associated with an increased peak expansion of chronic lymphocytic leukemia (CLL) patient anti-CD19 CAR T-cells after infusion and relatively longer persistence. Cell products that were particularly effective showed greater proliferative capacity prior to and during treatment. Transcriptomic analysis suggested that remission correlated with early memory T-cell signatures as well as sternness, while gene expression profiles from non-responders were associated with terminal differentiation and exhaustion. Similarly, memory CD8⁺ T-cell transcriptional programs enriched in axicabtagene ciloleucel infusion products were strongly related to the long-term durability of response, whereas gene signatures of exhaustion correlated with early treatment failure in patients with large B-cell lymphomas (LBCL). Importantly, it may be possible to overcome many of the above issues by using highly proliferative, ‘optimally programmed’ CAR T-cells.

More recently, extensive profiling of apheresed T-cells from ALL patients using bulk and single-cell RNA sequencing identified that the Transcription Factor 7 (TCF7) network, which plays a central role in preserving naïve and early memory T-cell states, is maintained in effector T-cells in patients with long-term CAR T-cell survival. Conversely, an upregulated interferon (IFN) signaling profile was associated with poor CAR T-cell persistence. These studies have thus elucidated some of the molecular underpinnings and T-cell states associated with response and resistance to CAR T-cell therapies in advanced B-cell malignancies. However, the cellular basis for resistance to CAR T-cell therapy in solid tumors has not been elucidated and there are limited engineering approaches to generate CAR T-cells with desirable features identified from correlative analyses.

There is a need in the art for improved CAR T cell therapies. The present disclosure addresses this need.

### SUMMARY OF THE INVENTION

In one aspect, the invention provides a modified immune cell or precursor cell thereof, comprising: a modification in an endogenous gene locus encoding PRDM1, wherein the modification is capable of downregulating gene expression of, or knocking out, endogenous PRDM1; a modification in an endogenous gene locus encoding NR4A3, wherein the modification is capable of downregulating gene expression of, or knocking out, endogenous NR4A3; and an exogenous T cell receptor (TCR) and/or chimeric antigen receptor (CAR) comprising affinity for an antigen on a target cell.

In another aspect, the invention provides a modified immune cell or precursor cell thereof, comprising: a modification in an endogenous gene locus encoding PRDM1, wherein the modification is capable of downregulating gene expression of, or knocking out, endogenous PRDM1; and an exogenous T cell receptor (TCR) and/or chimeric antigen receptor (CAR) comprising affinity for an antigen on a target cell.

In another aspect, the invention provides a modified immune cell or precursor cell thereof, comprising: a modification in an endogenous gene locus encoding PRDM1, wherein the modification is capable of downregulating gene expression of, or knocking out, endogenous PRDM1; a modification in an endogenous gene locus encoding TGFβRII, wherein the modification is capable of downregulating gene expression of, or knocking out, endogenous TGFβRII; and an exogenous T cell receptor (TCR) and/or chimeric antigen receptor (CAR) comprising affinity for an antigen on a target cell.

In various embodiments of the above aspects or any other aspect of the invention delineated herein, the modification comprises a CRISPR-mediated modification. In certain embodiments, the CRISPR-mediated modification is introduced by a CRISPR system comprising a guide RNA that comprises a guide sequence that is sufficiently complementary with a target sequence in the endogenous gene locus encoding PRDM1, NR4A3 or TGFβRII. In certain embodiments, the guide RNA comprises a nucleic acid sequence set forth in SEQ ID NO: 2 or SEQ ID NO: 4. In certain embodiments, the modification is selected from the group consisting of a substitution, an insertion, a deletion, and an insertion/deletion.

In certain embodiments, the CAR comprises an antigen binding domain, a transmembrane domain, and an intracellular domain. In certain embodiments, the antigen binding domain is capable of binding a tumor associated antigen (TAA). In certain embodiments, the antigen binding domain is selected from the group consisting of an antibody, an scFv, and a Fab.

In certain embodiments, the transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

In certain embodiments, the CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

In certain embodiments, the CAR comprises an intracellular domain comprising an intracellular domain selected from the group consisting of cytoplasmic signaling domains of a human CD3 zeta chain, FcγRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

In certain embodiments, the antigen on a target cell is a tumor associated antigen (TAA).

In certain embodiments, the modified cell is resistant to cell exhaustion. In certain embodiments, the modified cell is an autologous cell. In certain embodiments, the modified cell is a cell isolated from a human subject. In certain embodiments, the modified cell is a modified immune cell. In certain embodiments, the modified cell is a modified T cell. In certain embodiments, the modified cell is a gamma delta T cell. In certain embodiments, the modified cell is a modified T cell resistant to T cell exhaustion.

Another aspect of the invention includes a method of treating cancer in a subject in need thereof. The method comprises administering to the subject a composition comprising any of the modified immune cells or precursor cells thereof contemplated herein.

Another aspect of the invention includes a method for generating a modified immune cell or precursor cell thereof. The method comprises introducing into an immune or precursor cell a CRISPR system comprising one or more polypeptides and/or nucleic acids capable of downregulating gene expression of endogenous PRDM1; and introducing into the immune or precursor cell a nucleic acid encoding an exogenous CAR and/or TCR, wherein the exogenous CAR and/or TCR comprises affinity for an antigen on a target cell.

Another aspect of the invention includes a method for generating a modified immune cell or precursor cell thereof, comprising: introducing into an immune or precursor cell a CRISPR system comprising one or more polypeptides and/or nucleic acids capable of downregulating gene expression of endogenous PRDM1; introducing into an immune or precursor cell a CRISPR system comprising one or more polypeptides and/or nucleic acids capable of downregulating gene expression of endogenous NR4A3; and introducing into the immune or precursor cell a nucleic acid encoding an exogenous CAR and/or TCR, wherein the exogenous CAR and/or TCR comprises affinity for an antigen on a target cell.

Another aspect of the invention includes a method for generating a modified immune cell or precursor cell thereof, comprising: introducing into an immune or precursor cell a CRISPR system comprising one or more polypeptides and/or nucleic acids capable of downregulating gene expression of endogenous PRDM1; introducing into an immune or precursor cell a CRISPR system comprising one or more polypeptides and/or nucleic acids capable of downregulating gene expression of endogenous TGFβRII; and introducing into the immune or precursor cell a nucleic acid encoding an exogenous CAR and/or TCR, wherein the exogenous CAR and/or TCR comprises affinity for an antigen on a target cell.

In certain embodiments, the one or more polypeptides and/or nucleic acids capable of downregulating gene expression of endogenous PRDM1 introduces a CRISPR-mediated modification in an endogenous gene locus encoding PRDM1, and/or the one or more polypeptides and/or nucleic acids capable of downregulating gene expression of endogenous NR4A3 introduces a CRISPR-mediated modification in an endogenous gene locus encoding NR4A3, and/or the one or more polypeptides and/or nucleic acids capable of downregulating gene expression of endogenous TGFβRII introduces a CRISPR-mediated modification in an endogenous gene locus encoding TGFβRII.

In certain embodiments, the modification is selected from the group consisting of a substitution, an insertion, a deletion, and an insertion/deletion.

In certain embodiments, the CRISPR system comprises a CRISPR nuclease and a guide RNA. In certain embodiments, the CRISPR nuclease is Cas9. In certain embodiments, CRISPR nuclease and the guide RNA comprise a ribonucleoprotein (RNP) complex. In certain embodiments, the RNP complex is introduced by electroporation. In certain embodiments, the guide RNA comprises a nucleic acid sequence set forth in SEQ ID NO: 2 or SEQ ID NO: 4.

In certain embodiments, the nucleic acid encoding an exogenous CAR and/or TCR is introduced via viral transduction. In certain embodiments, the viral transduction comprises contacting the immune or precursor cell with a viral vector comprising the nucleic acid encoding an exogenous CAR and/or TCR. In certain embodiments, the viral vector is selected from the group consisting of a retroviral vector, a lentiviral vector, an adenoviral vector, and an adeno-associated viral vector. In certain embodiments, the viral vector is a lentiviral vector.

Another aspect of the invention includes a method of treating cancer in a subject in need thereof. The method comprises administering to the subject modified immune or precursor cell generated by any of the methods contemplated herein.

Another aspect of the invention includes a method of treating a disease or disorder in a subject in need thereof, comprising administering to the subject a modified T cell comprising: a modification in an endogenous gene locus encoding PRDM1, wherein the modification is capable of downregulating gene expression of, or knocking out, endogenous PRDM1; and an exogenous T cell receptor (TCR) and/or chimeric antigen receptor (CAR) comprising affinity for an antigen on a target cell.

Another aspect of the invention includes a method of treating a disease or disorder in a subject in need thereof, comprising administering to the subject a modified T cell comprising: a modification in an endogenous gene locus encoding PRDM1, wherein the modification is capable of downregulating gene expression of, or knocking out, endogenous PRDM1; a modification in an endogenous gene locus encoding NR4A3, wherein the modification is capable of downregulating gene expression of, or knocking out, endogenous NR4A3; and an exogenous T cell receptor (TCR) and/or chimeric antigen receptor (CAR) comprising affinity for an antigen on a target cell.

Another aspect of the invention includes a method of treating a disease or disorder in a subject in need thereof, comprising administering to the subject a modified T cell comprising: a modification in an endogenous gene locus encoding PRDM1, wherein the modification is capable of downregulating gene expression of, or knocking out, endogenous PRDM1; a modification in an endogenous gene locus encoding TGFβRII, wherein the modification is capable of downregulating gene expression of, or knocking out, endogenous TGFβRII; and an exogenous T cell receptor (TCR) and/or chimeric antigen receptor (CAR) comprising affinity for an antigen on a target cell.

In certain embodiments, the antigen on a target cell is a tumor associated antigen (TAA). In certain embodiments, the disease or disorder is cancer.

In certain embodiments, the modified T cell is a gamma delta T cell. In certain embodiments, the modified T cell is autologous. In certain embodiments, the subject is a human.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIGS. 1A-1J: TCF7+ CD8 and TIM3+ CD8 populations within the infusion product are associated with favorable and poor CAR T-cell therapeutic potency, respectively. FIG. 1A: Uniform manifold approximation and projection (UMAP) plot showing subclustering of CD8⁺ T-cells from prostate cancer patient CAR-T infusion products. Cells are labeled with marker gene expression and patient origin. FIGS. 1B-1D: Scores of gene signatures enriched in (FIG. 1C) TCF7⁺ T-cells in LCMV clone 13 (GSE83978; left) and LCMV Armstrong model (GSE83978; right), (FIG. 1D) exhausted T-cells (GSE136796), and (FIG. 1E) interferon response genes (dbGaP phs002323.v1.pl). FIGS. 1E-1F: Gene signature score enriched in (FIG. 1F) premanufacture T-cells from ALL patients with poor CD19 CAR T-cell persistence (PMID33820778), (FIG. 1G) anti-CD19 CAR T-cell infusion products of complete responder (CR) patients (GSE151511; left) and non-responding patients (GSE151511; right). FIG. 1H: Differentially expressed genes compared between TCF7⁺CD8⁺ and TIM3⁺CD8 clusters⁺. Top bars indicate cell clusters, patient origin, CD19 CAR T-cell response score (GSE151511), and cell cycle. FIG. 1I: Differential expression of transcription factors between TCF7⁺CD8⁺ and TIM3⁺CD8⁺ clusters. FIG. 1J: Expression levels of PRDM1 and TCF7 in CD8+ subclusters. FIG. 1B: PRDM1 and TCF7 expression levels in CD19 CAR-T infusion products from chronic lymphocytic leukemia patients (CR: complete response; PRTD: very good partial response; PR: partial response; NR: no response); (FPKM: Fragments per kilo base of transcript per million mapped fragments). *P<0.05, *P<0.01, ***P<0.001, n.s.: not significant (Kruskal-Wallis test with a post hoc Dunn's multiple comparison test).

FIGS. 2A-2M: CRISPR/Cas9-mediated PRDM1 KO potentiates early memory PSMA CAR T-cell differentiation of FIG. 2A: PRDM1 editing efficiency measured by TIDE (Tracking of Indels by Decomposition) analysis. FIG. 2B: Amplicon sequencing of PRDM1 indel variants generated by CRISPR/Cas9-mediated gene editing. FIG. 2C: Representative Western blot analysis for BLIMP1 expression. FIG. 2D: A schematic of the restimulation assay used to ‘stress test’ PRDM1 KO CAR T-cells. CAR T-cells were challenged with target cell PC3-PSMA every 4-5 days at a ratio of E:T=3:1. FIG. 2E: Effector cytokines produced by CAR T-cells after initial tumor cell challenge. FIG. 2F: Representative CAR T-cell expansion kinetics during restimulation assay from one donor. Left: CAR-T expansion after each stimulation, Right: Cumulative CAR-T expansion. Arrow indicates the timing of PC3-PSMA challenge. FIG. 2G: Summary of the expansion capacity of AAVS1 and PRDM1 KO CAR T-cells during the restimulation assay with four different donors. FIG. 2H: Gene set enrichment analysis (GSEA) of PRDM1 KO versus AAVS1 KO CAR-T comparing gene signatures related to cell cycle and mitotic DNA replication. CAR-T samples were harvested on day 5 post first tumor challenge. NES: normalized enrichment score, FDR: false discovery rate. FIG. 2I: Early memory marker expression measured by flow cytometry after two consecutive tumor challenges. FIG. 2J: Volcano plot demonstrating the result of differential expression analysis comparing PRDM1 KO CAR-T with control AAVS1 KO CAR-T cells. FIGS. 2K-2M: GSEA of PRDM1 KO versus AAVS1 KO CAR-T comparing gene sets associated with (FIG. 2K) memory T-cells (GSE10239) and (FIG. 2L) metabolism (GO_Fatty acid_Beta oxidation), and (FIG. 2M) KEGG_TCA cycle. All knockout and restimulation experiments were conducted with CAR T-cells manufactured from 4 different healthy donors. RNA-seq experiments were conducted with CAR T-cells manufactured from 2 different healthy donors, each with replicates generated from two independent experiments. *P<0.05, *P<0.01, ***P<0.001, n.s.: not significant (paired t-test).

FIGS. 3A-31: PRDM1 KO increases TCF7 expression and enhances early memory CAR T-cell differentiation in a TCF7-dependent manner. FIG. 3A: TCF7 expression level measured by flow cytometric analysis of PRDM1 KO versus AAVS1 KO CAR T-cells PSMA CAR T-cells derived from n=4 different healthy subjects. FIG. 3B: Analysis of CAR T-cells for transcripts enriched in TCF7⁺ stem cell-like T-cells from LCMV mouse models. FIG. 3C: GSEA of PRDM1 KO versus AAVS1 KO CAR-T comparing gene sets associated with TCF7⁺ memory state (GSE83978) and loss of sternness (GSE84105). FIG. 3D: GSEA of PRDM1 KO versus AAVS1 KO CAR-T comparing gene sets enriched in TCF7⁺ CD8 (left) and TIM3⁺ CD8 clusters (right) observed in mCRPC patient CAR-T infusion products. NES: normalized enrichment score, FDR: false discovery rate. FIG. 3E: Representative histogram of flow cytometric TCF7 expression in PRDM1 and TCF7 KO variants. FIG. 3F: Expansion kinetics during a restimulation assay of gene-edited CAR T-cells. CAR T-cells were challenged with target cell PC3-PSMA every 5 days at a ratio of E:T=3:1. Arrows indicate the timing of PC3-PSMA cell restimulation. Data are mean±S.D. from n=3 independent experiments. FIGS. 3G-3H: CCR7 and CD62L expression following the first in vitro tumor cell challenge. Data represent the mean±S.D. from n=3 independent experiments. FIG. 3I: CAR-T polyfunctionality (IL-2, IFNγ and TNFα) evaluated after 15 hr co-culture with PC3-PSMA cells. Data are mean±S.D. from n=3 independent experiments. *P<0.05, *P<0.01, ***P<0.001, n.s.: not significant (FIGS. 3A, 3F, 3G: two-tailed t-test).

FIGS. 4A-4G: PRDM1 KO marginally enhances solid tumor control despite significant increases in CAR T-cell early memory phenotype and proliferative capacity. CAR T-cells were restimulated five times with PC3-PSMA target cells every 4-5 days at an E:T ratio of 3:1. FIG. 4A: Heat map showing relative effector cytokine secretion levels by AAVS1 KO and PRDM1 KO CAR T-cells after first and fifth tumor cell restimulations. FIG. 4B: Killing kinetics of AAVS1 KO and PRDM1 KO CAR T-cells. CAR T-cells were isolated after the fifth restimulation timepoint and co-cultured with PC3-PSMA cells at a ratio of E:T=3:1. Cytotoxicity was monitored by real-time cellular impedance monitoring technology (xCELLigence). T-cells without CAR transduction were used as a negative control and 20% Tween-20 treatment served as a full lysis control. Data are expressed as mean±S.D. from n=6 individual donors. FIG. 4C: A schematic of high tumor burden PC3-PSMA xenograft mouse model. Male NSG mice were subcutaneously transplanted with 5×10⁶ PC3-PSMA cells, and 3.5×10⁵ PSMA CAR T-cells were given intravenously when tumor volume reached ˜500 mm³. FIG. 4D: Tumor growth monitored by caliper measurements. †=death. FIG. 4E: CAR T-cell expansion kinetics in the peripheral blood. FIG. 4F: Absolute numbers of human T-cells in the peripheral blood on day 38 post-CAR T-cell injection. FIG. 4G: Frequencies of peripheral blood CAR T-cells expressing CCR7 and CD62L, as measured by flow cytometry. Data are shown as mean±S.D.; n=6 for FIGS. 4D and 4G and n=4-5 for FIGS. 4E and 4F. *P<0.05, *P<0.01, ***P<0.001, n.s.: not significant (Mann Whitney U test).

FIGS. 5A-5H: PRDM1 KO CAR T-cells fail to sustain antitumor effector function due to upregulation of exhaustion-related transcription factors (TFs). FIG. 5A: Volcano plot illustrating differential gene expression analysis in PRDM1 KO compared to control AAVS1 KO CAR T-cells after the fourth consecutive tumor cell challenge. FIG. 5B: Heat map showing expression levels of TF genes associated with T-cell exhaustion. RNA-seq experiments were conducted with CAR T-cells manufactured from 2 different subjects, each with replicates generated from two independent experiments. The exhaustion-related TF expression profile reveal in this study appears in the left panel, while a similar profile of exhausted TILs (GSE113221) is shown in the right panel. FIG. 5C: Expansion kinetics of PRDM1 and NR4A3 knockout CAR T-cells during a restimulation assay. PSMA CAR T-cells were challenged with PC3-PSMA tumor cells every 5 days at an E:T ratio of 3:1. Arrows indicate the timing of PC3-PSMA challenge. Data are mean±S.D. from n=3. FIG. 5D-5E: Levels of effector cytokine production by AAVS1 KO, PRDM1 KO, NR4A3 KO and PRDM1/NR4A3 dual KO CAR T-cells. FIG. 5D: Heat map showing effector cytokine secretion level of AAVS1 KO and PRDM1 KO CAR T-cells after first and fifth tumor challenge. FIG. 5E: Graphical summaries of effector cytokine production after the fifth CAR T-cell stimulation with tumor targets. Data are mean±S.D. (n=3). FIG. 5F: Killing kinetics and cytolytic capacity at 36-hours post-CAR T-cell/tumor cell co-culture. FIG. 5G: CAR T-cells were isolated after the fifth round of antigen stimulation and co-cultured with PC3-PSMA tumor cells at an E:T of 3:1 for a ‘stressed’ cytotoxicity assay. Data indicate mean S.D. (n=6). FIGS. 5C-5G: Data are representative of 3 independent experiments performed with engineered CAR T-cells manufactured from 3 different healthy subjects. FIG. 5H: CAR T-cells were repetitively challenged with PC3 or PC3-PSMA tumor targets every 5 days at an E:T ratio of 3:1. The expansion capacity and viability of CAR T-cells were assessed over time. Data are depicted as the mean±S.D. from n=3 independent subjects. Data are representative of 2 independent experiments. *P<0.05, *P<0.01, ***P<0.001, n.s.: not significant (two-tailed unpaired t-test).

FIGS. 6A-6J: Upregulation of exhaustion transcription factors in PRDM1 KO CAR T-cells is attributed to increase in chromatin accessibility and calcineurin-NFAT signaling. FIGS. 6A-6D: Assay for Transposase-Accessible Chromatin using sequencing (ATAC-seq) analysis of AAVS1 KO and PRDM1 KO CAR T-cells. At the end of CAR-T manufacturing, CAR-positive cells were enriched and used for ATAC-seq analysis. FIG. 6A: Volcano plot of differentially accessible regions identified by ATAC-seq analysis. FIG. 6B: Top transcription factor motifs enriched in PRDM1 KO CAR T-cells compared to AAVS1 KO CAR T-cells. FIG. 6C: PRDM1 binding motif enriched in open chromatin regions observed in PRDM1 KO CAR T-cells. FIG. 6D: ATAC-seq tracks of TOX, TOX2, NR4A3 loci. Opened chromatin regions in PRDM1 KO and binding motifs of PRDM1 and NFAT2 are labeled. ATAC-seq experiments were conducted with CAR T-cells manufactured from 2 different subjects, each with replicates generated from two independent experiments. FIG. 6E: Expression of Granzyme B and Perform measured by flow cytometry. FIG. 6F-6G: Cytotoxicity assay to determine the time to kill 50% of target cells (KT₅₀). FIG. 6F: Representative killing kinetics of AAVS1 and PRDM1 KO CAR T-cells. Data are mean±S.D. from n=3. FIG. 6G: Comparison of KT₅₀ between AAVS1 and PRDM1 KO CAR T-cells. Data were generated from 6 independent experiments with CAR T-cells manufactured from 4 different subjects. FIGS. 6H-6I: Expression level of exhaustion-related transcription factors upon repetitive tumor challenges. AAVS1 KO CAR-T and PRDM1 KO CAR T-cells were challenged with PC3-PSMA every 2-4 days at a ratio of E:T=1:1 in presence or absence of 100 nM FK506. Following two consecutive stimulations, CAR T-cells were isolated and expression levels of exhaustion transcription factors were measured. FIG. 6H: TOX expression measured by flow cytometric analysis. FIG. 6I: NR4A2 expression measured by quantitative reverse transcription PCR (qRT-PCR). FIG. 6J: NR4A3 expression measured by qRT-PCR. Data are mean±S.D. from n=3. *P<0.05, *P<0.01, ***P<0.001, n.s.: not significant (two-tailed unpaired t-test).

FIGS. 7A-7M: PRDM1 NR4A3 dual KO enhances in vivo CAR T-cell antitumor activity by preserving TCF1⁺ CD8⁺ T-cells and increasing effector function. FIGS. 7A-7B: Male NSG mice were subcutaneously engrafted with 5×10⁶ PC3-PSMA tumor cells, and 3.5×10⁵ PSMA CAR T-cells were given intravenously when tumor volume reached ˜500 mm³ (n=6-7). FIG. 7A: Kaplan-Meier curves showing overall survival of each group. Gehan-Breslow-Wilcoxon test was used for statistical analysis. FIG. 7B: Tumor growth monitored over time. FIG. 7C: Male NSG mice were intrafemorally injected with 2×10⁵ PC3-PSMA tumor cells. On day 27, 1-2×10⁵ PSMA CAR T-cells were injected intravenously, and tumor burden was measured by bioluminescent imaging. (BLI, (p/sec/cm2/sr)). FIGS. 7D-7H: Tumor and peripheral blood samples were harvested from mice injected subcutaneously with PC3-PSMA tumor cells on day 45 post-tumor implantation when the tumor size was comparable between the groups. Samples were stained with hCD45, murine CD45 (mCD45), CD4, and CD8 antibodies and analyzed with flow cytometry. (FIGS. 7D-7E) PD1 and TIM3, (FIGS. 7F-7G) TIM3 and TCF1 expression in CD45⁺ CD8⁺ T-cells derived from the peripheral blood and tumors. FIG. 7H: Effector cytokine expression levels measured by flow cytometry following ex vivo stimulation of CAR TILs. TILs were activated with 50 ng/mL phorbol 12-myristate 13-acetate (PMA) and 1 ug/mL ionomycin in presence of 5 ug/mL Brefeldin A for 6-hours, followed by staining IFNγ and TNFα staining (n=4-7). A representative flow cytometry plot showing IFNγ and TNFα expression is shown. Statistical analysis in FIGS. 7C-7H was conducted using a Mann Whitney U test; mean±S.E.M. shown. FIG. 7I: A schematic of the NALM-6 xenograft model. Briefly, NSG mice were intravenously injected with 1×10⁶ NALM6-CBG cells. On day 7 post tumor injection, 3×10⁵ gene-edited CD19 or control CAR T-cells were treated (n=7-8). (FIG. 7J) Survival and (FIG. 7K) graphical summaries of longitudinal bioluminescent tumor burden in NSG mice injected with 1×10⁶ NALM-6 CBG cells, followed by treatment with 3×10⁵ gene-edited CD19 or control CAR T-cells (n=7-8). Data are representative of two independent experiments. The Gehan-Breslow-Wilcoxon test was used for survival analysis shown in FIG. 7J. FIG. 7L: A schematic of the NALM-6 rechallenge model is shown. Briefly, NSG mice were intravenously injected with 1×10⁵ NALM6-CBG cells. On day 6 post-tumor injection, 2×10⁶ CD19 CAR T-cells were infused (n=9-10). Surviving mice were rechallenged with a second dose of NALM-6 cells on day 40. FIG. 7M: Longitudinal tumor burden is shown. *P<0.05, *P<0.01, ***P<0.001, n.s.: not significant.

FIGS. 8A-8H: Single-cell RNA-seq study design and subsequent analysis. FIG. 8A: A schematic of study design and sample processing for scRNA-seq analysis of PSMA CAR T-cell infusion products. FIG. 8B: Infusion product analyzed by scRNA-seq using integrated data from five patients (total 20,702 cells that passed QC). Clusters are labeled with cell types (left) and patient origin (right). FIG. 8C: UMAP plots visualizing mRNA transcripts for selected genes, MS4A1, CD3D, CD4, and CD8A, in infusion products. FIG. 8D: Expression of cluster-defining markers. FIG. 8E: Violin plots showing expression level of early memory, cytotoxic, and exhaustion markers in CD8 T-cells. FIG. 8F: Scores of gene signatures associated with T-cell exhaustion (PMID24530057, PMID26123020). *P<0.05, *P<0.01, ***P<0.001, n.s.: not significant (Kruskal-Wallis test with a post hoc Dunn's multiple comparison test). FIG. 8G: Frequency of CCR7⁺CD8⁺, TCF7⁺CD8⁺, TIM3⁺CD8⁺, GZMA⁺CD8⁺ clusters in each infusion product. FIG. 8H: Bubble plot showing the associations between mCRPC patient CAR T-cell infusion product TCF7⁺CD8⁺ and TIM3⁺CD8⁺ gene signature scores, PSA response and peripheral blood (PB) CAR T-cell expansion. CAR T-cell doses and lymphodepletion conditions for each patient are indicated.

FIGS. 9A-9D: Single-cell RNA-seq analysis of infusion product CD4⁺ T-cells. FIG. 9A: Frequencies of CD4⁺ and CD8⁺ T-cells in mCRPC patient CAR T-cell infusion products. FIG. 9B: UMAP plot displaying sub-clustering of infusion product CD4⁺ T-cells. FIGS. 9C-9D: Cluster-defining marker gene expression profiles of CD4⁺ subclusters.

FIG. 10: PSMA CAR expression at the end of CAR-T manufacturing. Flow cytometric histogram depicting PSMA CAR expression levels in PRDM1 KO compared to AAVS1 KO CAR T-cells (representative CAR T-cell data from n=3 different subjects).

FIGS. 11A-11G: in vivo study using CRPC xenograft mouse model to examine the in vivo activity of PRDM1 KO CAR T-cells. FIG. 11A: A schematic of low tumor burden PC3-PSMA xenograft mouse model. FIG. 11B: PC3 growth monitored by bioluminescent imaging. Data are geometric mean±geometric S.D.; n=6, representative of two independent experiments. FIG. 11C: A schematic of NALM6 xenograft model. Briefly, NSG mice were intravenously injected with 1×106 NALM6-CBG cells, and 1×105 CD19 CAR T-cells and negative control, PSMA CAR T-cells, were treated 7 days post CAR-T injection (n=6). FIG. 11D: NALM6 growth monitored by bioluminescent imaging. FIG. 11E: Tumor burden on D16 post CAR-T injection. Data are geometric mean±geometric S.D.; n=6 (Mann Whitney U test). FIG. 11F: Expression of inhibitory receptors (PD1 and LAG3) in human T-cells detected in peripheral blood. Data are mean±S.D.; n=6. (two-tailed t-test). FIG. 11G: Representative flow cytometry plots illustrating gating strategy to characterize the immunophenotype of CAR T-cells in peripheral blood. *P<0.05, *P<0.01, ***P<0.001, n.s.: not significant.

FIGS. 12A-12J: Early memory differentiation phenotypes and cytotoxic profiles of PRDM1/NR4A3 dual KO CAR T-cells. FIG. 12A: Flow cytometric contour plots showing frequencies of gene-edited CAR T-cells expressing TIM-3 and LAG-3 inhibitory receptors. FIG. 12B: Comparison of NR4A3 expression levels in CD19 CAR T-cell infusion products from CLL patients (CR: complete response; PR_{TD}: very good partial response; PR: partial response; no response) (FPKM: Fragments per kilo base of transcript per million mapped fragments). FIG. 12C: Representative Western blots showing BLIMP1 and NR4A3 expression in gene-edited CAR T-cells. FIG. 12D: Granzyme B and Perform expression in CD8⁺ CAR T-cells after five rounds of restimulation with PC3-PSMA tumor targets. FIG. 12E: Expression of early memory T-cell markers (CCR7 and CD62L) on gene-edited CD8⁺ CAR T-cells at 5 days post-tumor challenge. FIG. 12F: Flow cytometric TCF1 expression at 5 days post-tumor challenge. FIG. 12G: Expression of CCR7 and TCF7 in CD4⁺ CAR T-cells at 5 days post-tumor challenge. FIG. 12H: Granzyme B and Perforin expressions in CD4⁺ CAR T-cells after fifth tumor challenge. FIG. 12I: PSMA expression levels of PC3 cell lines. FIG. 12J: CAR T-cells were repetitively challenged with PC3-PSMA^{high} cell lines. After the fifth tumor challenge, PSMA CAR T-cells were isolated and co-cultured with PC3-PSMA^{high} or PC3-PSMA^{low} cell lines. Effector cytokines were measured 24-hours following co-culture. Data depict mean±S.D. (n=3). *P<0.05, *P<0.01, ***P<0.001, n.s.: not significant (two-tailed t-test).

FIGS. 13A-13B: PRDM1 KO increases chromatin accessibility of memory-related genes and NFAT2 expression. FIG. 13A: Gene loci where increased chromatin accessibility in PRDM1 KO CAR T-cells correlate with increased gene expression measured by RNA-seq. Opened chromatin regions in PRDM1 KO and binding motifs of PRDM1 and NFAT2 are labeled. ATAC-seq experiments were conducted with CAR T-cells manufactured from 2 different subjects, each with replications. FIG. 13B: NFAT2 expression measured by flow cytometric analysis.

FIGS. 14A-14L: PRDM1 NR4A3 double KO enhances CAR T-cell anti-tumor efficacy in xenograft mouse models of adoptive cell immunotherapy. FIGS. 14A-14E: AAVS1 KO, PRDM1 KO, NR4A3 KO, and PRDM1/NR4A3 dual KO PSMA CAR T-cells were isolated from subcutaneous PC3-PSMA tumors and immunophenotyped by flow cytometry. FIG. 14A: Absolute numbers of human CD45⁺ (hCD45) T-cells in tumors (left) and the peripheral blood (PB; right) are shown. Frequencies of gene-edited CAR T-cells isolated from the peripheral blood or tumors expressing (FIG. 14B) CD62L and (FIG. 14C) PD1 as well as LAG3. FIG. 14D: Evaluation of the proportions of CAR T-cells expressing TIM3 and TCF1 in the peripheral blood of tumor-bearing mice. FIG. 14E: CAR TILs were reactivated with PMA and ionomycin for 6-hours, followed by intracellular staining for IFNγ, TNFα, and IL-2. FIGS. 14F-14G: NSG mice were subcutaneously injected with the AsPC1 pancreatic cancer cell line. On day 30, when tumor volume reached 300-400 mm³, mesothelin-directed CAR T-cells were intravenously administered, and tumor growth was monitored. FIG. 14H: NSG mice were intravenously injected with 1×10⁶ NALM-6-CBG cells, followed by infusion of 3×10⁵ CD19 CAR T-cells (pBBz, negative control), AAVS1 KO PSMA CAR T-cells, PRDM1 KO PSMA CAR T-cells, NR4A3 KO CAR T-cells or PRDM1 NR4A3 dual KO CAR T-cells at 7-days post-tumor injection (n=7-8). Representative bioluminescent images are shown. FIGS. 14I-14L: Immunophenotyping of CD19 CAR T-cells isolated from NALM-6 engrafted mice at day 24 post-tumor injection. FIG. 14I: The CAR T-cell count in the peripheral blood is shown. (FIG. 14J) Frequencies of CD62L-positive CAR T-cells, (FIG. 14K) PD1 and LAG3 double-positive CAR T-cells and (FIG. 14L) PD1 and TIM3 double-positive T-cells.

FIG. 15: Graphical abstract of how PRDM1/NR4A3 double KO enhances anti-tumor activity of CAR T-cells.

FIG. 16: PRDM1 Knock-out in primary human T cells using CRISPR/Cas9.

FIG. 17: PRDM1 Knock-out increases the proliferative capacity of CAR T cells.

FIG. 18: PRDM1 Knock-out increases the frequency of early memory CAR T cells and prevents progressive differentiation. ND=normal donor; stim.=stimulation; TSCM=stem cell memory T cells; TEM=effector memory T cells; TEMRA=terminally differentiated T cells.

FIG. 19: PRDM1 Knock-out decreases frequencies of CAR T cells expressing inhibitory receptors. ND=normal donor.

FIG. 20: PRDM1 Knock-out enhances/maintains effector cytokine expression by CAR T cells. ND=normal donor; stim.=round of stimulation.

FIG. 21: RNA-seq analysis of CAR T cells reveals that PRDM1 knock-out leads to downregulation of exhaustion and senescence genes and upregulation of genes that maintain early T cell differentiation.

FIGS. 22A-22C: PRDM1 Knock-out enhances the in vivo anti-tumor activity of CAR T cells in association with increased proliferative capacity and early memory differentiation. FIG. 22A: Absolute counts of human T cells in the peripheral blood of mice injected with PRDM1 or AAVS (control) knock-out PSMA CAR T cells or PBS alone. FIG. 22B: Chemiluminescent tumor burden in mice injected with PRDM1 or AAVS (control) knock-out PSMA CAR T cells. BLI=Bioluminescence imaging. FIG. 22C: Differentiation phenotype of peripheral blood PRDM1 or AAVS knock-out CAR T cells at day 48 post-infusion.

FIG. 23: Knock-out (KO) of endogenous TGFβRII in CAR T cells.

FIG. 24: Knock-out (KO) of endogenous TGFβRII in CAR T cells.

FIG. 25: TGFβRII KO PSMA CAR T cells are cytotoxic.

FIG. 26: PRDM1 (P)+TGFβRII (T) Knock-out synergize to enhance the proliferative capacity of CAR T cells.

FIG. 27: PRDM1 (P)+TGFβRII (T) Knock-out synergize to enhance cytokine production by CAR T cells.

FIG. 28: PRDM1 (P)+TGFβRII (T) Knock-out synergize to enhance in vivo CAR T cell anti-tumor efficacy.

### DETAILED DESCRIPTION

The present disclosure provides compositions and methods for modified immune cells or precursors thereof (e.g., modified T cells) comprising a modification in an endogenous gene locus encoding PRDM1 and an exogenous chimeric antigen receptor (CAR) and/or T cell receptor (TCR). Also provided are compositions and methods for modified immune cells or precursors thereof (e.g., modified T cells) comprising a modification in an endogenous gene locus encoding PRDM1, a modification in an endogenous gene locus encoding NR4A3, and an exogenous CAR and/or TCR. Also provided are compositions and methods for modified immune cells or precursors thereof (e.g., modified T cells) comprising a modification in an endogenous gene locus encoding PRDM1, a modification in an endogenous gene locus encoding TGFβRII, and an exogenous CAR and/or TCR. In some embodiments, the modified immune cells are genetically edited such that the expression of PRDM1, NR4A3, and/or TGFβRII is downregulated or knocked out. These genetically edited modified immune cells have enhanced immune function. In some embodiments, the genetically edited modified immune cells provided herein are resistant to T cell exhaustion.

Herein, TGFβRII armored PSMA CAR T-cell infusion products of metastatic castration-resistant prostate cancer (mCRPC) patients (clinicaltrials.gov NCT03089203) were analyzed to identify cellular populations and molecular features that are associated with clinical response. Using scRNA-seq analysis, TCF7⁺ CD8 T-cells were identified in PSMA CAR-T infusion products that are associated with stem cell-like T-cells and effective clinical response and TIM3⁺ CD8 T-cells that are associated with exhaustion, poor persistence, low peak expansion, and poor clinical response. It was hypothesized that deletion of PR/SET domain 1 (PRDM1) which is upregulated in TIM3⁺ CD8 T-cells and downregulated in TCF7⁺ CD8 T-cells can enrich desirable, and deplete undesirable, populations during manufacturing. Consistent with this hypothesis, PRDM1 knockout (KO) increased TCF7⁺ early memory T-cells and decreased TIM3⁺ CD8 signature, which significantly improved CAR T-cell persistence and expansion. Moreover, a novel mechanism by which PRDM1 regulates exhaustion TFs was identified. Deletion of NR4A3 together with PRDM1 further rectified CAR-T dysfunction and induced durable effector function during chronic CAR activation, which led to significant improvement of in vivo antitumor activity.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the compositions provided herein. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present disclosure can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Insertion/deletion”, commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrastemal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Modified Immune Cells

The present disclosure provides modified immune cells or precursors thereof (e.g., T cells) comprising a modification in an endogenous gene locus encoding PRDM1, NR4A3, and/or TGFβRII; and an exogenous T cell receptor (TCR) and/or a chimeric antigen receptor (CAR). The cells can include any CAR or TCR known in the art or described herein.

In one embodiment, the disclosure provides modified immune cells or precursors thereof (e.g., T cells), comprising: a) a modification in an endogenous gene locus encoding PRDM1, wherein the modification is capable of downregulating gene expression of, or knocking out, endogenous PRDM1, and b) an exogenous T cell receptor (TCR) and/or chimeric antigen receptor (CAR) comprising affinity for an antigen on a target cell.

Also provided are modified immune cells or precursor thereofs (e.g., T cells) comprising: a) a modification in an endogenous gene locus encoding PRDM1, wherein the modification is capable of downregulating gene expression of, or knocking out, endogenous PRDM1, b) a modification in an endogenous gene locus encoding NR4A3, wherein the modification is capable of downregulating gene expression of, or knocking out, endogenous NR4A3, c) and an exogenous T cell receptor (TCR) and/or chimeric antigen receptor (CAR) comprising affinity for an antigen on a target cell.

Also provided are modified immune cells or precursors thereof (e.g., T cells), comprising: a) a modification in an endogenous gene locus encoding PRDM1, wherein the modification is capable of downregulating gene expression of, or knocking out, endogenous PRDM1, b) a modification in an endogenous gene locus encoding TGFβRII, wherein the modification is capable of downregulating gene expression of, or knocking out, endogenous TGFβRII, and c) an exogenous T cell receptor (TCR) and/or chimeric antigen receptor (CAR) comprising affinity for an antigen on a target cell.

The present disclosure provides gene edited modified cells. In some embodiments, a modified cell (e.g., a modified cell comprising an exogenous CAR and/or TCR) of the present disclosure is genetically edited to disrupt the expression of an endogenous gene locus encoding PRDM1, NR4A3, and/or TGFβRII. In some embodiments, the gene-edited immune cells (e.g., T cells) have a downregulation, reduction, deletion, elimination, knockout or disruption in expression of the endogeneous PRDM1, NR4A3, and/or TGFβRII.

Various gene editing technologies are known to those skilled in the art. Gene editing technologies include, without limitation, homing endonucleases, zinc-finger nucleases (ZFNs), transcription activator-like effector (TALE) nucleases (TALENs), and clustered regularly interspaced short palindromic repeats (CRISPR)-associated protein 9 (Cas9). Homing endonucleases generally cleave their DNA substrates as dimers, and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. The Cas9 nuclease is targeted to DNA sequences complementary to the targeting sequence within the single guide RNA (gRNA) located immediately upstream of a compatible protospacer adjacent motif (PAM). Accordingly, one of skill in the art would be able to select the appropriate gene editing technology for the present disclosure.

In some aspects, the modification is carried out by gene editing using an RNA-guided nuclease such as a CRISPR-Cas system, such as CRISPR-Cas9 system, specific for the gene (e.g., PRDM1, NR4A3, and/or TGFβRII) being disrupted. In some embodiments, an agent containing a Cas9 and a guide RNA (gRNA) containing a targeting domain, which targets a region of the genetic locus, is introduced into the cell. In some embodiments, the agent is or comprises a ribonucleoprotein (RNP) complex of a Cas9 polypeptide and a gRNA (Cas9/gRNA RNP). In some embodiments, the introduction includes contacting the agent or portion thereof with the cells in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap.

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and TCR T cells. The CRISPR/Cas9 system can simultaneously target multiple genomic loci by co-expressing a single Cas9 protein with two or more gRNAs, making this system suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The REC I domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, a pAd5F35-CRISPR vector. In other embodiments, the Cas expression vector induces expression of Cas9 endonuclease. Other endonucleases may also be used, including but not limited to, Cas12a (Cpf1), T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). Other inducible promoters known by those of skill in the art can also be used. The inducing agent can be a selective condition (e.g., exposure to an agent, for example an antibiotic) that results in induction of the inducible promoter. This results in expression of the Cas expression vector.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” or “target sequence” is the DNA sequence in a genome of a cell that is complementary to the guide sequence of the gRNA.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of a CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas nuclease, a crRNA, and a tracrRNA could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In some embodiments, the CRISPR/Cas is derived from a type II CRISPR/Cas system. In other embodiments, the CRISPR/Cas system is derived from a Cas9 nuclease. Exemplary Cas9 nucleases that may be used in the present disclosure include, but are not limited to, S. pyogenes Cas9 (SpCas9), S. aureus Cas9 (SaCas9), S. thermophilus Cas9 (StCas9), N. meningitidis Cas9 (NmCas9), C. jejuni Cas9 (CjCas9), and Geobacillus Cas9 (GeoCas9).

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present disclosure. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present disclosure may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, guide RNA(s) and Cas9 can be delivered to a cell as a ribonucleoprotein (RNP) complex (e.g., a Cas9/RNA-protein complex). RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI). In some embodiments, the Cas9/RNA-protein complex is delivered into a cell by electroporation.

In some embodiments, a gene edited modified cell of the present disclosure is edited using CRISPR/Cas9 to disrupt an endogenous gene locus encoding PRDM1, NR4A3, and/or TGFβRII. Suitable gRNAs for use in disrupting PRDM1, NR4A3, and/or TGFβRII are set forth in SEQ ID NO: 2 and SEQ ID NO: 4. It will be understood to those of skill in the art that guide RNA sequences may be recited with a thymidine (T) or a uridine (U) nucleotide.

Accordingly, provided in the disclosure is a modified immune cell or precursor cell thereof comprising a CRISPR-mediated modification in an endogenous gene locus encoding PRDM1, wherein the modification is capable of downregulating gene expression of, or knocking out, endogenous PRDM1; and an exogenous CAR and/or TCR comprising affinity for an antigen on a target cell. Also provided in the disclosure is a modified immune cell or precursor cell thereof comprising a CRISPR-mediated modification in an endogenous gene locus encoding PRDM1, wherein the modification is capable of downregulating gene expression of, or knocking out, endogenous PRDM1, a CRISPR-mediated modification in an endogenous gene locus encoding NR4A3, wherein the modification is capable of downregulating gene expression of, or knocking out, endogenous NR4A3; and an exogenous CAR and/or TCR comprising affinity for an antigen on a target cell, Also provided in the disclosure is a modified immune cell or precursor cell thereof comprising a CRISPR-mediated modification in an endogenous gene locus encoding PRDM1, wherein the modification is capable of downregulating gene expression of, or knocking out, endogenous PRDM1, a CRISPR-mediated modification in an endogenous gene locus encoding TGFβRII, wherein the modification is capable of downregulating gene expression of, or knocking out, endogenous TGFβRII; and an exogenous CAR and/or TCR comprising affinity for an antigen on a target cell.

Non-limiting types of CRISPR-mediated modifications include a substitution, an insertion, a deletion, and an insertion/deletion (INDEL). The modification can be located in any part of the endogenous gene locus encoding PRDM1, NR4A3, and/or TGFβRII, including but not limited to an exon, a splice donor, or a splice acceptor.

In certain embodiments, the modified cell is resistant to cell exhaustion. In certain embodiments, the modified cell is an autologous cell. In certain embodiments, the modified cell is a cell isolated from a human subject. In certain embodiments, the modified cell is a modified immune cell. In certain embodiments, the modified cell is a modified T cell. In certain embodiments, the modified cell is a modified T cell resistant to T cell exhaustion.

In some aspects, the provided compositions and methods include those in which at least or greater than about 50%, 60%, 65%, 70%, 75%, 80%, 85%, 90% or 95% of immune cells in a composition of immune cells contain the desired genetic modification. For example, about 50%, 60%, 65%, 70%, 75%, 80%, 85%, 90% or 95% of immune cells in a composition of cells into which an agent (e.g. gRNA/Cas9) for knockout or genetic disruption of endogenous gene (e.g., PRDM1, NR4A3, and/or TGFβRII) was introduced contain the genetic disruption; do not express the targeted endogenous polypeptide, do not contain a contiguous and/or functional copy of the targeted gene. In some embodiments, the methods, compositions and cells according to the present disclosure include those in which at least or greater than about 50%, 60%, 65%, 70%. 75%, 80%, 85%, 90% or 95% of cells in a composition of cells into which an agent (e.g. gRNA/Cas9) for knockout or genetic disruption of a targeted gene was introduced do not express the targeted polypeptide, such as on the surface of the immune cells. In some embodiments, at least or greater than about 50%, 60%, 65%, 70%, 75%, 80%, 85%, 90% or 95% of cells in a composition of cells into which an agent (e.g. gRNA/Cas9) for knockout or genetic disruption of the targeted gene was introduced are knocked out in both alleles, i.e. comprise a biallelic deletion, in such percentage of cells.

In some embodiments, provided are compositions and methods in which the Cas9-mediated cleavage efficiency (% indel) in or near the targeted gene (e.g. within or about within 100 base pairs, within or about within 50 base pairs, or within or about within 25 base pairs or within or about within 10 base pairs upstream or downstream of the cut site) is at least or greater than about 50%, 60%, 65%, 70%, 75%, 80%, 85%, 90% or 95% in cells of a composition of cells into which an agent (e.g. gRNA/Cas9) for knockout or genetic disruption of a targeted gene has been introduced.

In some embodiments, the provided cells, compositions and methods results in a reduction or disruption of signals delivered via the endogenous in at least or greater than about 50%, 60%, 65%, 70%, 75%, 80%, 85%, 90% or 95% of cells in a composition of cells into which an agent (e.g. gRNA/Cas9) for knockout or genetic disruption of a targeted gene was introduced.

In some embodiments, compositions according to the provided disclosure that comprise cells engineered with a recombinant receptor and comprise the reduction, deletion, elimination, knockout or disruption in expression of an endogenous gene (e.g. genetic disruption of PRDM1, NR4A3, and/or TGFβRII) retain the functional property or activities of the receptor compared to the receptor expressed in engineered cells of a corresponding or reference composition comprising the receptor but do not comprise the genetic disruption of a gene or express the polypeptide when assessed under the same conditions. In some embodiments, the engineered cells of the provided compositions retain a functional property or activity compared to a corresponding or reference composition comprising engineered cells in which such are engineered with the recombinant receptor but do not comprise the genetic disruption or express the targeted polypeptide when assessed under the same conditions. In some embodiments, the cells retain cytotoxicity, proliferation, survival or cytokine secretion compared to such a corresponding or reference composition.

In some embodiments, the immune cells in the composition retain a phenotype of the immune cell or cells compared to the phenotype of cells in a corresponding or reference composition when assessed under the same conditions. In some embodiments, cells in the composition include naïve cells, effector memory cells, central memory cells, stem central memory cells, effector memory cells, and long-lived effector memory cells. In some embodiments, the percentage of T cells, or T cells expressing the CAR and/or TCR, and comprising the genetic disruption of a targeted gene (e.g., PRDM1, NR4A3, and/or TGFβRII) exhibit a non-activated, long-lived memory or central memory phenotype that is the same or substantially the same as a corresponding or reference population or composition of cells engineered with the recombinant receptor but not containing the genetic disruption. In some embodiments, such property, activity or phenotype can be measured in an in vitro assay, such as by incubation of the cells in the presence of an antigen targeted by the CAR and/or TCR, a cell expressing the antigen and/or an antigen-receptor activating substance. In some embodiments, any of the assessed activities, properties or phenotypes can be assessed at various days following electroporation or other introduction of the agent, such as after or up to 3, 4, 5, 6, 7 days. In some embodiments, such activity, property or phenotype is retained by at least 80%, 85%, 90%, 95% or 100% of the cells in the composition compared to the activity of a corresponding composition containing cells engineered with the recombinant receptor but not comprising the genetic disruption of the targeted gene when assessed under the same conditions.

As used herein, reference to a “corresponding composition” or a “corresponding population of immune cells” (also called a “reference composition” or a “reference population of cells”) refers to immune cells (e.g., T cells) obtained, isolated, generated, produced and/or incubated under the same or substantially the same conditions, except that the immune cells or population of immune cells were not introduced with the agent. In some aspects, except for not containing introduction of the agent, such immune cells are treated identically or substantially identically as immune cells that have been introduced with the agent, such that any one or more conditions that can influence the activity or properties of the cell, including the upregulation or expression of the inhibitory molecule, is not varied or not substantially varied between the cells other than the introduction of the agent.

Methods and techniques for assessing the expression and/or levels of T cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, antigen receptor (e.g. CAR)-expressing cells can be detected by flow cytometry or other immunoaffinity based method for expression of a marker unique to such cells, and then such cells can be co-stained for another T cell surface marker or markers.

In some embodiments, the cells, compositions and methods provide for the deletion, knockout, disruption, or reduction in expression of the target gene in immune cells (e.g. T cells) to be adoptively transferred (such as cells engineered to express an exogenous CAR and/or TCR). In some embodiments, the methods are performed ex vivo on primary cells, such as primary immune cells (e.g. T cells) from a subject. In some aspects, methods of producing or generating such genetically engineered T cells include introducing into a population of cells containing immune cells (e.g. T cells) one or more nucleic acid encoding a recombinant receptor (e.g. exogenous CAR and/or TCR) and an agent or agents that is capable of disrupting, a gene that encode the endogenous receptor to be targeted. As used herein, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors.

The population of cells containing T cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4+, CD8+ or CD4+ and CD8+ T cells. In some embodiments, the step of introducing the nucleic acid encoding a genetically engineered antigen receptor and the step of introducing the agent (e.g. Cas9/gRNA RNP) can occur simultaneously or sequentially in any order. In some embodiments, subsequent to introduction of the exogenous receptor and one or more gene editing agents (e.g. Cas9/gRNA RNP), the cells are cultured or incubated under conditions to stimulate expansion and/or proliferation of cells.

Thus, provided are cells, compositions and methods that enhance immune cell, such as T cell, function in adoptive cell therapy, including those offering improved efficacy, such as by increasing activity and potency of administered genetically engineered cells, while maintaining persistence or exposure to the transferred cells over time. In some embodiments, the genetically engineered cells, exhibit increased expansion and/or persistence when administered in vivo to a subject, as compared to certain available methods. In some embodiments, the provided immune cells exhibit increased persistence when administered in vivo to a subject. In some embodiments, the persistence of genetically engineered immune cells, in the subject upon administration is greater as compared to that which would be achieved by alternative methods, such as those involving administration of cells genetically engineered by methods in which T cells were not introduced with an agent that reduces expression of or disrupts a gene encoding an endogenous receptor. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the CAR and/or TCR in the blood or serum or organ or tissue (e.g., disease site) of the subject. In some aspects, persistence is quantified as copies of DNA or plasmid encoding the exogenous receptor per microgram of DNA, or as the number of receptor-expressing cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the CAR and/or TCR can be used to distinguish the administered cells from endogenous cells in a subject.

### C. Chimeric Antigen Receptors

The present disclosure provides compositions and methods for modified immune cells or precursors thereof, e.g., modified T cells, comprising a chimeric antigen receptor (CAR). Thus, in some embodiments, the immune cell has been genetically modified to express the CAR. CARs are well-known in the art (see, e.g., WO2014153270A1, WO2014130657A1, and WO2012079000A1). CARs of the present disclosure comprise an antigen binding domain, a transmembrane domain, and an intracellular domain. In certain embodiments, the modified immune cell or precursor thereof comprises a modification in an endogenous gene locus encoding PRDM1 that is capable of downregulating gene expression of, or knocking out, endogenous PRDM1, and an exogeneous CAR comprising affinity for an antigen on a target cell. In certain embodiments, the modified immune cell or precursor thereof comprises a modification in an endogenous gene locus encoding PRDM1 that is capable of downregulating gene expression of, or knocking out, endogenous PRDM1, a modification in an endogenous gene locus encoding NR4A3 that is capable of downregulating gene expression of, or knocking out, endogenous NR4A3, and an exogeneous CAR comprising affinity for an antigen on a target cell. In certain embodiments, the modified immune cell or precursor thereof comprises a modification in an endogenous gene locus encoding PRDM1 that is capable of downregulating gene expression of, or knocking out, endogenous PRDM1, a modification in an endogenous gene locus encoding TGFβRII that is capable of downregulating gene expression of, or knocking out, endogenous TGFβRII, and an exogeneous CAR comprising affinity for an antigen on a target cell. The invention should be construed to include any CAR disclosed herein or known in the art.

The antigen binding domain may be operably linked to another domain of the CAR, such as the transmembrane domain or the intracellular domain, both described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present disclosure. A subject CAR of the present disclosure may also include a hinge domain as described herein. A subject CAR of the present disclosure may also include a spacer domain as described herein. In some embodiments, each of the antigen binding domain, transmembrane domain, and intracellular domain is separated by a linker.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen on a target cell. The target antigen may include any type of protein, or epitope thereof, associated with the target cell. For example, the CAR may comprise affinity to a target antigen on a target cell that indicates a particular disease state of the target cell.

In some embodiments, the target cell antigen is a tumor associated antigen (TAA). Examples of tumor associated antigens (TAAs), include but are not limited to, differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the 5 human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3CA 27.29BCAA, CA 195, CA 242, CA-50, CAM43, CD68P1, CO-029, FGF-5, G250, Ga733EpCAM, HTgp-175, M344, MA-50, MG7-Ag, MOV18, Folate Receptor alpha, NB/70K, NY-CO-1, RCAS1, SDCCAG16, TA-90Mac-2 binding protein\cyclophilin C-associated protein, TAAL6, TAG72, TLP, and TPS. In a preferred embodiment, the antigen binding domain of the CAR targets an antigen that includes but is not limited to CD19, CD20, CD22, BCMA ROR1, Mesothelin, CD33/IL3Ra, c-Met, PSMA, PSCA, Glycolipid F77, EGFRvIII, GD-2, Tn-Mucd, NY-ESO-1 TCR, MAGE A3 TCR, and the like.

Depending on the desired antigen to be targeted, the CAR can be engineered to include the appropriate antigen binding domain that is specific to the desired antigen target. For example, if CD19 is the desired antigen that is to be targeted, an antibody for CD19 can be used as the antigen bind moiety for incorporation into the CAR.

In one embodiment, the target cell antigen is PSMA. As such, in one embodiment, a CAR of the present disclosure has affinity for PSMA on a target cell. In one embodiment, the target cell antigen is CD19. As such, in one embodiment, a CAR of the present disclosure has affinity for CD19 on a target cell. This should not be construed as limiting in any way, as a CAR having affinity for any target antigen is suitable for use in a composition or method of the present disclosure.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. n some embodiments, the target-specific binding domain is a human target-specific binding domain, e.g., the target-specific binding domain is of human origin. In one embodiment, a CAR of the present disclosure having affinity for CD19 on a target cell may comprise a CD19 binding domain.

In some embodiments, a CAR of the present disclosure may have affinity for one or more target antigens on one or more target cells. In some embodiments, a CAR may have affinity for one or more target antigens on a target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

In some embodiments, the antigen binding domain is selected from the group consisting of an antibody, an antigen binding fragment (Fab), and a single-chain variable fragment (scFv). In some embodiments, a PSMA binding domain of the present disclosure is selected from the group consisting of a PSMA-specific antibody, a PSMA-specific Fab, and a PSMA-specific scFv. In one embodiment, a PSMA binding domain is a PSMA-specific antibody. In one embodiment, a PSMA binding domain is a PSMA-specific Fab. In one embodiment, a PSMA binding domain is a PSCA-specific scFv. In some embodiments, a CD19 binding domain of the present disclosure is selected from the group consisting of a CD19-specific antibody, a CD19-specific Fab, and a CD19-specific scFv. In one embodiment, a CD19 binding domain is a CD19-specific antibody. In one embodiment, a CD19 binding domain is a CD19-specific Fab. In one embodiment, a CD19 binding domain is a CD19-specific scFv.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. In some embodiments, the antigen binding domain portion comprises a mammalian antibody or a fragment thereof he choice of antigen binding domain may depend upon the type and number of antigens that are present on the surface of a target cell.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., PSMA binding domain) comprises an scFv having the configuration from N-terminus to C-terminus, VH—linker—VL. In some embodiments, the antigen binding domain comprises an scFv having the configuration from N-terminus to C-terminus, VL—linker—VH. Those of skill in the art would be able to select the appropriate configuration for use in the present disclosure.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 9), (GGGS)ₙ (SEQ ID NO: 10), and (GGGGS)ₙ (SEQ ID NO: 11), where n represents an integer of at least 1. Exemplary linker sequences can comprise amino acid sequences including, without limitation, GGSG (SEQ ID NO:12), GGSGG (SEQ ID NO:13), GSGSG (SEQ ID NO:14), GSGGG (SEQ ID NO:15), GGGSG (SEQ ID NO:16), GSSSG (SEQ ID NO:17), GGGGS (SEQ ID NO:18), GGGGSGGGGSGGGGS (SEQ ID NO:19) and the like. Those of skill in the art would be able to select the appropriate linker sequence for use in the present disclosure. In one embodiment, an antigen binding domain of the present disclosure comprises a heavy chain variable region (VH) and a light chain variable region (VL), wherein the VH and VL is separated by the linker sequence having the amino acid sequence GGGGSGGGGSGGGGS (SEQ ID NO:19), which may be encoded by the nucleic acid sequence GGTGGCGGTGGCTCGGGCGGTGGTGGGTCGGGTGGCGGCGGATCT (SEQ ID NO: 20).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof.

### Transmembrane Domain

CARs of the present disclosure may comprise a transmembrane domain that connects the antigen binding domain of the CAR to the intracellular domain of the CAR. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain of particular use in this disclosure include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in a subject CAR.

In some embodiments, the transmembrane domain further comprises a hinge region. A subject CAR of the present disclosure may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)ₙ, glycine-serine polymers (including, for example, (GS)ₙ, (GSGGS)ₙ (SEQ ID NO:9) and (GGGS)ₙ (SEQ ID NO:10), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO:12), GGSGG (SEQ ID NO:13), GSGSG (SEQ ID NO:14), GSGGG (SEQ ID NO:15), GGGSG (SEQ ID NO:16), GSSSG (SEQ ID NO:17), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO:21); CPPC (SEQ ID NO:22); CPEPKSCDTPPPCPR (SEQ ID NO:23) (see, e.g., Glaser et al., J. Biol. Chem. (2005) 280:41494-41503); ELKTPLGDTTHT (SEQ ID NO:24); KSCDKTHTCP (SEQ ID NO:25); KCCVDCP (SEQ ID NO:26); KYGPPCP (SEQ ID NO:27); EPKSCDKTHTCPPCP (SEQ ID NO:28) (human IgG1 hinge); ERKCCVECPPCP (SEQ ID NO:29) (human IgG2 hinge); ELKTPLGDTTHTCPRCP (SEQ ID NO:30) (human IgG3 hinge); SPNMVPHAHHAQ (SEQ ID NO:31) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:32); see, e.g., Yan et al., J. Biol. Chem. (2012) 287: 5891-5897. In one embodiment, the hinge region can comprise an amino acid sequence derived from human CD8, or a variant thereof.

### Intracellular Signaling Domain

A subject CAR of the present disclosure also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the CAR include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the (chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDIId, ITGAE, CD103, ITGAL, CDIIa, LFA-1, ITGAM, CDlib, ITGAX, CDIIc, ITGBl, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present disclosure include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceRl gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, A1504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. n one embodiment, an intracellular signaling domain suitable for use in a CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen binding domains described herein, any of the transmembrane domains described herein, or any of the other domains described herein that may be included in the CAR.

### D. T Cell Receptors

The present disclosure provides compositions and methods for modified immune cells or precursors thereof (e.g., modified T cells) comprising an exogenous T cell receptor (TCR). Thus, in some embodiments, the cell has been altered to contain specific T cell receptor (TCR) genes (e.g., a nucleic acid encoding an alpha/beta TCR, or gamma/delta TCR). TCRs or antigen-binding portions thereof include those that recognize a peptide epitope or T cell epitope of a target polypeptide, such as an antigen of a tumor, viral or autoimmune protein. In certain embodiments, the TCR has binding specificity for a tumor associated antigen, e.g., human NY-ESO-1. In certain embodiments, the immune cell or precursor thereof comprises a modification in the endogenous gene locus encoding PRDM1 that is capable of downregulating gene expression of the endogenous PRDM1, and an exogeneous TCR comprising affinity for an antigen on a target cell. In certain embodiments, the immune cell or precursor thereof comprises a modification in the endogenous gene locus encoding PRDM1 that is capable of downregulating gene expression of the endogenous PRDM1, a modification in the endogenous gene locus encoding NR4A3 that is capable of downregulating gene expression of the endogenous NR4A3, and an exogeneous TCR comprising affinity for an antigen on a target cell. In certain embodiments, the immune cell or precursor thereof comprises a modification in the endogenous gene locus encoding PRDM1 that is capable of downregulating gene expression of the endogenous PRDM1, a modification in the endogenous gene locus encoding TGFβRII that is capable of downregulating gene expression of the endogenous TGFβRII, and an exogeneous TCR comprising affinity for an antigen on a target cell.

A TCR is a disulfide-linked heterodimeric protein comprised of six different membrane bound chains that participate in the activation of T cells in response to an antigen. There exists alpha/beta TCRs and gamma/delta TCRs. An alpha/beta TCR comprises a TCR alpha chain and a TCR beta chain. T cells expressing a TCR comprising a TCR alpha chain and a TCR beta chain are commonly referred to as alpha/beta T cells. Gamma/delta TCRs comprise a TCR gamma chain and a TCR delta chain. T cells expressing a TCR comprising a TCR gamma chain and a TCR delta chain are commonly referred to as gamma/delta T cells. In certain embodiments, a TCR of the present disclosure is a TCR comprising a TCR alpha chain and a TCR beta chain. In certain embodiments, a TCR of the present disclosure is a TCR comprising a TCR gamma chain and a TCR delta chain.

The TCR alpha chain and the TCR beta chain are each comprised of two extracellular domains, a variable region and a constant region. The TCR alpha chain variable region and the TCR beta chain variable region are required for the affinity of a TCR to a target antigen. Each variable region comprises three hypervariable or complementarity-determining regions (CDRs) which provide for binding to a target antigen. The constant region of the TCR alpha chain and the constant region of the TCR beta chain are proximal to the cell membrane. A TCR further comprises a transmembrane region and a short cytoplasmic tail. CD3 molecules are assembled together with the TCR heterodimer. CD3 molecules comprise a characteristic sequence motif for tyrosine phosphorylation, known as immunoreceptor tyrosine-based activation motifs (ITAMs). Proximal signaling events are mediated through the CD3 molecules, and accordingly, TCR-CD3 complex interaction plays an important role in mediating cell recognition events.

Stimulation of TCR is triggered by major histocompatibility complex molecules (MHCs) on antigen presenting cells that present antigen peptides to T cells and interact with TCRs to induce a series of intracellular signaling cascades. Engagement of the TCR initiates both positive and negative signaling cascades that result in cellular proliferation, cytokine production, and/or activation-induced cell death.

A TCR of the present disclosure can be a wild-type TCR, a high affinity TCR, and/or a chimeric TCR. A high affinity TCR may be the result of modifications to a wild-type TCR that confers a higher affinity for a target antigen compared to the wild-type TCR. A high affinity TCR may be an affinity-matured TCR. Methods for modifying TCRs and/or the affinity-maturation of TCRs are known to those of skill in the art. Techniques for engineering and expressing TCRs include, but are not limited to, the production of TCR heterodimers which include the native disulphide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840).

In some embodiments, the exogenous TCR is a full TCR or an antigen-binding portion or antigen-binding fragment thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable 3 chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

In some embodiments, the variable domains of the TCR contain hypervariable loops, or CDRs, which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al, Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR contains a variable alpha domain (V_{α}) and/or a variable beta domain (V_{β}) or antigen-binding fragments thereof. In some embodiments, the α-chain and/or β-chain of a TCR also can contain a constant domain, a transmembrane domain and/or a short cytoplasmic tail (see, e.g., Janeway et al., Immunobiology: The Immune System in Health and Disease, 3 Ed., Current Biology Publications, p. 4:33, 1997). In some embodiments, the α chain constant domain is encoded by the TRAC gene (IMGT nomenclature) or is a variant thereof. In some embodiments, the β chain constant region is encoded by TRBC1 or TRBC2 genes (IMGT nomenclature) or is a variant thereof. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

It is within the level of a skilled artisan to determine or identify the various domains or regions of a TCR. In some aspects, residues of a TCR are known or can be identified according to the International Immunogenetics Information System (IMGT) numbering system (see e.g. www.imgt.org; see also, Lefranc et al. (2003) Developmental and Comparative Immunology, 2&; 55-77; and The T Cell Factsbook 2nd Edition, Lefranc and LeFranc Academic Press 2001). Using this system, the CDR1 sequences within a TCR Va chain and/or Vβ chain correspond to the amino acids present between residue numbers 27-38, inclusive, the CDR2 sequences within a TCR Va chain and/or Vβ chain correspond to the amino acids present between residue numbers 56-65, inclusive, and the CDR3 sequences within a TCR Va chain and/or Vβ chain correspond to the amino acids present between residue numbers 105-117, inclusive. The IMGT numbering system should not be construed as limiting in any way, as there are other numbering systems known to those of skill in the art, and it is within the level of the skilled artisan to use any of the numbering systems available to identify the various domains or regions of a TCR.

In some embodiments, the TCR may be a heterodimer of two chains α and β (or γ and δ) that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains. In some embodiments, each of the constant and variable domains contain disulfide bonds formed by cysteine residues.

In some embodiments, the TCR for engineering cells as described is one generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, the T-cells can be a cultured T cell hybridoma or clone. In some embodiments, the TCR or antigen-binding portion thereof can be synthetically generated from knowledge of the sequence of the TCR. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15: 169-180 and Cohen et al. (2005) J Immunol. 175:5799-5808. In some embodiments, phage display is used to isolate TCRs against a target antigen (see, e.g., Varela-Rohena et al. (2008) Nat Med. 14: 1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments as described, the TCR can contain an introduced disulfide bond or bonds. In some embodiments, the native disulfide bonds are not present. In some embodiments, the one or more of the native cysteines (e.g. in the constant domain of the α chain and 3 chain) that form a native interchain disulfide bond are substituted with another residue, such as with a serine or alanine. In some embodiments, an introduced disulfide bond can be formed by mutating non-cysteine residues on the alpha and beta chains, such as in the constant domain of the α chain and 3 chain, to cysteine. Exemplary non-native disulfide bonds of a TCR are described in published International PCT No. WO2006/000830 and WO2006/037960. In some embodiments, cysteines can be introduced at residue Thr48 of the α chain and Ser57 of the 3 chain, at residue Thr45 of the α chain and Ser77 of the 3 chain, at residue Tyr10 of the α chain and Ser17 of the 3 chain, at residue Thr45 of the α chain and Asp59 of the 3 chain and/or at residue Ser15 of the α chain and Glu15 of the β chain. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

In some embodiments, the TCR chains contain a transmembrane domain. In some embodiments, the transmembrane domain is positively charged. In some cases, the TCR chain contains a cytoplasmic tail. In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3y, CD35, CD3s and CD3ζ (chains) contain one or more immunoreceptor tyrosine-based activation motif or IT AM that are involved in the signaling capacity of the TCR complex.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). A TCR may be cell-bound or in soluble form. In some embodiments, for purposes of the provided methods, the TCR is in cell-bound form expressed on the surface of a cell. In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR 3 chain variable region sequence is fused to the N terminus a sequence corresponding to a TCR 3 chain constant region extracellular sequence, the first and second polypeptides being linked by a disulfide bond. In some embodiments, the bond can correspond to the native interchain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane. In some embodiments, a dTCR contains a TCR α chain containing a variable a domain, a constant α domain and a first dimerization motif attached to the C-terminus of the constant α domain, and a TCR 3 chain comprising a variable 3 domain, a constant 3 domain and a first dimerization motif attached to the C-terminus of the constant 3 domain, wherein the first and second dimerization motifs easily interact to form a covalent bond between an amino acid in the first dimerization motif and an amino acid in the second dimerization motif linking the TCR α chain and TCR 3 chain together.

In some embodiments, the TCR is a scTCR, which is a single amino acid strand containing an α chain and a β chain that is able to bind to MHC-peptide complexes. Typically, a scTCR can be generated using methods known to those of skill in the art, See e.g., International published PCT Nos. WO 96/13593, WO 96/18105, WO99/18129, WO04/033685, WO2006/037960, WO2011/044186; U.S. Pat. No. 7,569,664; and Schlueter, C. J. et al. J. Mol. Biol. 256, 859 (1996). In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment. In some embodiments, a scTCR contains a first segment constituted by an amino acid sequence corresponding to a TCR β chain variable region, a second segment constituted by an amino acid sequence corresponding to a TCR α chain variable region sequence fused to the N terminus of an amino acid sequence corresponding to a TCR α chain constant domain extracellular sequence, and a linker sequence linking the C terminus of the first segment to the N terminus of the second segment. In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment. In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence comprising an α chain extracellular constant domain sequence and transmembrane sequence, and, optionally, a linker sequence linking the C terminus of the first segment to the N terminus of the second segment. In some embodiments, for the scTCR to bind an MHC-peptide complex, the α and β chains must be paired so that the variable region sequences thereof are orientated for such binding. Various methods of promoting pairing of an α and β in a scTCR are well known in the art. In some embodiments, a linker sequence is included that links the α and β chains to form the single polypeptide strand. In some embodiments, the linker should have sufficient length to span the distance between the C terminus of the α chain and the N terminus of the β chain, or vice versa, while also ensuring that the linker length is not so long so that it blocks or reduces bonding of the scTCR to the target peptide-MHC complex. In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P-, wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, a scTCR contains a disulfide bond between residues of the single amino acid strand, which, in some cases, can promote stability of the pairing between the α and β regions of the single chain molecule (see e.g. U.S. Pat. No. 7,569,664). In some embodiments, the scTCR contains a covalent disulfide bond linking a residue of the immunoglobulin region of the constant domain of the α chain to a residue of the immunoglobulin region of the constant domain of the β chain of the single chain molecule. In some embodiments, the disulfide bond corresponds to the native disulfide bond present in a native dTCR. In some embodiments, the disulfide bond in a native TCR is not present. In some embodiments, the disulfide bond is an introduced non-native disulfide bond, for example, by incorporating one or more cysteines into the constant region extracellular sequences of the first and second chain regions of the scTCR polypeptide. Exemplary cysteine mutations include any as described above. In some cases, both a native and a non-native disulfide bond may be present.

In some embodiments, any of the TCRs, including a dTCR or scTCR, can be linked to signaling domains that yield an active TCR on the surface of a T cell. In some embodiments, the TCR is expressed on the surface of cells. In some embodiments, the TCR does contain a sequence corresponding to a transmembrane sequence. In some embodiments, the transmembrane domain can be a Ca or CP transmembrane domain. In some embodiments, the transmembrane domain can be from a non-TCR origin, for example, a transmembrane region from CD3z, CD28 or B7.1. In some embodiments, the TCR does contain a sequence corresponding to cytoplasmic sequences. In some embodiments, the TCR contains a CD3z signaling domain. In some embodiments, the TCR is capable of forming a TCR complex with CD3. In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal.

In some embodiments, the TCR comprises affinity to a target antigen on a target cell. The target antigen may include any type of protein, or epitope thereof, associated with the target cell. For example, the TCR may comprise affinity to a target antigen on a target cell that indicates a particular disease state of the target cell. In some embodiments, the target antigen is processed and presented by MHCs.

In one embodiment, the target cell antigen is a New York esophageal-1 (NY-ESO-1) peptide. NY-ESO-1 belongs to the cancer-testis (CT) antigen group of proteins. NY-ESO-1 is a highly immunogenic antigen in vitro and is presented to T cells via the MHC. CTLs recognizing the A2 presented epitope NY-ESO157-165, SLLMWITQC (SEQ ID NO:33), have been grown from the blood and lymph nodes of myeloma patients. T cell clones specific for this epitope have been shown to kill tumor cells. A high affinity TCR recognizing the NY-ESO₁₅₇₋₁₆₅ epitope may recognize HLA-A2-positive, NY-ESO-1 positive cell lines (but not to cells that lack either HLA-A2 or NY-ESO). Accordingly, a TCR of the present disclosure may be a HLA-A2-restricted NY-ESO-1 (SLLMWITQC; SEQ ID NO:33)-specific TCR. In one embodiment, an NY-ESO-1 TCR of the present disclosure is a wild-type NY-ESO-1 TCR. A wild-type NY-ESO-1 TCR may include, without limitation, the 8F NY-ESO-1 TCR (also referred to herein as “8F” or “8F TCR”), and the 1G4 NY-ESO-1 TCR (also referred to herein as “1G4” or “1G4 TCR”). In one embodiment, an NY-ESO-1 TCR of the present disclosure is an affinity enhanced 1G4 TCR, also called Ly95. 1G4 TCR and affinity enhanced 1G4 TCR is described in U.S. Pat. No. 8,143,376. This should not be construed as limiting in any way, as a TCR having affinity for any target antigen is suitable for use in a composition or method of the present disclosure.

### E. Methods of Producing Genetically Modified Immune Cells

The present disclosure provides methods for producing or generating the modified immune cells or precursors thereof (e.g., a T cell) disclosed herein for tumor immunotherapy, e.g., adoptive immunotherapy. The cells generally are engineered by introducing one or more genetically engineered nucleic acids encoding a CAR and/or TCR. In some embodiments, the cells also are introduced, either simultaneously or sequentially with a nucleic acid encoding the CAR and/or TCR, with an agent (e.g. Cas9/gRNA RNP) that is capable of disrupting a targeted gene (e.g., a gene encoding PRDM1, NR4A3, and/or TGFβRII).

In certain embodiments, the disclosure provides a method for generating a modified immune cell or precursor cell thereof, comprising introducing into an immune or precursor cell a CRISPR system comprising one or more polypeptides and/or nucleic acids capable of downregulating gene expression of endogenous PRDM1, NR4A3, and/or TGFβRII; and introducing into the immune or precursor cell a nucleic acid encoding an exogenous CAR, wherein the exogenous CAR comprises affinity for an antigen on a target cell.

In yet another aspect, the disclosure provides a method for generating a modified immune cell or precursor cell thereof, comprising introducing into an immune or precursor cell one or more polypeptides and/or nucleic acids capable of downregulating gene expression of endogenous PRDM1, NR4A3, and/or TGFβRII; and introducing into the immune or precursor cell a nucleic acid encoding an exogenous CAR, wherein the nucleic acid encoding an exogenous CAR is inserted into an endogenous gene locus encoding PRDM1, NR4A3, and/or TGFβRII, and wherein the exogenous CAR comprises affinity for an antigen on a target cell.

In certain embodiments, the disclosure provides a method for generating a modified immune cell or precursor cell thereof, comprising introducing into an immune or precursor cell a CRISPR system comprising one or more polypeptides and/or nucleic acids capable of downregulating gene expression of endogenous PRDM1, NR4A3, and/or TGFβRII; and introducing into the immune or precursor cell a nucleic acid encoding an exogenous TCR, wherein the exogenous TCR comprises affinity for an antigen on a target cell.

In yet another aspect, the disclosure provides a method for generating a modified immune cell or precursor cell thereof, comprising introducing into an immune or precursor cell one or more polypeptides and/or nucleic acids capable of downregulating gene expression of endogenous PRDM1, NR4A3, and/or TGFβRII; and introducing into the immune or precursor cell a nucleic acid encoding an exogenous TCR, wherein the nucleic acid encoding an exogenous TCR is inserted into an endogenous gene locus encoding PRDM1, NR4A3, and/or TGFβRII, and wherein the exogenous TCR comprises affinity for an antigen on a target cell.

A linker for use in the present disclosure allows for multiple proteins to be encoded by the same nucleic acid sequence (e.g., a multicistronic or bicistronic sequence), which are translated as a polyprotein that is dissociated into separate protein components. For example, a linker for use in a donor nucleic acid of the present disclosure comprising a nucleic acid sequence encoding a CAR and a reporter gene, allows for the CAR and the reporter gene product to be translated as a polyprotein that is dissociated into separate CAR and reporter gene product components. Various linkers that can be used are disclosed elsewhere herein, e.g., IRES, or a 2A peptide.

In some embodiments, the CAR is introduced into a cell by an expression vector. Expression vectors comprising a nucleic acid sequence encoding a CAR of the present disclosure are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Some other suitable expression vectors include Herpes simplex virus (HSV) and retrovirus expression vectors.

In certain embodiments, the nucleic acid encoding an exogenous CAR and/or TCR is introduced into the cell via viral transduction. In certain embodiments, the viral transduction comprises contacting the immune or precursor cell with a viral vector comprising the nucleic acid encoding an exogenous CAR and/or TCR. In certain embodiments, the viral vector is an adeno-associated viral (AAV) vector. In certain embodiments, the AAV vector comprises a 5′ ITR and a 3′ITR derived from AAV6. In certain embodiments, the AAV vector comprises a 5′ homology arm and a 3′ homology arm, wherein the 5′ and 3′ homology arms comprise complementarity to a target sequence in an endogenous gene locus. In certain embodiments, the AAV vector comprises a Woodchuck Hepatitis Virus post-transcriptional regulatory element (WPRE). In certain embodiments, the AAV vector comprises a polyadenylation (polyA) sequence. In certain embodiments, the polyA sequence is a bovine growth hormone (BGH) polyA sequence.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the CAR and/or TCR in the host cell. In some embodiments, the adenovirus genome is a 36 kb, linear, double stranded DNA, where a foreign DNA sequence (e.g., a nucleic acid encoding an exogenous CAR and/or TCR) may be inserted to substitute large pieces of adenoviral DNA in order to make the expression vector of the present disclosure (see, e.g., Danthinne and Imperiale, Gene Therapy (2000) 7(20): 1707-1714).

Another expression vector is based on an adeno associated virus (AAV), which takes advantage of the adenovirus coupled systems. This AAV expression vector has a high frequency of integration into the host genome. It can infect nondividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retroviral vector is constructed by inserting a nucleic acid (e.g., a nucleic acid encoding an exogenous CAR and/or TCR) into the viral genome at certain locations to produce a virus that is replication defective. Though the retroviral vectors are able to infect a broad variety of cell types, integration and stable expression of the CAR requires the division of host cells.

Lentiviral vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are capable of infecting non-dividing cells and can be used for both in vivo and ex vivo gene transfer and expression, e.g., of a nucleic acid encoding a CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell an immune cell or precursor thereof, e.g., a T cell, an NK cell, or an NKT cell.

The present disclosure also provides genetically engineered cells which include and stably express a CAR of the present disclosure. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In certain embodiments, the genetically engineered cells are autologous cells. In certain embodiments, the modified cell is resistant to T cell exhaustion.

Modified cells (e.g., comprising a CAR) may be produced by stably transfecting host cells with an expression vector including a nucleic acid of the present disclosure. Additional methods for generating a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a CAR of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). Compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the nucleic acids in the host cell, a variety of assays may be performed. Such assays include, for example, molecular biology assays well known to those of skill in the art, such as Southern and Northern blotting, RT-PCR and PCR; biochemistry assays, such as detecting the presence or absence of a particular peptide, e.g., by immunological means (ELISAs and Western blots) or by assays described herein to identify agents falling within the scope of the disclosure.

In one embodiment, the nucleic acids introduced into the host cell are RNA. In another embodiment, the RNA is mRNA that comprises in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenbom and Mierendorf, Nuc Acids Res., 13:6223-36 (1985); Nacheva and Berzal-Herranz, Eur. J. Biochem., 270:1485-65 (2003).

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

In some embodiments, a nucleic acid encoding a CAR of the present disclosure will be RNA, e.g., in vitro synthesized RNA. Methods for in vitro synthesis of RNA are known in the art; any known method can be used to synthesize RNA comprising a sequence encoding a CAR. Methods for introducing RNA into a host cell are known in the art. See, e.g., Zhao et al. Cancer Res. (2010) 15: 9053. Introducing RNA comprising a nucleotide sequence encoding a CAR into a host cell can be carried out in vitro, ex vivo or in vivo. or example, a host cell (e.g., an NK cell, a cytotoxic T lymphocyte, etc.) can be electroporated in vitro or ex vivo with RNA comprising a nucleotide sequence encoding a CAR.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the disclosure is that RNA transfection is essentially transient and a vector-free. An RNA transgene can be delivered to a lymphocyte and expressed therein following a brief in vitro cell activation, as a minimal expressing cassette without the need for any additional viral sequences. nder these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the immune cells (e.g. T cells) can be incubated or cultivated prior to, during and/or subsequent to introducing the nucleic acid molecule encoding the CAR and/or TCR and the gene editing agent (e.g. Cas9/gRNA RNP). In some embodiments, the cells (e.g. T cells) can be incubated or cultivated prior to, during or subsequent to the introduction of the nucleic acid molecule encoding the exogenous receptor (e.g. CAR and/or TCR), such as prior to, during or subsequent to the transduction of the cells with a viral vector (e.g. lentiviral vector) encoding the exogenous receptor (e.g. CAR and/or TCR). In some embodiments, the cells (e.g. T cells) can be incubated or cultivated prior to, during or subsequent to the introduction of the gene editing agent (e.g. Cas9/gRNA RNP), such as prior to, during or subsequent to contacting the cells with the agent or prior to, during or subsequent to delivering the agent into the cells, e.g. via electroporation. In some embodiments, the incubation can be both in the context of introducing the nucleic acid molecule encoding the exogenous receptor (e.g. CAR and/or TCR) and introducing the gene editing agent, e.g. Cas9/gRNA RNP. In some embodiments, the method includes activating or stimulating cells with a stimulating or activating agent (e.g. anti-CD3/anti-CD28 antibodies) prior to introducing the nucleic acid molecule encoding the exogenous receptor (e.g. CAR and/or TCR) and the gene editing agent, e.g. Cas9/gRNA RNP.

In some embodiments, introducing the gene editing agent, e.g. Cas9/gRNA RNP, is done after introducing the nucleic acid molecule encoding the exogenous receptor (e.g. CAR and/or TCR). In some embodiments, prior to the introducing of the agent, the cells are allowed to rest, e.g. by removal of any stimulating or activating agent. In some embodiments, prior to introducing the agent, the stimulating or activating agent and/or cytokines are not removed. Those of skill in the art will be able to determine the order in which each of the one or more nucleic acid sequences are introduced into the host cell.

### F. Nucleic Acids and Expression Vectors

The present disclosure provides nucleic acids encoding CARs. In one embodiment, a nucleic acid of the present disclosure comprises a nucleic acid sequence encoding an exogenous CAR (e.g., a PSMA CAR). Also provided are nucleic acids encoding TCRs. In some embodiments, a nucleic acid of the present disclosure is provided for the production of a CAR and/or TCR as described herein, e.g., in a mammalian cell. In some embodiments, a nucleic acid of the present disclosure provides for amplification of the CAR-encoding or TCR-encoding nucleic acid.

A linker for use in the present disclosure allows for multiple proteins to be encoded by the same nucleic acid sequence (e.g., a multicistronic or bicistronic sequence), which are translated as a polyprotein that is dissociated into separate protein components. In some embodiments, the linker comprises a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in the present disclosure.

In some embodiments, the linker comprises a nucleic acid sequence that encodes for a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide for use in the present disclosure.

In some embodiments, a linker further comprises a nucleic acid sequence that encodes a furin cleavage site. Furin is a ubiquitously expressed protease that resides in the trans-golgi and processes protein precursors before their secretion. Furin cleaves at the COOH— terminus of its consensus recognition sequence. Various furin consensus recognition sequences (or “furin cleavage sites”) are known to those of skill in the art. Those of skill in the art would be able to select the appropriate Furin cleavage site for use in the present disclosure.

In some embodiments, the linker comprises a nucleic acid sequence encoding a combination of a Furin cleavage site and a 2A peptide. Examples include, without limitation, a linker comprising a nucleic acid sequence encoding Furin and F2A, a linker comprising a nucleic acid sequence encoding Furin and E2A, a linker comprising a nucleic acid sequence encoding Furin and P2A, a linker comprising a nucleic acid sequence encoding Furin and T2A. Those of skill in the art would be able to select the appropriate combination for use in the present disclosure. In such embodiments, the linker may further comprise a spacer sequence between the Furin and 2A peptide. Various spacer sequences are known in the art, including, without limitation, glycine serine (GS) spacers such as (GS)n, (GSGGS)n (SEQ ID NO:9) and (GGGS)n (SEQ ID NO:10), where n represents an integer of at least 1. Exemplary spacer sequences can comprise amino acid sequences including, without limitation, GGSG (SEQ ID NO:12), GGSGG (SEQ ID NO:13), GSGSG (SEQ ID NO: 14), GSGGG (SEQ ID NO:15), GGGSG (SEQ ID NO:16), GSSSG (SEQ ID NO:17), and the like. Those of skill in the art would be able to select the appropriate spacer sequence for use in the present disclosure.

In some embodiments, a nucleic acid of the present disclosure may be operably linked to a transcriptional control element, e.g., a promoter, and enhancer, etc. Suitable promoter and enhancer elements are known to those of skill in the art.

In certain embodiments, the nucleic acid encoding an exogenous CAR and/or TCR is in operable linkage with a promoter. In certain embodiments, the promoter is a phosphoglycerate kinase-1 (PGK) promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is for the production of a transgenic polypeptide product that is released upon CAR signaling. See, e.g., Chmielewski and Abken, Expert Opin. Biol. Ther. (2015) 15(8): 1145-1154; and Abken, Immunotherapy (2015) 7(5): 535-544. In some embodiments, a nucleic acid of the present disclosure further comprises a nucleic acid sequence encoding a cytokine operably linked to a T-cell activation responsive promoter. In some embodiments, the cytokine operably linked to a T-cell activation responsive promoter is present on a separate nucleic acid sequence. In one embodiment, the cytokine is IL-12.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in anyway as limiting: Bacterial: pBs, phagescript, PsiX174, pBluescript SK, pBs KS, pNH8a, pNH16a, pNH18a, pNH46a (Stratagene, La Jolla, Calif, USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the CAR into an immune cell or precursor thereof (e.g., a T cell). Accordingly, an expression vector (e.g., a lentiviral vector) of the present disclosure may comprise a nucleic acid encoding for a CAR. In some embodiments, the expression vector (e.g., lentiviral vector) will comprise additional elements that will aid in the functional expression of the CAR encoded therein. In some embodiments, an expression vector comprising a nucleic acid encoding for a CAR further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR encoding nucleic acid sequence). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present disclosure. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present disclosure. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present disclosure further comprises a WPRE sequence. Various posttranscriptional regulator elements are known to those of skill in the art and may be incorporated into a vector (e.g., lentiviral vector) of the present disclosure. A vector of the present disclosure may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present disclosure includes a 3′ U3 deleted LTR. Accordingly, a vector (e.g., lentiviral vector) of the present disclosure may comprise any combination of the elements described herein to enhance the efficiency of functional expression of transgenes. For example, a vector (e.g., lentiviral vector) of the present disclosure may comprise a WPRE sequence, cPPT sequence, RRE sequence, 5′LTR, 3′ U3 deleted LTR′ in addition to a nucleic acid encoding for a CAR.

Vectors of the present disclosure may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present disclosure may be RNA, e.g., in vitro synthesized RNA. Methods for in vitro synthesis of RNA are known to those of skill in the art; any known method can be used to synthesize RNA comprising a sequence encoding a CAR of the present disclosure. Methods for introducing RNA into a host cell are known in the art. See, e.g., Zhao et al. Cancer Res. (2010) 15: 9053. Introducing RNA comprising a nucleotide sequence encoding a CAR of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a CAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

### G. Methods of Treatment

The modified cells (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the disclosure provides a method for adoptive cell transfer therapy comprising administering to a subject in need thereof a modified T cell of the present invention. In another aspect, the disclosure provides a method of treating a disease or condition in a subject comprising administering to a subject in need thereof a population of modified T cells.

Also included is a method of treating a disease or condition in a subject in need thereof comprising administering to the subject a genetically edited modified cell (e.g., genetically edited modified T cell). In one embodiment, the method of treating a disease or condition in a subject in need thereof comprises administering to the subject a genetically edited modified cell (e.g. comprising downregulated expression of endogenous PRDM1, NR4A3, and/or TGFβRII) comprising an exogenous CAR and/or TCR.

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells disclosed herein can be administered to an animal, preferably a mammal, even more preferably a human, to treat a cancer. In addition, the cells disclosed herein can be used for the treatment of any condition related to a cancer, especially a cell-mediated immune response against a tumor cell(s), where it is desirable to treat or alleviate the disease. The types of cancers to be treated with the modified cells or pharmaceutical compositions of the disclosure include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a solid tumor or a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

In certain exemplary embodiments, the modified immune cells of the disclosure are used to treat a myeloma, or a condition related to myeloma. Examples of myeloma or conditions related thereto include, without limitation, light chain myeloma, non-secretory myeloma, monoclonal gamopathy of undertermined significance (MGUS), plasmacytoma (e.g., solitary, multiple solitary, extramedullary plasmacytoma), amyloidosis, and multiple myeloma. In one embodiment, a method of the present disclosure is used to treat multiple myeloma. In one embodiment, a method of the present disclosure is used to treat refractory myeloma. In one embodiment, a method of the present disclosure is used to treat relapsed myeloma.

In certain exemplary embodiments, the modified immune cells of the disclosure are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In one embodiment, a method of the present disclosure is used to treat cutaneous melanoma. In one embodiment, a method of the present disclosure is used to treat refractory melanoma. In one embodiment, a method of the present disclosure is used to treat relapsed melanoma.

In yet other exemplary embodiments, the modified immune cells of the disclosure are used to treat a sarcoma, or a condition related to sarcoma. Examples of sarcoma or conditions related thereto include, without limitation, angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, and synovial sarcoma. In one embodiment, a method of the present disclosure is used to treat synovial sarcoma. In one embodiment, a method of the present disclosure is used to treat liposarcoma such as myxoid/round cell liposarcoma, differentiated/dedifferentiated liposarcoma, and pleomorphic liposarcoma. In one embodiment, a method of the present disclosure is used to treat myxoid/round cell liposarcoma. In one embodiment, a method of the present disclosure is used to treat a refractory sarcoma. In one embodiment, a method of the present disclosure is used to treat a relapsed sarcoma.

The cells to be administered may be autologous, with respect to the subject undergoing therapy.

The administration of the cells may be carried out in any convenient manner known to those of skill in the art. The cells of the present disclosure may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells are injected directly into a site of inflammation in the subject, a local disease site in the subject, alymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8⁺ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells disclosed herein (e.g., a modified cell comprising a CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MED14736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell of the present disclosure.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the disclosure can be administered in dosages and routes and at times to be determined in appropriate pre-clinical and clinical experimentation and trials. Cell compositions may be administered multiple times at dosages within these ranges. Administration of the cells of the disclosure may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the disclosure provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

The modified immune cells comprising an exogenous CAR and/or TCR of the present disclosure may be used in a method of treatment as described herein. In some embodiments, the modified immune cells comprise an insertion and/or deletion in a PRDM1, NR4A3, and/or TGFβRII gene locus that is capable of downregulating gene expression of PRDM1, NR4A3, and/or TGFβRII. In some embodiments, when PRDM1, NR4A3, and/or TGFβRII is downregulated, the function of the immune cell comprising an exogenous CAR and/or TCR is enhanced. For example, without limitation, when downregulated, PRDM1, NR4A3, and/or TGFβRII enhances tumor infiltration, tumor killing, and/or resistance to immunosuppression of the immune cell comprising an exogenous CAR and/or TCR. In some embodiments, when PRDM1, NR4A3, and/or TGFβRII is downregulated, T cell exhaustion is reduced or eliminated.

As such, the modified immune cells comprising an exogenous CAR and/or TCR of the present disclosure when used in a method of treatment as described herein, enhances the ability of the modified immune cells in carrying out their function. Accordingly, the present disclosure provides a method for enhancing a function of a modified immune cell for use in a method of treatment as described herein.

In one aspect, the disclosure includes a method of treating cancer in a subject in need thereof, comprising administering to the subject any one of the modified immune or precursor cells disclosed herein. Yet another aspect of the disclosure includes a method of treating cancer in a subject in need thereof, comprising administering to the subject a modified immune or precursor cell generated by any one of the methods disclosed herein.

Still another aspect of the disclosure includes a method of treating a disease or disorder in a subject in need thereof, comprising administering to the subject a modified T cell comprising: a modification (e.g., CRISPR-mediated modification) in an endogenous gene locus encoding PRDM1, wherein the modification is capable of downregulating gene expression of, or knocking out, endogenous PRDM1; and an exogenous CAR and/or TCR comprising affinity for an antigen on a target cell. Another aspect of the disclosure includes a method of treating a disease or disorder in a subject in need thereof, comprising administering to the subject a modified T cell comprising: a modification (e.g., CRISPR-mediated modification) in an endogenous gene locus encoding PRDM1, wherein the modification is capable of downregulating gene expression of, or knocking out, endogenous PRDM1, a modification (e.g., CRISPR-mediated modification) in an endogenous gene locus encoding NR4A3, wherein the modification is capable of downregulating gene expression of, or knocking out, endogenous NR4A3; and an exogenous CAR and/or TCR comprising affinity for an antigen on a target cell. Another aspect of the disclosure includes a method of treating a disease or disorder in a subject in need thereof, comprising administering to the subject a modified T cell comprising: a modification (e.g., CRISPR-mediated modification) in an endogenous gene locus encoding PRDM1, wherein the modification is capable of downregulating gene expression of, or knocking out, endogenous PRDM1, a modification (e.g., CRISPR-mediated modification) in an endogenous gene locus encoding TGFβRII, wherein the modification is capable of downregulating gene expression of, or knocking out, endogenous TGFβRII; and an exogenous CAR and/or TCR comprising affinity for an antigen on a target cell.

### H. Sources of Immune Cells

In certain embodiments, a source of immune cells is obtained from a subject for ex vivo manipulation. Sources of target cells for ex vivo manipulation may also include, e.g., autologous or heterologous donor blood, cord blood, or bone marrow. For example the source of immune cells may be from the subject to be treated with the modified immune cells disclosed herein, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker−) or express relatively low levels (marke^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD 14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA 5 and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L- and CD45RO. In one example, to enrich for CD4+ cells by negative selection, a monoclonal antibody cocktail typically includes antibodies to CD14, CD20, CDl lb, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### I. Expansion of Immune Cells

Whether prior to or after modification of cells to express a CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the disclosure may be expanded by contact with a surface having attached thereto an agent that stimulates a CD3/TCR complex associated signal and a ligand that stimulates a co-stimulatory molecule on the surface of the T cells. In particular, T cell populations may be stimulated by contact 10 with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. he T cells are then harvested from the culture apparatus whereupon the T cells can be used immediately or cryopreserved to be stored for use at a later time. In one embodiment, the disclosure includes cryopreserving the expanded T cells. The cryopreserved T cells are thawed prior to introducing nucleic acids into the T cell.

In another embodiment, the method comprises isolating T cells and expanding the T cells. In another embodiment, the disclosure further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic 10 acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### J. Pharmaceutical Compositions and Formulations

Also provided are populations of immune cells described herein, compositions containing such cells and/or enriched for such cells, such as in which cells expressing CAR make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.00010% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

Cell Lines: PSMA-positive PC3 and NALM6 cells that are engineered to express click beetle green luciferase and green fluorescent protein (CBG-GFP), were kindly provided by Carl H. June and Marco Ruella, respectively. PC3-PSMA cells were cultured in Dulbecco's Modified Eagle Medium (DMEM) supplemented with 10% fetus bovine serum (FBS) and streptomycin/penicillin. The NALM6 cells were maintained in Roswell Park Memorial Institute (RPMI) 1640 media supplemented with 10% fetus bovine serum (FBS) and streptomycin/penicillin (R10 media). HEK 293T cells, used for lentivirus production, were obtained from ATCC and cultured in R10 media.

Lentivirus production: Vector construction and lentiviral production were conducted as previously described (Kloss et al. Molecular Therapy. 2018; 26:1855-66). In brief, CARs comprised of anti-PSMA (Kloss et al., Molecular Therapy 26, 1855-1866 (2018)), anti-CD19 (Milone et al. Mol Ther 17, 1453-1464 (2009)), or anti-Mesothelin (Mol Ther 27, 1919-1929 (2019)) single-chain variable fragments (scFv) fused to 4-1BB and CD3 stimulatory endodomains were subcloned into the pTRPE vector. Lentivirus supernatant was collected from 293T cells transfected with the pTRPE transfer vector and packaging plasmids using Lipofectamine 2000 (Thermo Scientific) and concentrated using ultracentrifugation.

T-cell culture and lentiviral transduction: Normal donor T-cells were isolated from peripheral blood mononuclear cells (PBMC) using pan T cell isolation kit. Cells were activated with CD3/CD28 coated Dynabeads (Gibco) at 3:1 beads:cell ratio in T-cell media (OpTmizer CTS SFM media (Gibco) supplemented with 5% human AB serum and 100 u/mL human IL-2). Following a 24-hour incubation, lentivirus encoding the PSMA CAR was added to the culture.

CRISPR/Cas9-mediated knockout: Beads were removed using a magnet on day 3 and electroporation was carried out using an P3 primary cell 4D-nucleofector kit (Lonza). 2×10⁶ CAR T-cells were transfected with 12 μg TrueCut™ S. pyogenes Cas9 (Invitrogen) and 0.2 nmol chemically-modified tracrRNA and crRNA (IntegratedDNA Technologies) with program EO-115. Following electroporation, CAR T-cells were cultured in T-cell media. The crRNA sequences used in this studies were: AAVS1: 5′-CCATCGTAAGCAAACCTTAG-3′ (SEQ ID NO: 1), PRDM1: 5′-CATCAGCACCAGAATCCCAG-3′(SEQ ID NO: 2), TCF7: 5′-TCAGGGAGTAGAAGCCAGAG-3′(SEQ ID NO: 3), NR4A3: 5′-CCTTGGCAGCACTGAGATCA-3′(SEQ ID NO: 4). The frequency of targeted mutations generated by double strand break were determined by amplicons seq and TIDE (tracking of indels by decomposition) analysis. Primers used in targeted amplification were: PRDM1-F1: tctcagaaggagccacaggaacgg (SEQ ID NO: 5), PRDM1-R1: cacccaccctatgctgcaagttgc (SEQ ID NO: 6), NR4A3-F1: gaggagaggatgacacttcctctctgtttc (SEQ ID NO: 7), NR4A3: ctgcccagcacctccatgtacttcaagcag (SEQ ID NO: 8). Western blot and flow cytometric analysis were conducted to confirm knockout at the protein level.

Flow Cytometry: Surface anti-human antibodies were stained in FACS buffer (PBS+2% FBS). Expression of PSMA CARs was detected using APC-conjugated PSMA (Sino Biological). T-cell immunophenotype was examined using following antibodies: PD1-BV421 (Biolegend #329920), CD45-BV570 (Biolegend #304226), CD8-BV650 (Biolegend #301042), CD8-APC-H7 (BD biosciences #560179), CD4-BV785 (Biolegend #317442), TIM3-PE (Biolegend #345006), CCR7-PE-CF594 (BD biosciences #562381), CD62L-PE-Cy5 (Biolegend #304808), LAG3-PE-Cy7(eBioscience #25-2239-42), hCD45-APC (BD biosciences #340943), murine CD45-PerCP-Cy5.5 (Biolegend #103132), CD127-BV570 (Biolegend #351307), HLA-DR-Alexa Fluor700 (Biolegend #307625), CD25-APC (ebioscience #17-0259-42). For intracellular staining, cells were permeabilized and washed using Foxp3 transcription factor staining buffer kit (eBioscience) and stained with following antibodies: IL2-PE-CF594 (BD biosciences #562384), IFNγ-BV570 (Biolegend #502534), TNFα-Alexa Fluor700 (Biolegend #502928), Perforin-BV421 (Biolegend #353307), Perforin-APC (Biolegend #308112), GZMB-PE-Cy5.5 (Invitrogen #GRB18), TCF1-Alexa Fluor488 (Cell signaling technology #6444S), TOX-APC (Miltenyi Biotech #130-107-785), NFATC1-PE (Biolegend #649606).

Restimulation assay: CAR-T expansion capacity and effector function during chronic CAR-T activation were assessed using a restimulation assay as previously described (Kloss et al., Molecular Therapy. 2018; 26:1855-66). Briefly, AAVS1 KO, PRDM1 KO, TCF7 KO, NR4A3 KO, PRDM1+TCF7 dKO, PRDM1+NR4A3 dKO PSMA CAR-positive T-cells were isolated using Biotin-goat anti-mouse IgG F(ab)2 fragment (Jackson ImmunoResearch #115-065-072) and anti-biotin Kits (Miltenyi Biotech). Following the isolation, purity of CAR T-cells was confirmed by flow cytometric analysis and CAR T-cells were serially exposed (every 2-5 days) to irradiated PC3-PSMA cells at an effector-to-target (E:T) ratio of 3:1 or 1:1. Supernatants were collected 24 hours post tumor challenge for cytokine analysis using Legendplex human CD8 panel (Biolegend), and the number of T-cells in the culture was monitored using a Luna automated cell counter (Logos Biosystems) during the assay.

Cytotoxicity assay: The killing kinetics of engineered CAR T-cell against PC3-PSMA cells was assessed using the xCELLigence system (ACEA Biosciences Inc.). CAR-positive T-cells were magnetically enriched prior to the cytotoxicity assay. 2×10⁴ PC3-PSMA cells were seeded in E-Plate VIEW 96 PET microwell plates. After 24 hours, PSMA CAR T-cells, control (un-transduced) T-cells, and 20% Tween20 were added to achieve the desired E:T ratio. Electrical impedance was monitored in 20-minute intervals over 7 days and cytotoxicity was assessed by normalized cell index value and cytolysis (%).

Quantitative real-time PCR: After 2-4 rounds of restimulation assay, CD8 CAR T-cells were isolated from the culture using CD8 microbeads (Miltenyi Biotech) for qRT-PCR. Total RNA was extracted from CD8 CAR T-cells using RNA Clean & Concentrator™ kits (Zymo Research). cDNA was synthesized using PrimeScript 1st strand cDNA Synthesis Kit (Takara Bio) following manufacturer's protocol and qRT-PCR was conducted with QuantStudio3 (Applied Biosystems) using Applied Biosystems TaqMan Fast Advanced Master Mix (Thermo Scientific). The primer/probe set (Thermofisher Scientific #4453320) used in this study were: NR4A2: Hs00428691_ml, NR4A3: Hs00545009_gl.

Western blot analysis: The T-cells (1×10⁶ cells) were suspended in a low-salt lysis buffer (10 mM HEPES, pH 7.9, 10 mM KCl, 0.1 mM EDTA, 0.1 mM EGTA, 1 mM DTT, 0.5 mM PMSF, 2 μg/ml aprotinin, 2 μg/ml leupeptin) and allowed to swell on ice for 30 min. The tissues were then homogenized using a Polytron homogenizer (Thermo fisher scientific). After centrifugation (1000×g), supernatants obtained from tissue homogenatesor cells lysis (30 μg) were analyzed by 10% SDS-polyacrylamide gel electrophoresis and electrophoretically transferred to PVDF membranes (Millipore). The membranes were washed with PBS containing 0.1% Tween 20 (PBST), and then blocked for 1 h in 5% skim milk in PBST. After the membranes were washed with PBST, Membranes were incubated for overnight with one of the following antibodies: Rabbit monoclonal anti-Blimp1 (Cell signaling #9115; 1:100), Mouse monoclonal anti-NR4A3 (Sigma-Aldrich #SAB1404566; 1:100). Membranes were washed with PBST and treated horseradish peroxidase-coupled goat anti-mouse or anti-rabbit secondary antibodies (Thermo Fisher; 1:1000) in PBST for 1 h. After washing, the protein bands were visualized by Pierce™ ECL western blotting substrate (Thermo Fisher).

Mouse xenograft studies: Mouse studies were performed with 6- to 8-week-old male NOD/SCID/IL-2Ry-null (NSG) mice in compliance with University of Pennsylvania Institutional Animal Care and Use Committee protocol. For a CRPC xenograft model, 10⁶ or 5×10⁶ PC3-PSMA-CBG-GFP tumor cells were premixed with Matrigel mixture (1:1 in X-vivo 15 media) and subcutaneously injected into NSG mice. When the average tumor size reached 150-200 mm³ or 500 mm³, 3.5×10⁵ PSMA-CAR T-cells were injected intravenously. Tumor growth was weekly monitored by caliper measurements (tumor volume=(length×width²)/2). The animals were sacrificed when the tumor volume exceeded 1500 mm³ or exceeded 2 cm in diameter. To characterize CAR T-cell phenotype and function, peripheral blood and tumor tissues were isolated from each mouse. Peripheral blood samples were obtained at peak CAR-T expansion via cheek bleeding followed by ACK buffer (Lonza) treatment for red blood cell lysis. Tumors, isolated on day 13 post CAR-T injection, were minced with scalpel and treated with 100 U/mL collagenase IV and 0.25 mg/mL DNase I for 1 hour in 37° C. The number of human T-cells in peripheral blood and tumors was quantified using 123count eBeads (Thermo Fisher). To assess the effector function of tumor-infiltrating T cells, T-cells were stimulated with 50 ng/mL phorbol 12-myristate 13-acetate and 1 mg/mL ionomycin in the presence of 5 ug/mL Brefeldin A for 6 hours and expression of IL2, IFNγ and TNFα were assessed by intracellular staining. For B-cell acute lymphoblastic leukemia model, NSG mice were intravenously transplanted with 1×10⁶ NALM6-CBG cells. On day 7 post tumor injection, 3×10⁵ CD19 CAR T-cells were intravenously infused, and tumor growth was assessed twice a week using bioluminescent imaging. Peripheral blood was isolated on day 24 and immunophenotype was characterized using flow cytometric analysis.

Single Cell RNA-seq: UPCC32816 PSMA CAR T-cell products from 5 different subjects were thawed at 37° C. water bath and dead cells were eliminated by Dead Cell Removal kit (Miltenyi Biotec). PSMA CAR-positive T-cells were enriched by magnetic cell separation using Biotin-goat anti-mouse IgG F(ab)2 fragment (Jackson ImmunoResearch #115-065-072) and anti-biotin Kits (Miltenyi Biotech). scRNA-seq libraries were prepared using Chromium Single Cell 3′ v3.1 Reagent Kits (10× Genomics) following the manufacturer's instruction. The isolated CAR T-cells were washed and resuspended in PBS containing 0.04% BSA and ˜20,000 cells were loaded per reaction to capture ˜10,000 cells. Sequencing was performed on Novaseq 6000 system at a depth of >20,000 reads per cell.

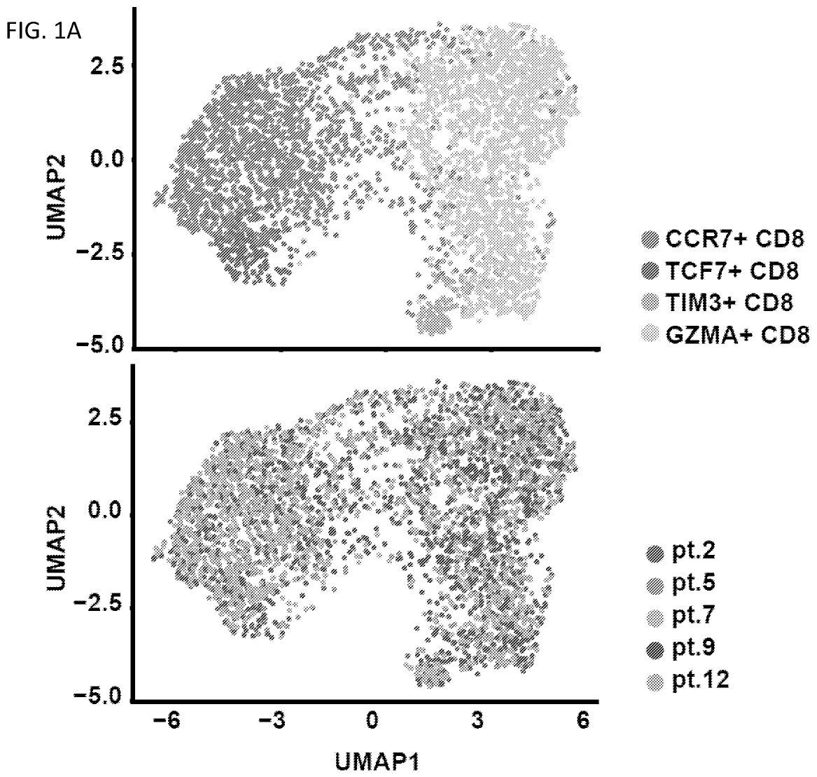

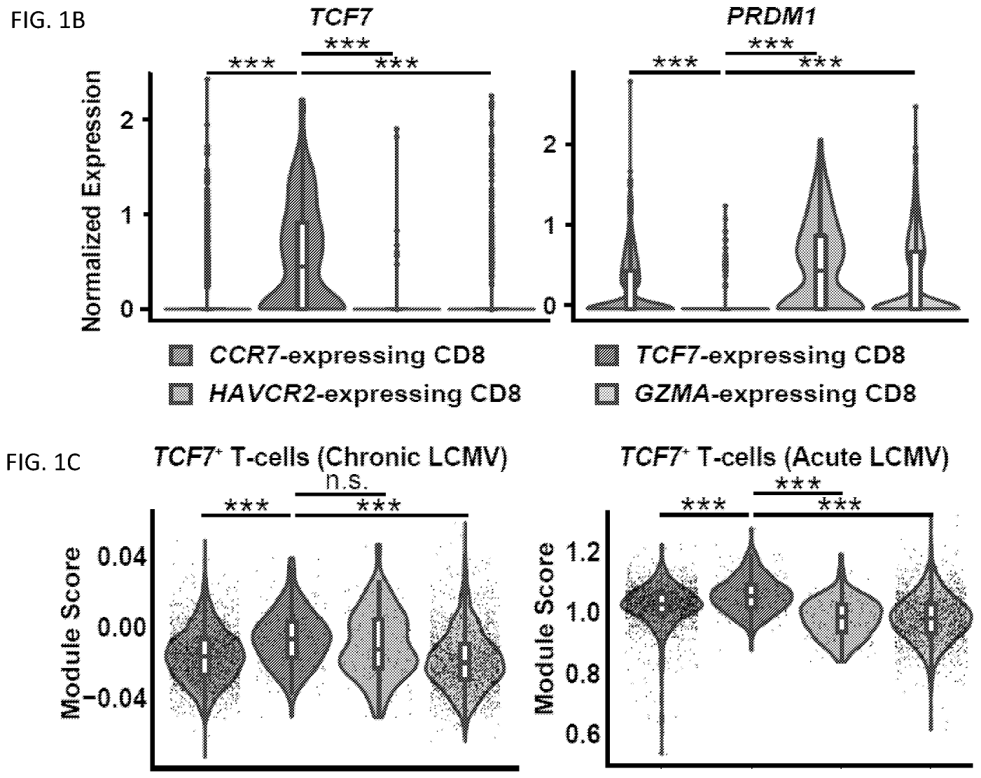

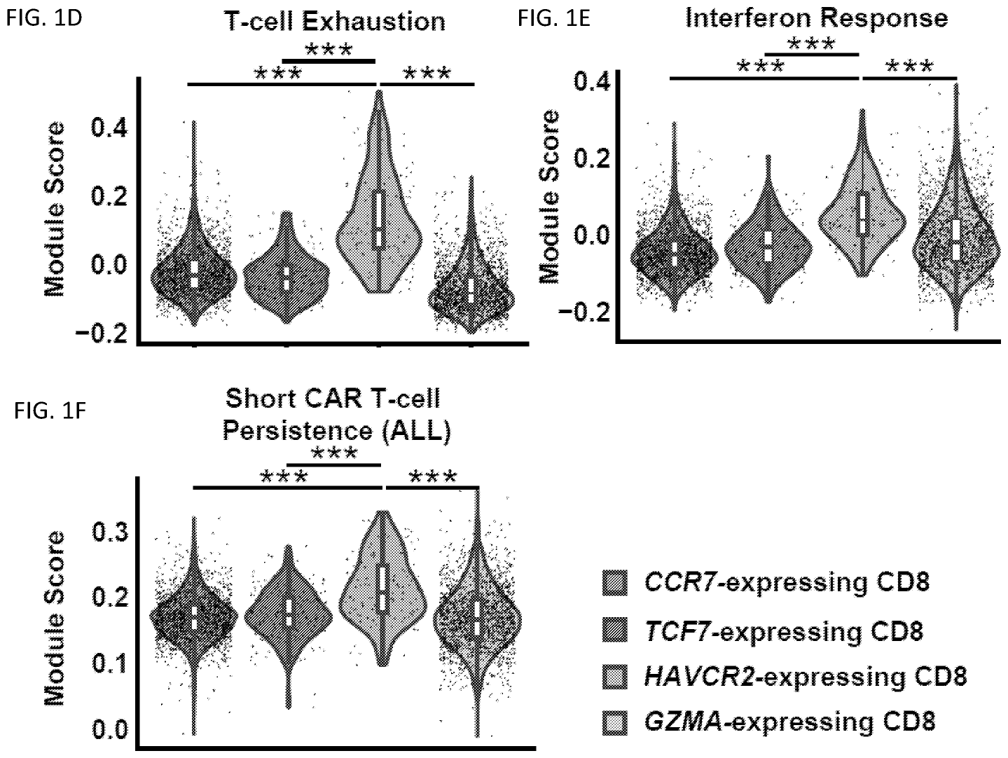

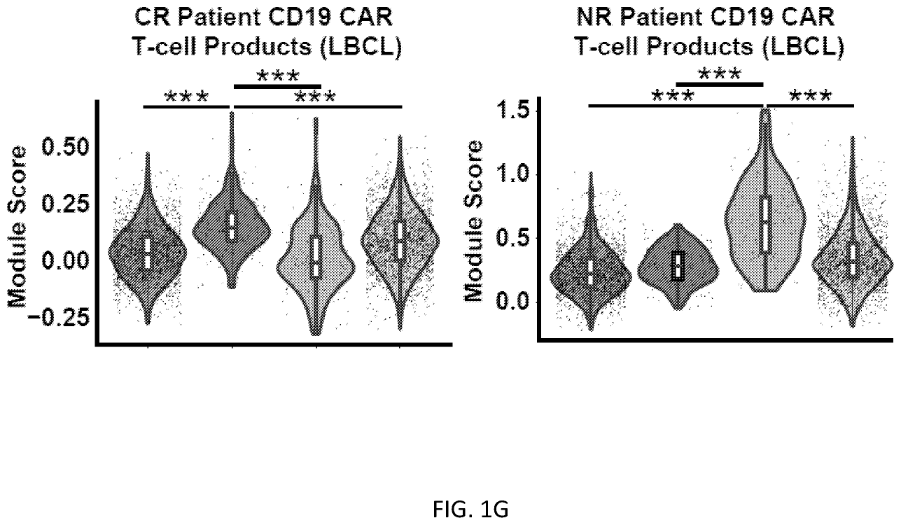

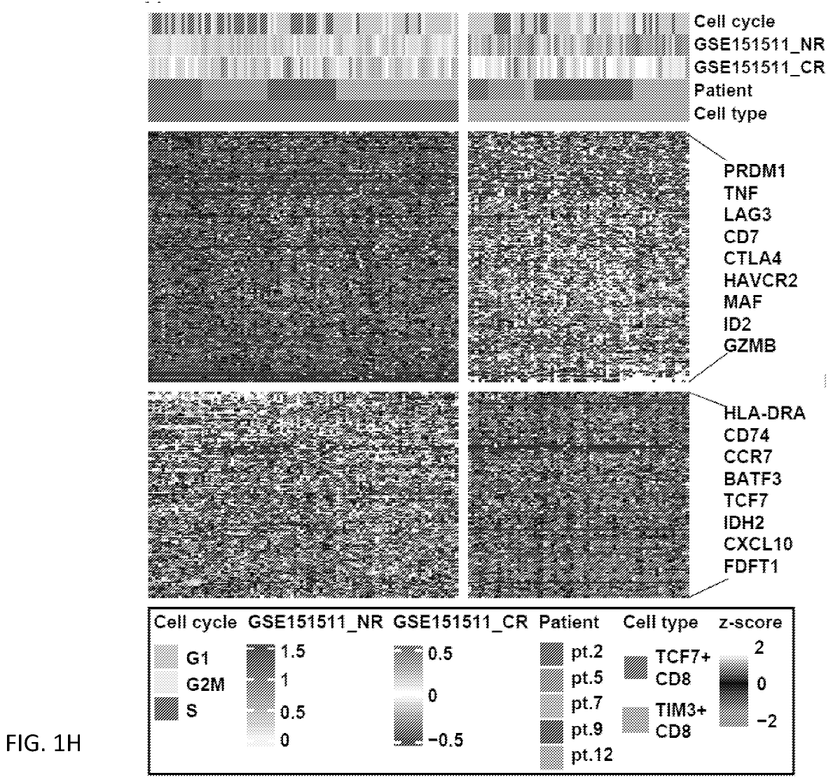

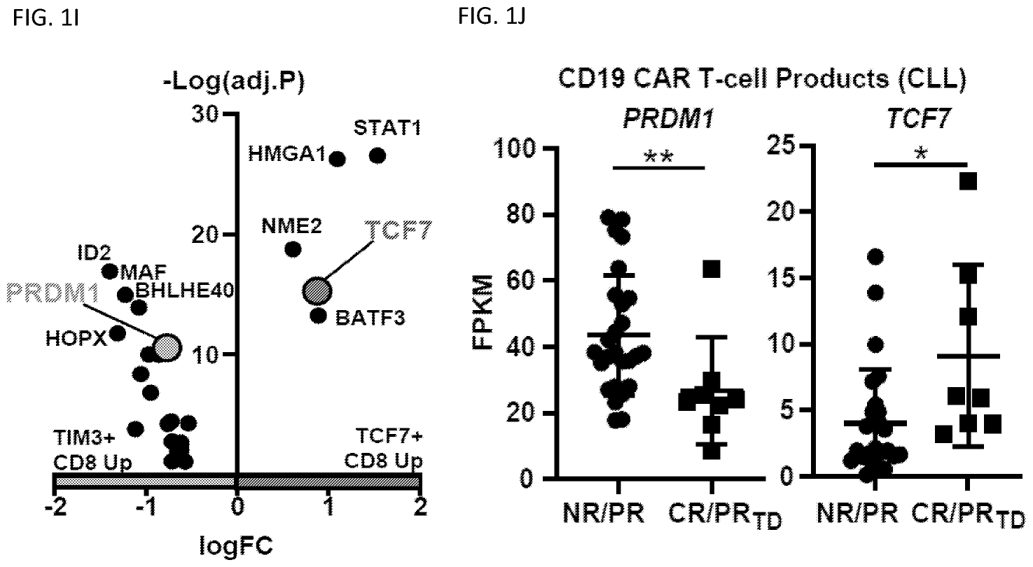

The reads were aligned to human reference genome (GRCh38) using Cell Ranger version 6.0.0. Subsequent quality control and downstream analysis were performed using Seurat 4.0. Cells were filtered based on the following criteria to eliminate low-quality cells: 1) minimum of 1000 genes and maximum of 6000 genes detected per cells 2) less than 15% of mitochondrial gene counts. After quality control, 20,702 cells were remained. The gene-cell matrices from five CAR-T products were then integrated using the SelectIntegrationFeatures, PrepSCTIntegration, FindIntegrationAnchors, and IntegrateData functions in Seurat. For FIG. 1, CD8-positive cells were clustered using FindNeighbors/FindClusters, followed differential gene expression analysis using FindClusters command. Then, module scores were calculated using AddModuleScore to assess the enrichment of gene signatures in each CD8 subcluster.

Bulk RNA-seq: On day 0, day 5, and day 20 of restimulation assay, before stimulation and after first and fourth tumor challenge respectively, CD8⁺ CAR T-cells were isolated using CD8 microbeads (Miltenyi Biotec) and total mRNA was isolated using RNA Clean & Concentrator™ kits (Zymo Research). Bulk RNA-seq was conducted by Novogene using the Novaseq6000 system with paired-end 150 bp, at 40×10⁶ reads per sample. Reads were pseudoaligned to human genome (GRCh38) transcriptomes using kallisto v0.46.0.

Differential expression analyses between AAVS1 KO PSMA CAR T-cells and PRDM1 KO PSMA CAR T-cells were performed using the edgeR v3.34.0 and limma v3.48.0 packages. Briefly, expression data were normalized using trimmed mean of log expression ratios method and transformed into log 2 (counts per million). Linear models were used to assess differential expression, and p-values were adjusted using the Benjamini and Hochberg method. Gene set enrichment analysis was conducted using the GSEABase v1.54.0, clusterProfiler v4.0.2, and msigdbr v7.4.1.

ATAC-seq: On day 0 and day 20 of the restimulation assay, dead cells were eliminated by Dead Cell Removal kit (Miltenyi Biotec) and CD8⁺ CAR T-cells were isolated using CD8 microbeads (Miltenyi Biotec). 100,000 cells per sample were cryopreserved. Library preparation and sequencing were performed by Novogene using NovaSeq 6000 (paired-end 150 bp reads) at a depth of 30×10⁶ reads per sample.

FASTQ files for each sample were trimmed of adapter contamination using cutadapt (https://github.com/marcelm/cutadapt/). They were aligned to the hg19 reference genome using Bowtie2, restricting to properly aligned and properly paired reads between 10 and 1000 base pairs. Mitochondrial reads were removed (https://github.com/jsh58/harvard/blob/master/removeChrom.py). Files were sorted using samtools, and PCR duplicates were removed using Picard. BAM files were indexed using samtools in order to visualize tracks in IGV. Peak calling was performed with MACS2 with an FDR q-value of 0.01. The R package Diffbind was used to remove ENCODE blacklisted regions (https://sites.google.com/site/anshulkundaje/projects/blacklists), then to identify peaks differentially opened between the control and the PRDM1 knockout. The findMotifsGenome script from HOMER was used to map the hg19 genome for occurrences of the PRDM1 motif (derived from ENCODE data accessible via GEO at GSE31477) and the NFACT1 motif (Kivioja et al., Genome Res. 2010; 20:861-73).

Statistical Analysis: Statistical analyses were conducted using Shapiro-Wilk test and D'Agostino & Pearson test for normality tests. Comparison of the two groups was performed using Mann Whitney U test and Student's t-test as appropriate for paired or unpaired samples. For nonparametric comparison of three or more groups in FIG. A, Kruskal-Wallis test with a post hoc Dunn's multiple comparison test was used. Mouse survival was assessed using Gehan-Breslow-Wilcoxon test. Statistical tests were performed using Prism 9 and p-values <0.05 were considered significant.

### Example 1: TCF7T CD8 and TIM3⁺ CD8 Populations within the Infusion Product are Associated with Favorable and Poor Clinical Response, Respectively

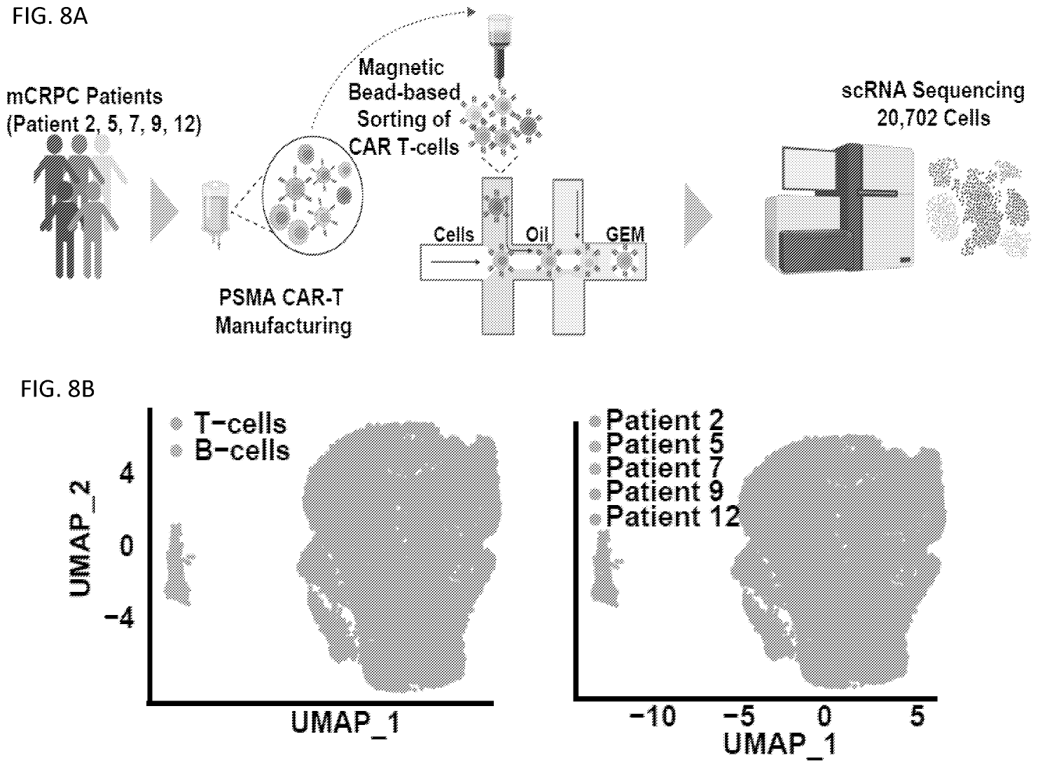

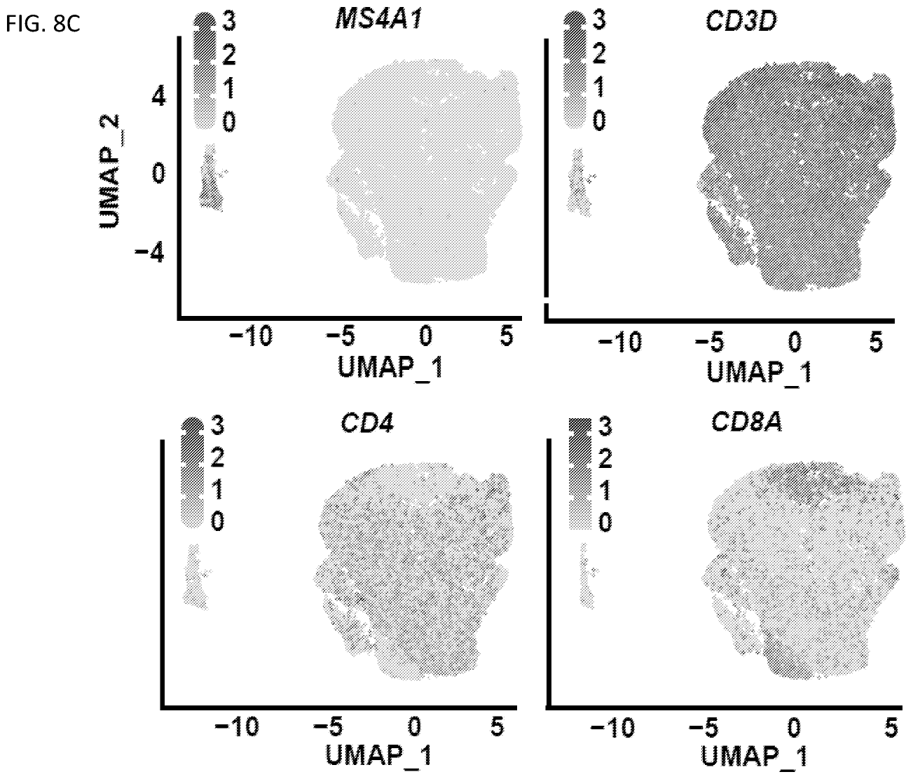

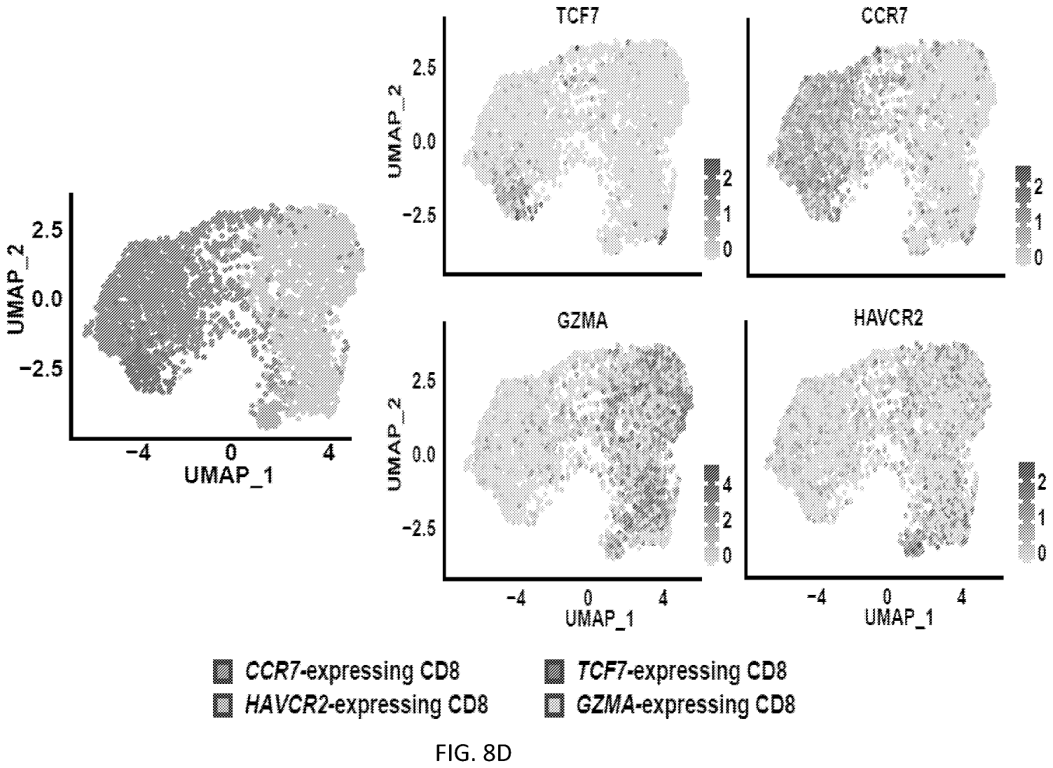

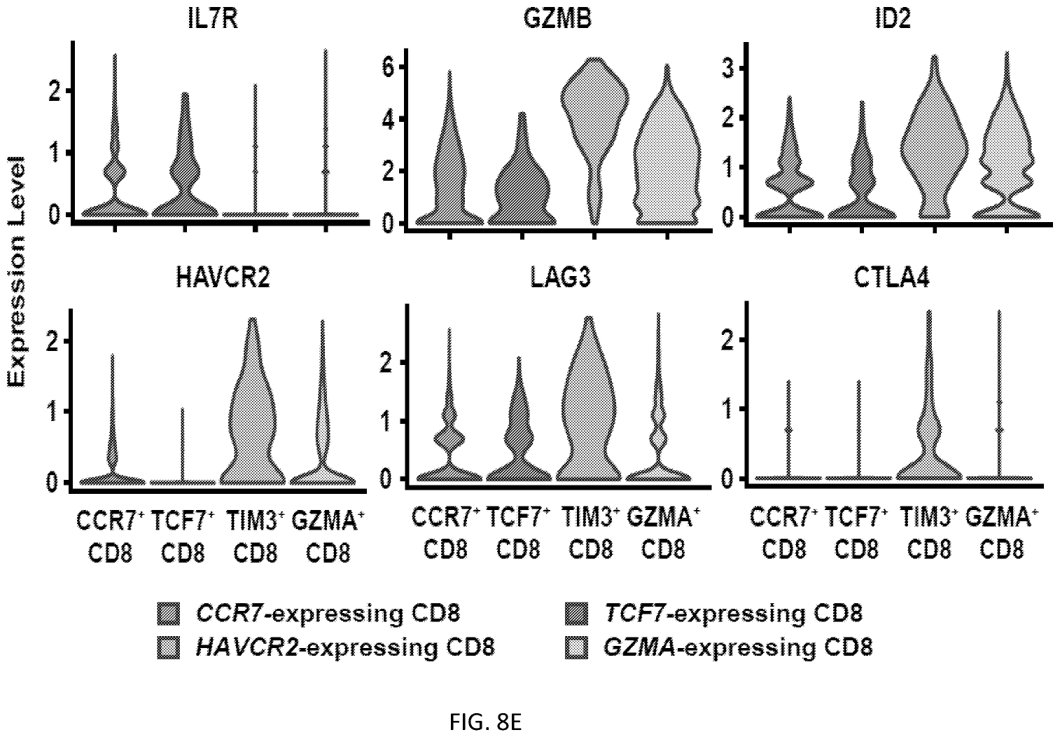

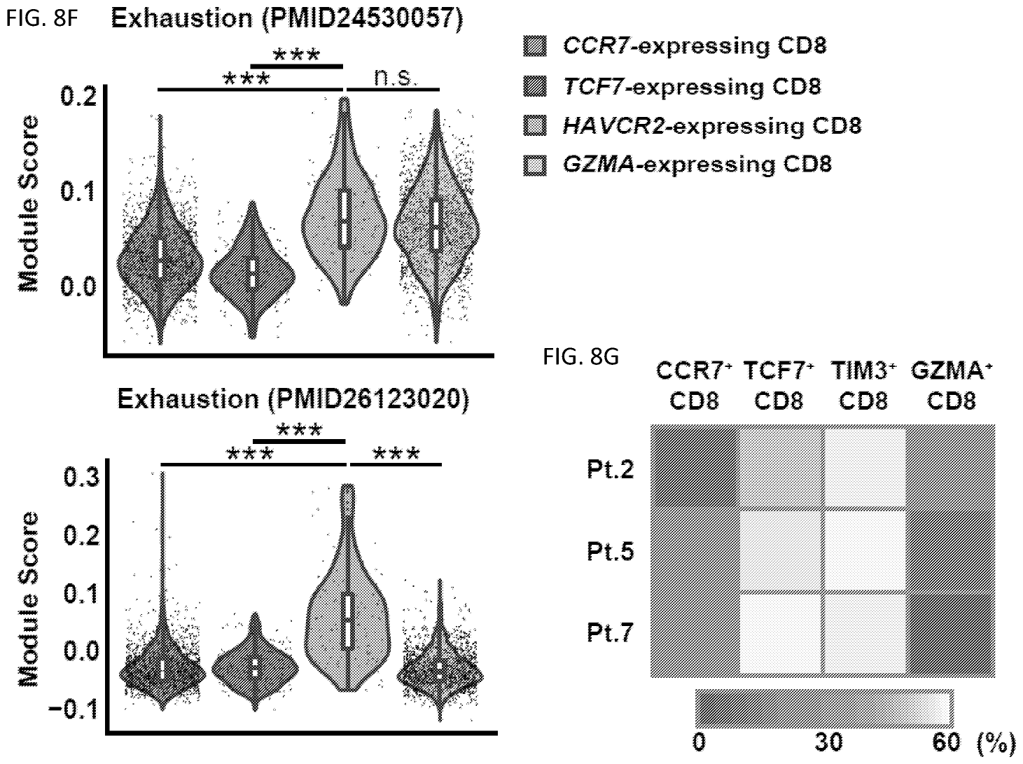

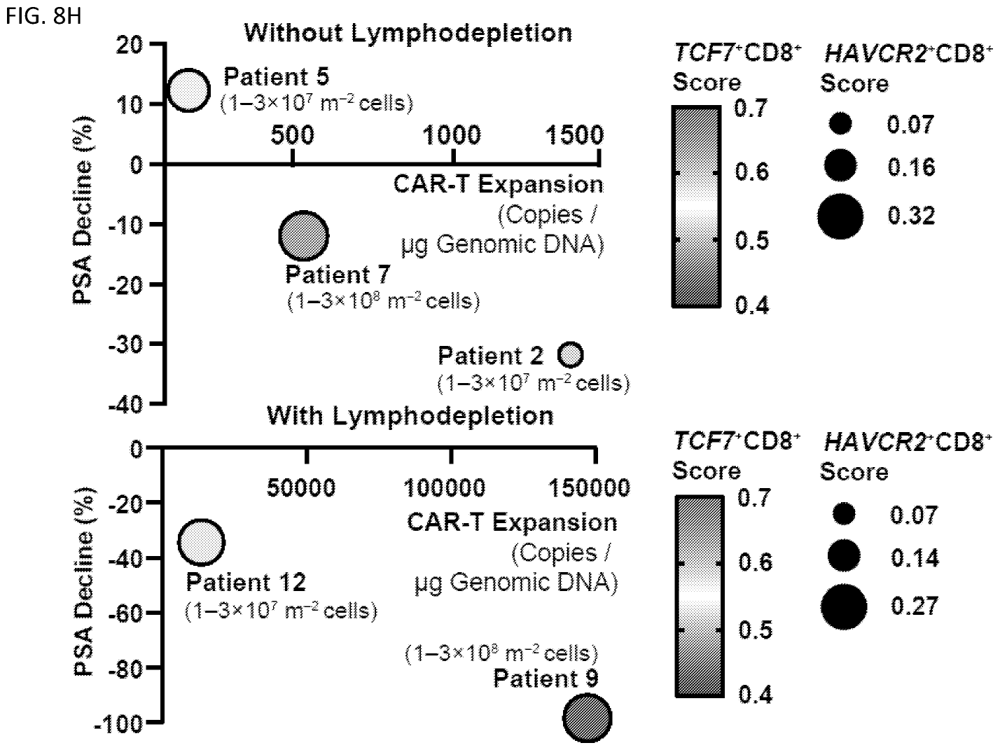

scRNA-seq was performed on five TGFβRII armored PSMA CAR T-cell infusion products administered to mCRPC cancer patients (FIG. 8A). The infusion products were selected based on sample availability and to represent a range of clinical readouts including in vivo CAR-T expansion and prostate-specific antigen (PSA) response. 20,702 cells were obtained after eliminating low-quality cells, which consisted mostly of T-cells and rare B-cells (FIG. 8B-8C). To identify transcriptional features that are associated with CAR T-cell therapeutic potency, CD8 T-cells were focused on. Clustering all CD8⁺ T cells revealed four major cell states: early memory-like TCF7⁺CD8⁺ and CCR7⁺ CD8⁺ clusters; and effector-like GZMA⁺CD8⁺ and TIM3⁺ CD8⁺ clusters (FIG. 1A, FIG. 8D).

Notably, the TCF7⁺CD8⁺ subset was the only population that expressed a high level of TCF7 (FIG. 1I, FIG. 8D). In accordance with this TCF7 expression profile, TCF7⁺CD8⁺ clusters were significantly enriched in TCF7⁺ stem cell-like T-cell signatures that are associated with robust antiviral responses in mouse models of acute and chronic lymphocytic choriomeningitis virus (LCMV) infection (FIG. 1C). In contrast, the TIM3⁺CD8⁺ cluster was markedly enriched in T-cell exhaustion signatures, whereas CCR7⁺CD8⁺ and TCF7⁺CD8⁺ populations had low exhaustion scores (FIG. 1D). Consistent with the enrichment analysis, the TIM3⁺CD8⁺ population exhibited upregulation of multiple inhibitory molecule transcripts compared to TCF7⁺CD8⁺ cells (FIG. 8E).

A recent study that characterized leukapheresed T-cells of pediatric ALL patients demonstrated that enrichment of an interferon (IFN) response signature is associated with poor CAR T-cell persistence (Yong et al. Immunology and Cell Biology 2017; 95(4):356-63). CD8⁺ T-cell clusters identified in this analysis were examined for this battery of genes. TIM3⁺CD8⁺ T-cells were found to have significantly higher IFN response signature scores compared to the other populations (FIG. 1E). The TIM3⁺CD8⁺ state is associated with short CD19 CAR T-cell persistence in ALL, which often prognosticates poor outcome (FIG. 1E).

CD8 clusters were investigated for association with response to CAR T-cells in LBCL. Intriguingly, TCF7⁺CD8⁺ population highly expressed a gene set enriched in anti-CD19 CAR T-cell infusion products of complete response (CR) patients, whereas the TIM3⁺CD8⁺ T-cell population exhibited the highest scores for a gene set that characterizes non-responder (NR) anti-CD19 CAR T-cells (FIG. 1G). These results were concordant with exhaustion signatures in independent data sets (FIG. 8F). PSMA CAR-T infusion products were scored for TCF7 and TIM3 profiles and analyzed how these signature scores correlate with PSA decline in mCRPC patients following CAR T-cell transfer (FIGS. 8G, 8H). In accordance with recently reported clinical findings, there is a clear dose- and lymphodepletion-dependent relationship with peripheral blood CAR T-cell expansion and early antitumor effects, as determined by serum PSA decrease (FIG. 8H). Patients 2 and 5 were treated in the cohort that received the lowest dose of CAR T-cells in this trial, without lymphodepleting preconditioning prior to cell infusion. It is intriguing that independently of conditioning and cell dose, Patient 2 pre-infusion CAR T-cells with a highly enriched TCF7 signature and a low TIM3 score exhibited superior in vivo expansion and PSA response induction, compared to the infusion product of Patient 5 with lower TCF7 and elevated TIM3 population scores (FIGS. 8G, 8H). These results suggest that increasing TCF7CD8⁺ populations and depleting TIM3⁺CD8⁺ populations in CAR T-cell infusion products may enhance CAR T-cell efficacy.

Among the transcription factors upregulated in the TIM3⁺CD8⁺ population, the study focused on BLIMP1 (encoded by PRDM1 (FIGS. 1I-1J). PRDM1 is known to play a central role in driving T-cell exhaustion and terminal differentiation. In addition, high expression of PRDM1 is associated with loss of T-cell sternness and self-renewal capacity through reciprocal repression of TCF7. Consistent with these results, TIM3⁺CD8⁺ cells displayed the highest expression level of PRDM1, whereas TCF7⁺CD8⁺ T-cells exhibited low levels of PRDM1 expression among the CD8⁺ clusters (FIG. 1H). To further validate these findings, PRDM1 and TCF7 expression were compared in cohorts of chronic lymphocytic leukemia (CLL) patients treated with autologous CD19 CAR T-cells. Pre-infusion CAR T-cells from CR and very good partially responding (PR_{TD}) Subjects exhibited significantly lower levels of PRDM1 and elevated TCF7 expression, compared to poorly functional products from non-responders (NR) or conventional partial responders (PR) (FIG. 1G).

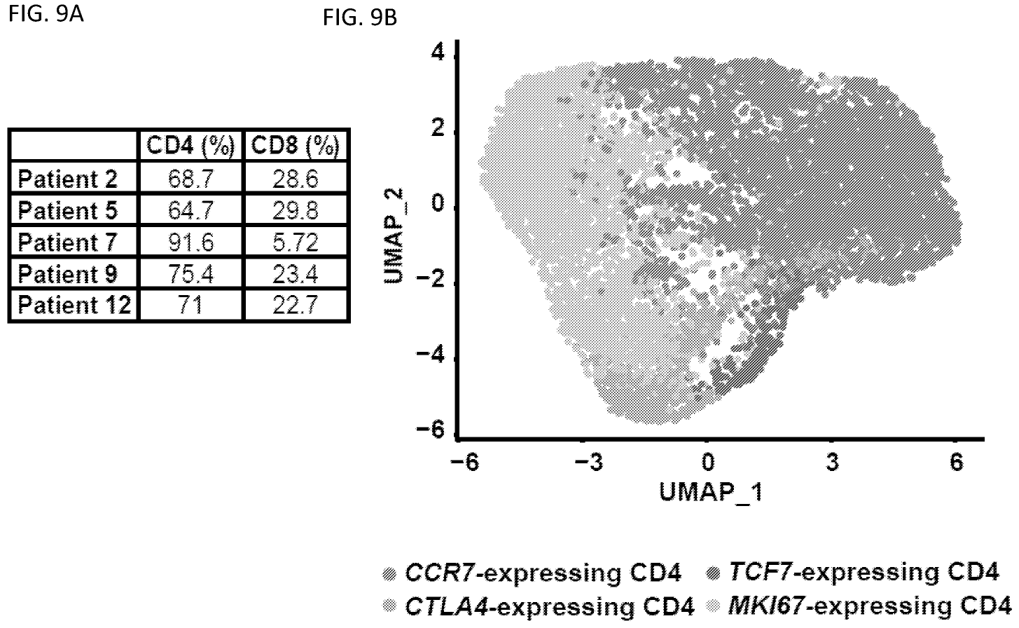

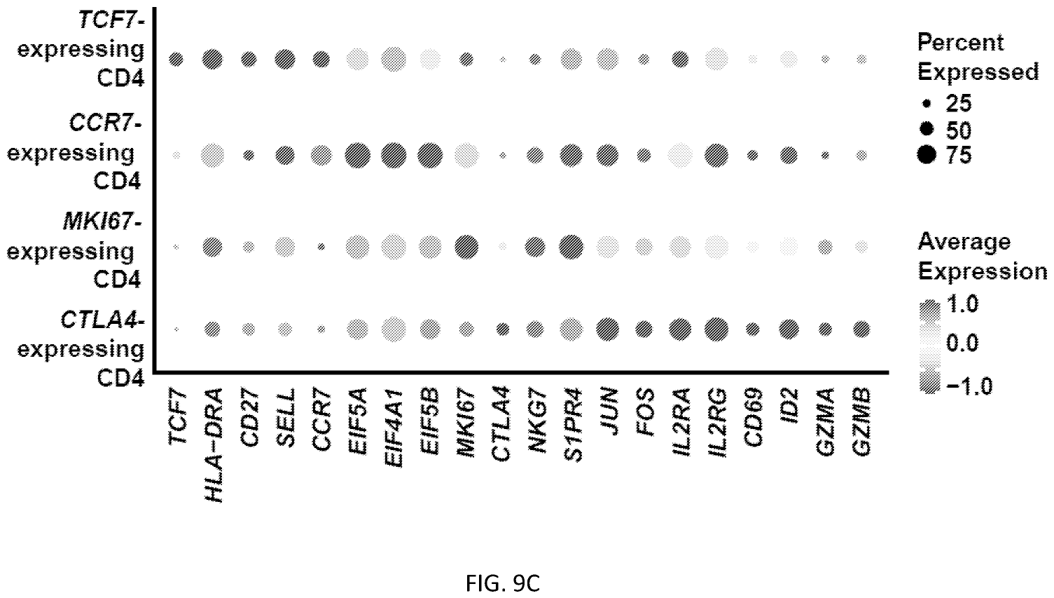

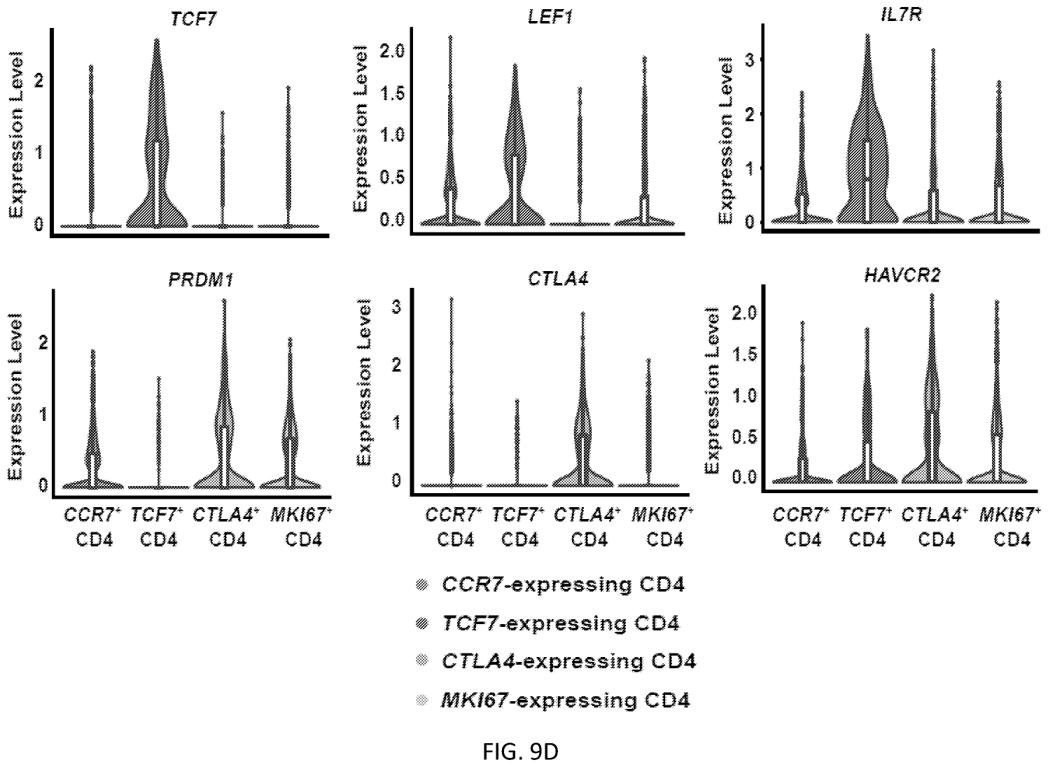

The frequencies of CD4⁺ and CD8⁺ T-cells within the clinical CAR infusion products profiled in this study are presented in FIG. 9A. scRNA-seq analysis of the CD4⁺ compartment is shown in FIG. 9B-9D. Clustering all CD4⁺ T-cells revealed four major cell states: TCF7⁺CD4⁺, CCR7⁺CD4⁺, MK167⁺CD4⁺, and CTLA4⁺CD4⁺ clusters. CTLA4⁺CD4⁺ cells displayed the highest expression level of PRDM1 whereas TCF7⁺CD4⁺ T-cells exhibited low levels of PRDM1 expression among the CD4⁺ clusters (FIG. 9D). It was originally hypothesized that PRDM1 mediates CAR T-cell exhaustion and attrition of sternness. Thus, PRDM1 KO may mitigate T-cell exhaustion and improve CAR T-cell expansion, persistence and antitumor efficacy.

### Example 2: CRISPR/Cas9-Mediated PRDM1 KO Improves Early Memory Differentiation of PSMA CAR T-Cells

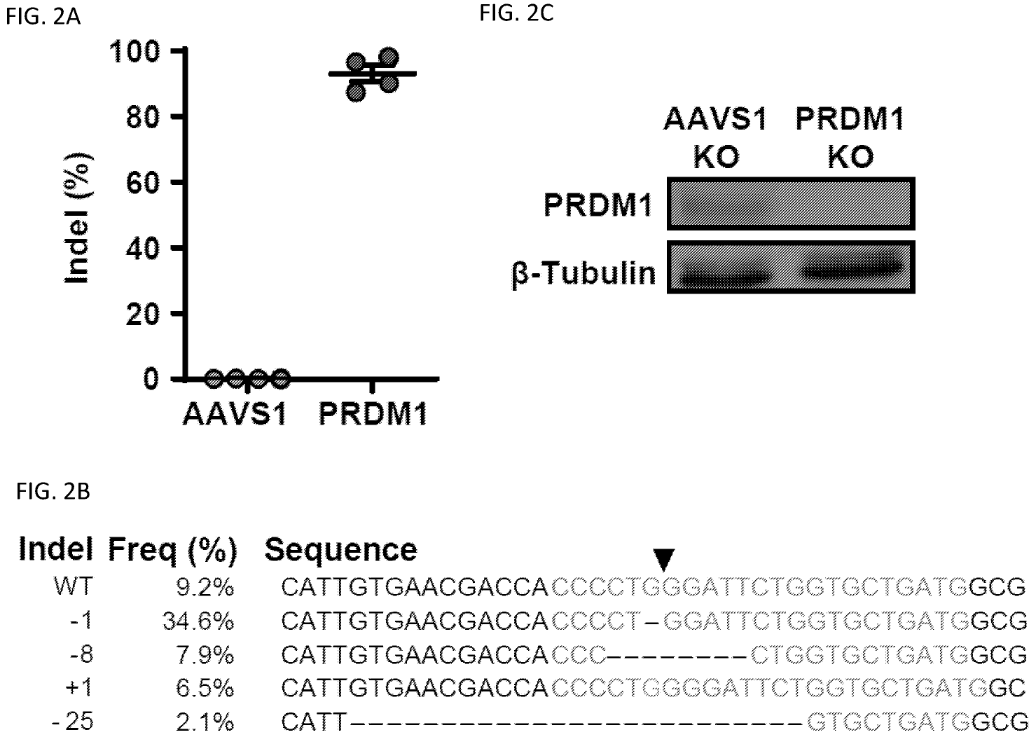

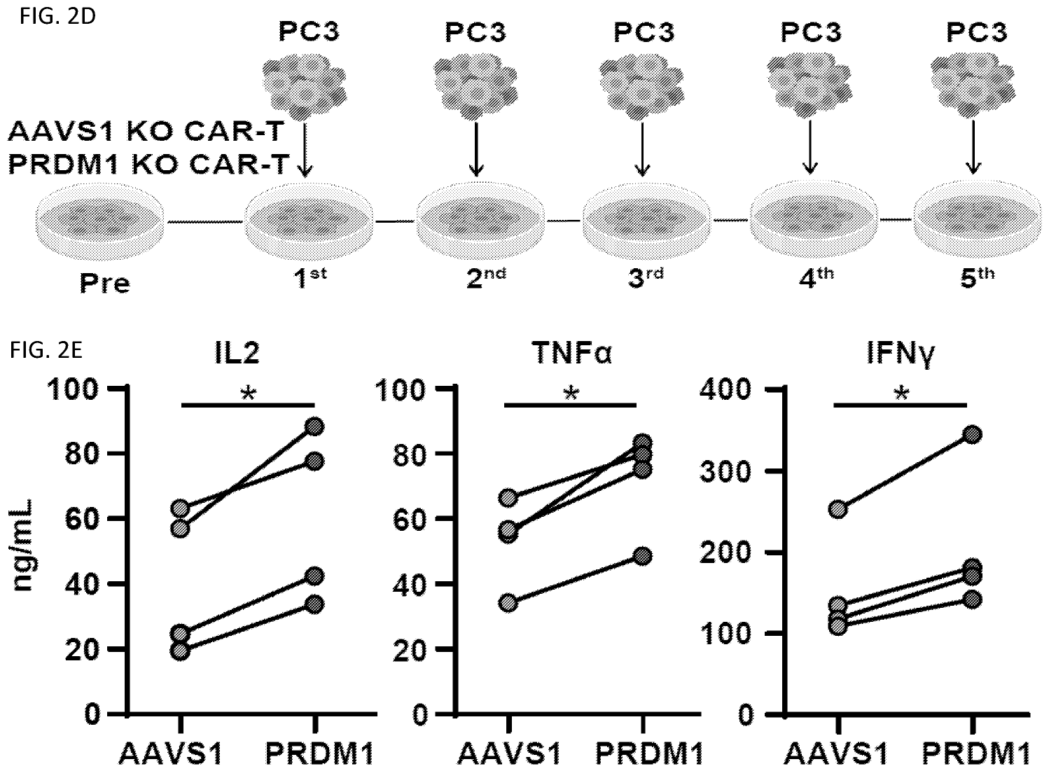

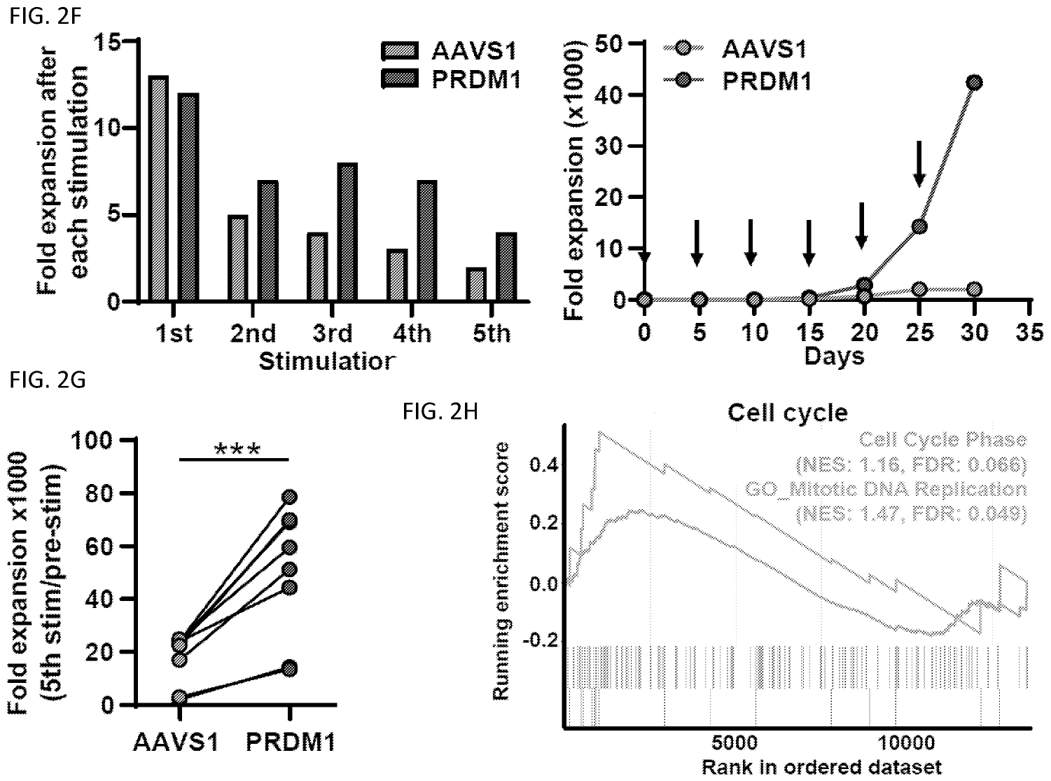

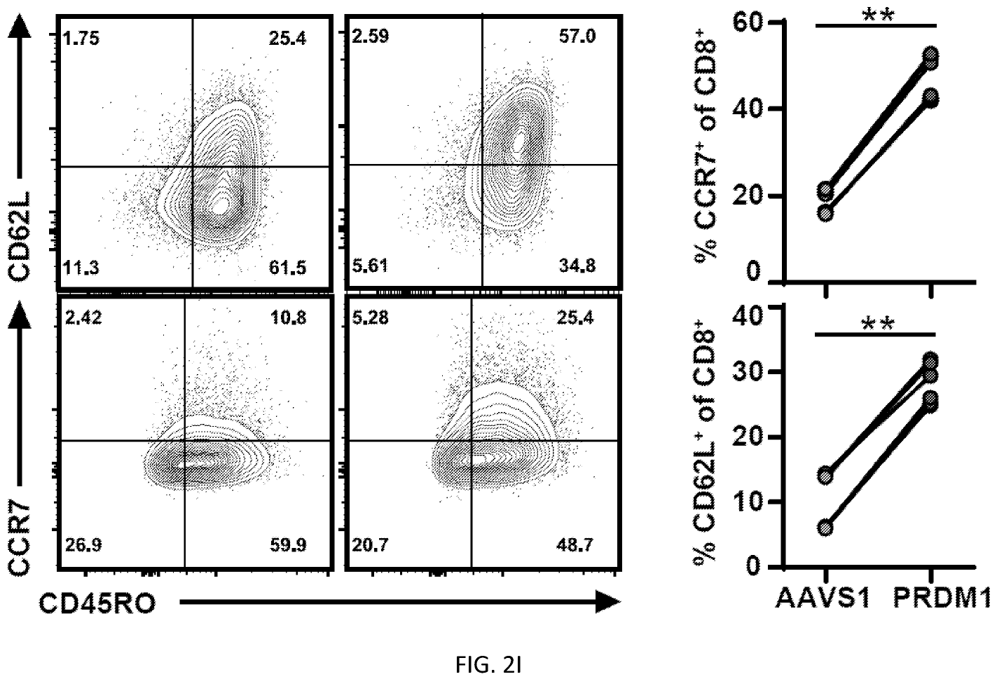

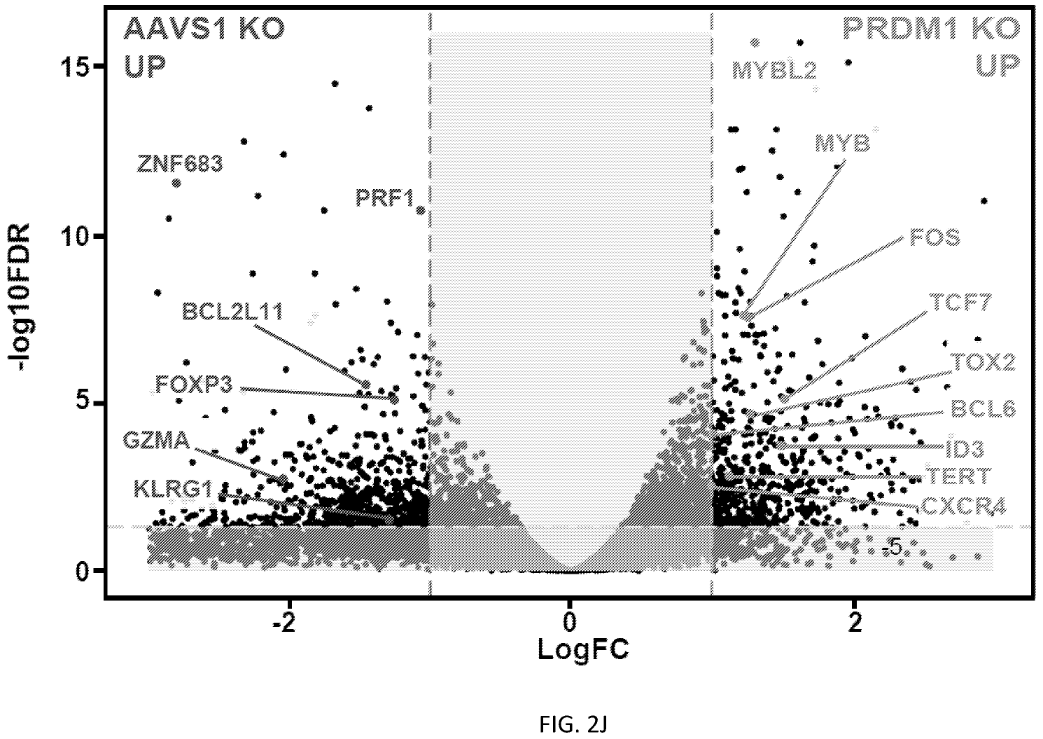

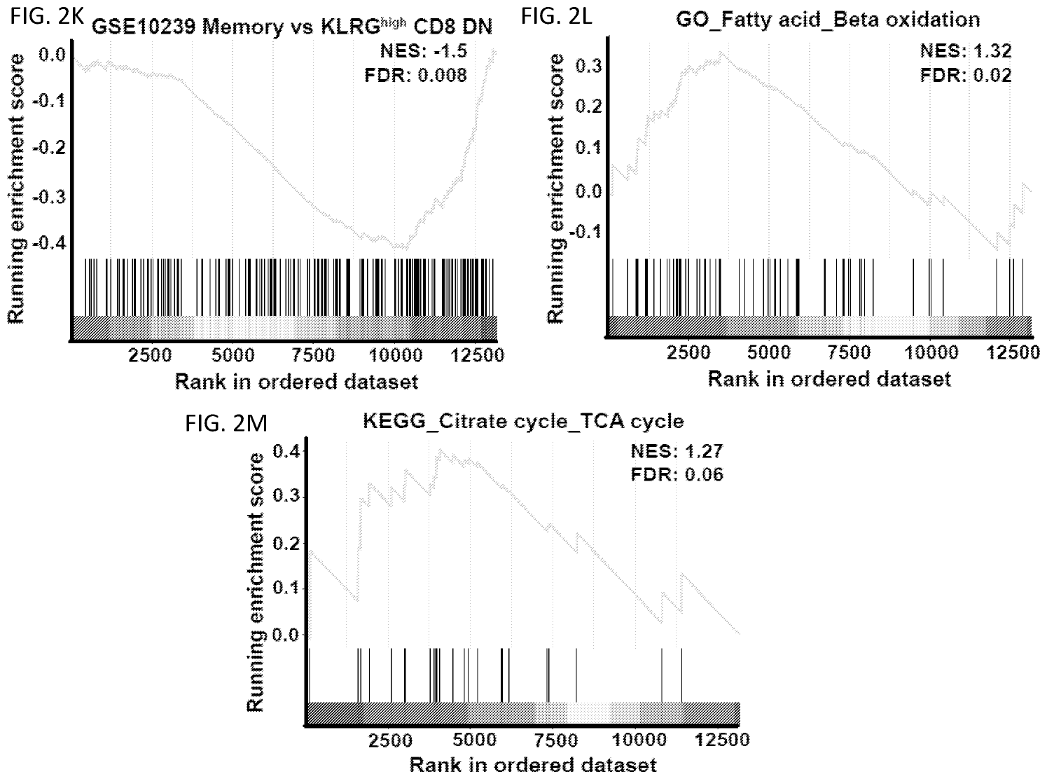

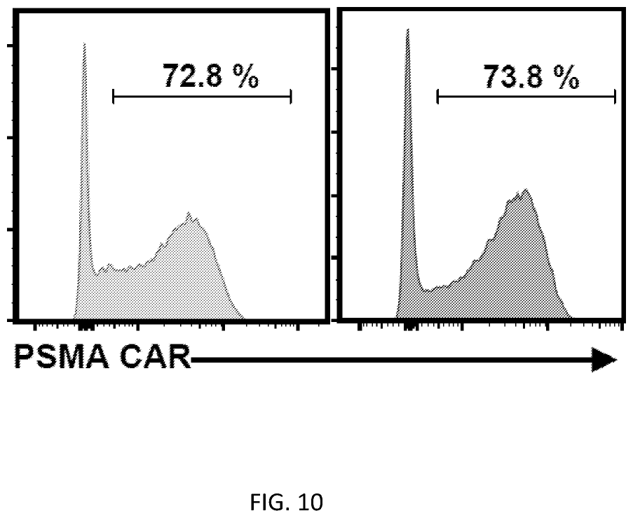

To examine how BLIMP1 deficiency affects CAR T-cell fate, PRDM1 was knocked out in human primary T-cells using CRISPR/Cas9 (FIGS. 2A-2C). CAR T-cells with gene editing at safe harbor locus, AAVS1, were used as a control in in vitro and in vivo studies. PSMA CAR expression levels were comparable between control AAVS1 KO and PRDM1 KO CAR T-cells (FIG. 10). Next, the phenotype of PRDM1 KO CAR T-cells was characterized during an in vitro ‘stress test’ involving repetitive antigen stimulation (FIG. 2D). PRDM1 KO CAR T-cells showed increased effector cytokine production compared to control CAR T-cells 24-hours after the first restimulation (FIG. 2E). Also, while expansion capacity of AAVS1 KO CAR T-cells gradually declined upon repetitive stimulation, PRDM1 KO CAR T-cells sustained high proliferative capacity even after multiple rounds of restimulation and concomitantly upregulated cell cycle-related gene signatures (FIGS. 2F-2H). This enhanced expansion capacity of PRDM1 KO CAR T-cells was attributed to increased memory formation. PRDM1 KO CAR T-cells increased expression of CD62L, CCR7, MYB, ID3, and TCF7 and enriched transcriptomic signatures of memory precursor effector cells, fatty acid oxidation, and tricarboxylic acid cycle, indicating that PRDM1 KO CAR T-cells are skewed toward early memory fate (FIGS. 2I-2M).

### Example 3: PRDM1 KO Increases TCF7 Signatures and Enhances Early Memory in a TCF7-Dependent Manner

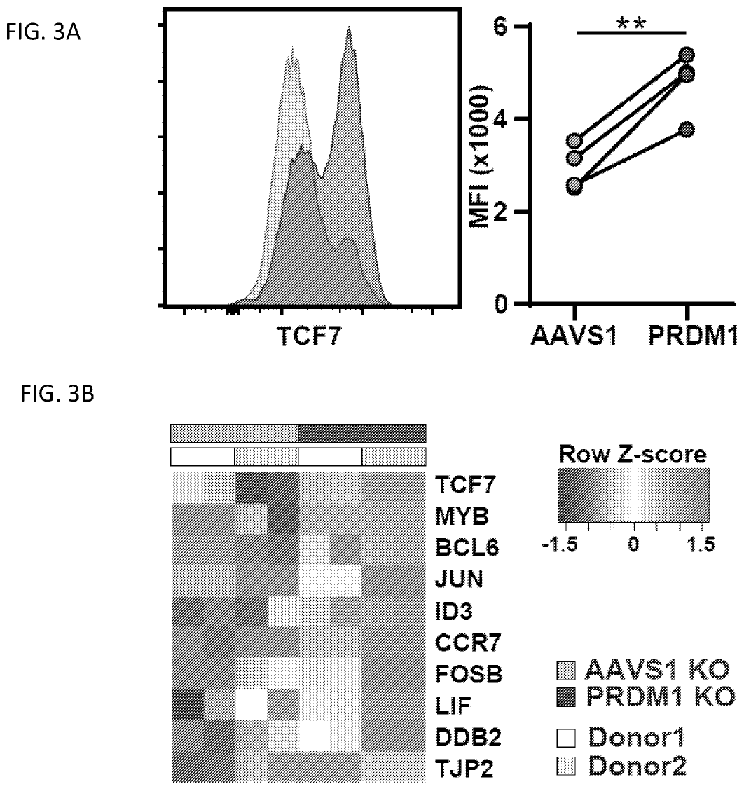

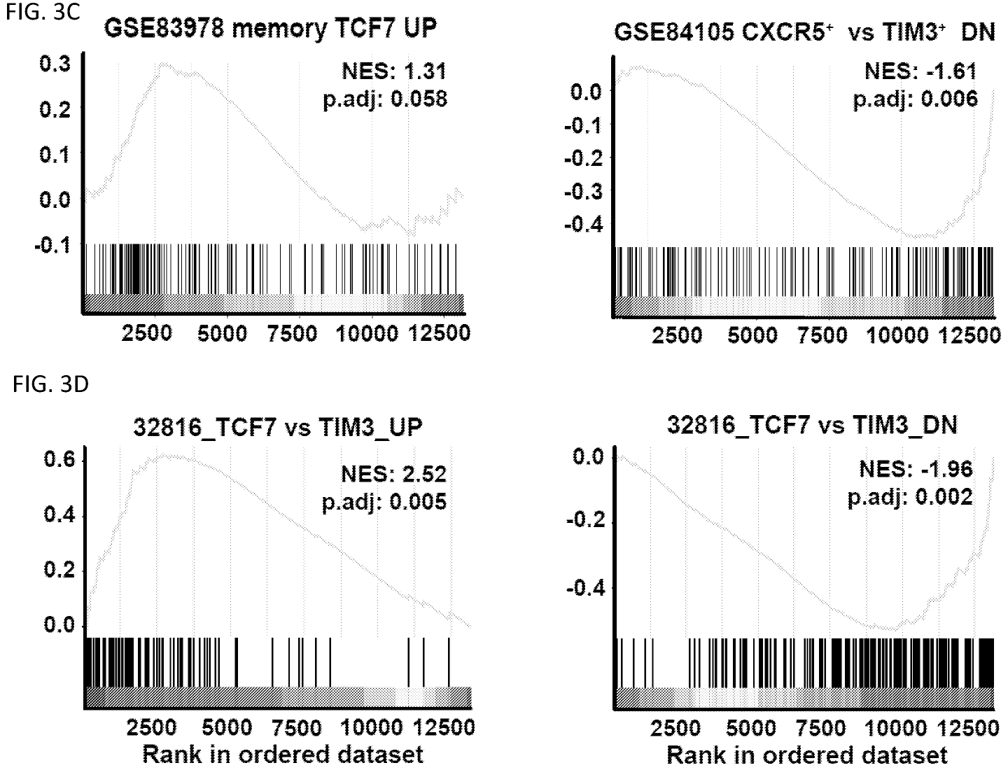

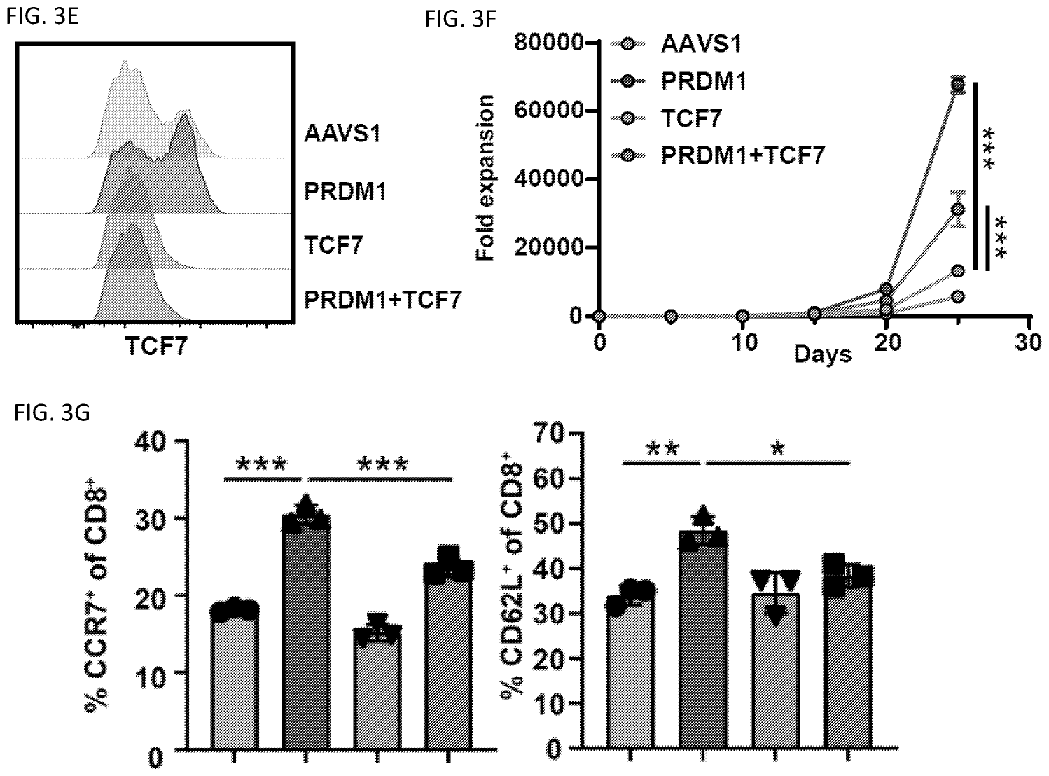

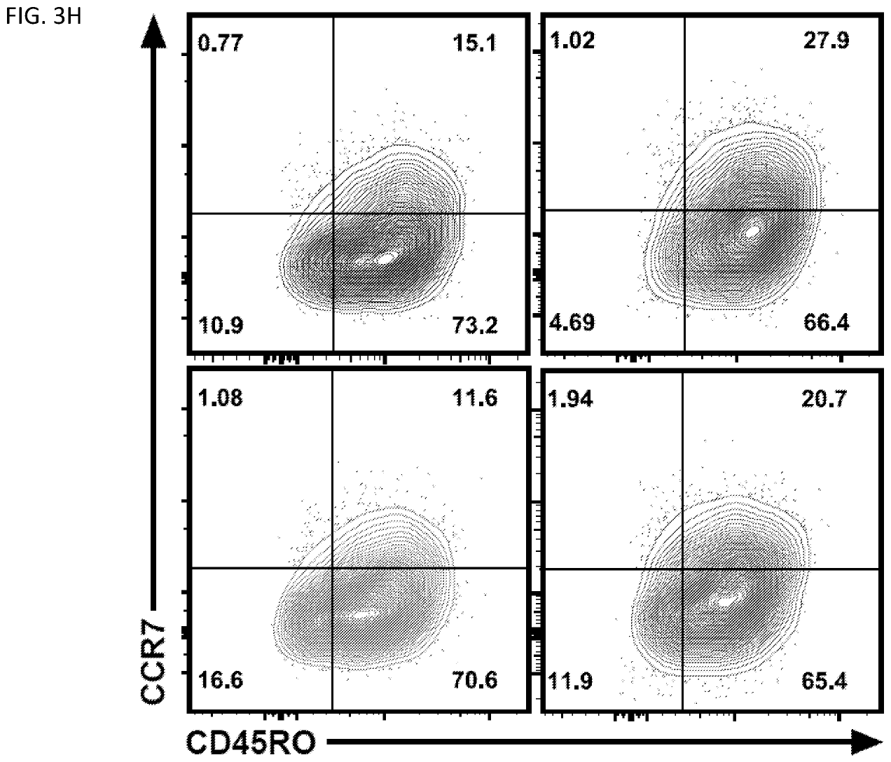

Next, it was investigated whether PRDM1 KO derepresses TCF7 expression. PRDM1 KO increased expression of TCF7 and genes encoding other transcription factors that are crucial for maintaining T-cell stemness, such as MYB, BCL6 and ID3 (FIGS. 3A-3B). Moreover, PRDM1 KO CAR T-cells were significantly enriched TCF7T stem cell-like T-cell signatures, suggesting that PRDM1 KO inhibits TCF7-mediated stemness (FIG. 3C). Notably, PRDM1 KO CAR T-cells exhibited depletion of TIM3⁺CD8⁺ gene signatures and transcriptionally resembled TCF7⁺CD8⁺ T-cells observed in PSMA CAR T-cell infusion products (FIG. 3D).

To examine whether TCF7 upregulation is required for maintenance of early memory differentiation and robust CAR T-cell proliferative potential as observed in FIGS. 2A-2M, TCF7 was knocked out and CAR T-cell expansion was assessed in a restimulation assay (FIG. 3E). TCF7 depletion significantly counteracted the effect of PRDM1 KO by reducing the proliferative capacity and CCR7 and CD62L expression (FIGS. 3F-3H). Lack of polyfunctionality is a hallmark of terminal differentiation and T-cell dysfunction. PRDM1 and TCF7 single or double knockout CAR T-cells were stimulated and expression of IL2, IFNγ and TNFα was measured to assess polyfunctionality. PRDM1 ablation increased polyfunctional CAR T-cells compared to AAVS1 control CAR T-cells (FIG. 31), which coincided with increased early memory of PRDM1 KO CAR T-cells as observed previously. Conversely, TCF7 depletion decreased the frequency of the polyfunctional PRDM1 KO CAR-T populations (FIG. 31), suggesting that PRDM1 enhanced CAR-T polyfunctionality in part via TCF7 upregulation. Taken together, these data imply that PRDM1 knockout enhances early memory of CAR T-cells in a TCF7-dependent manner.

### Example 4: PRDM1 KO Hampers T-Cell Effector Function and Tumor Control During Chronic CAR Stimulation Despite Significant Increases in CAR T-Cell Proliferative Capacity

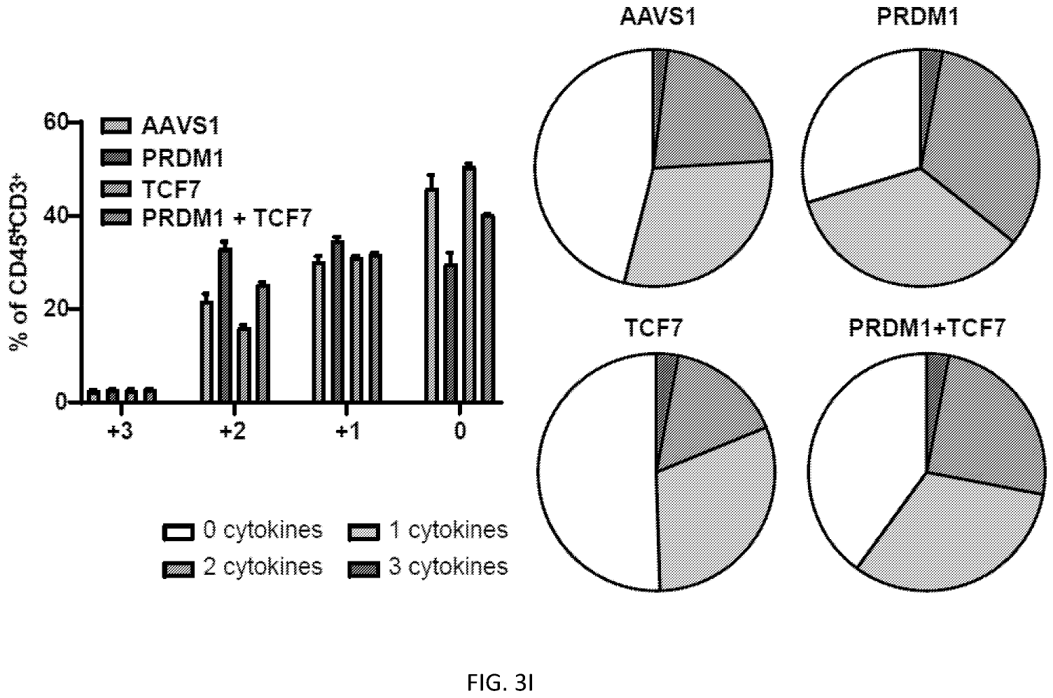

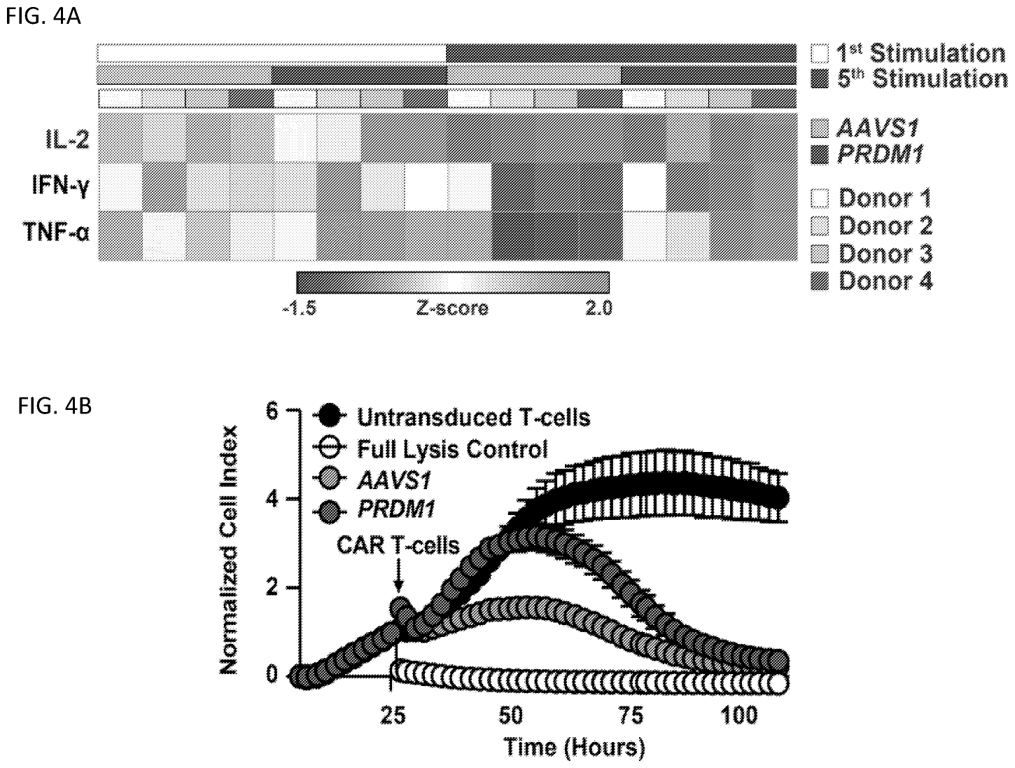

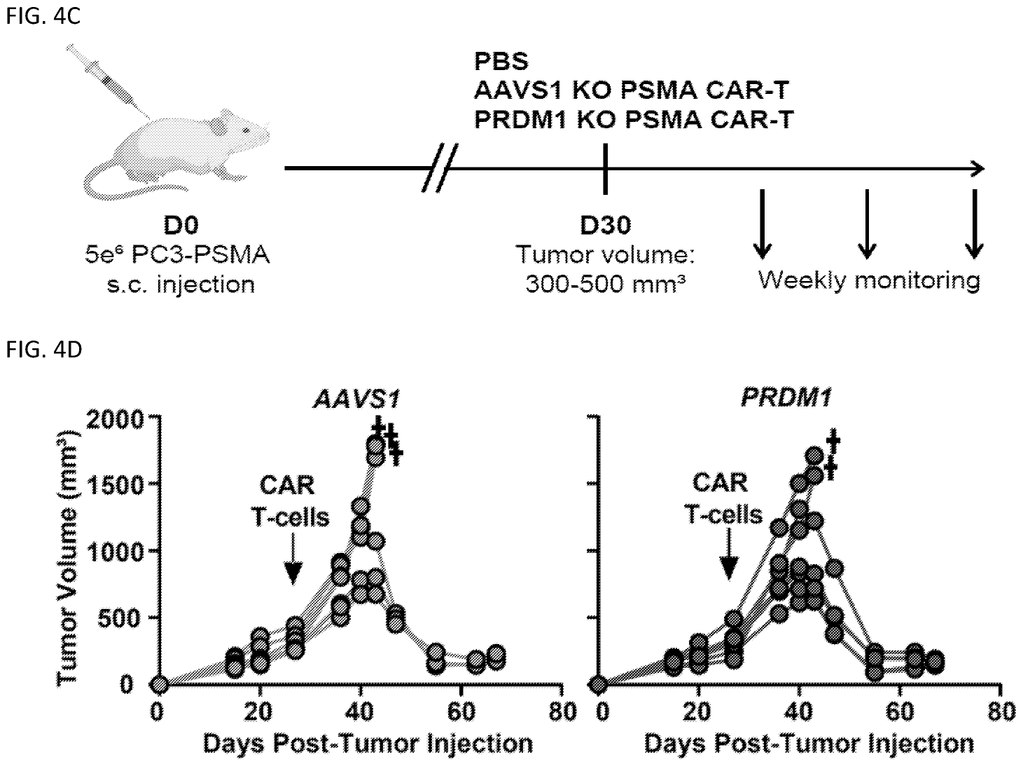

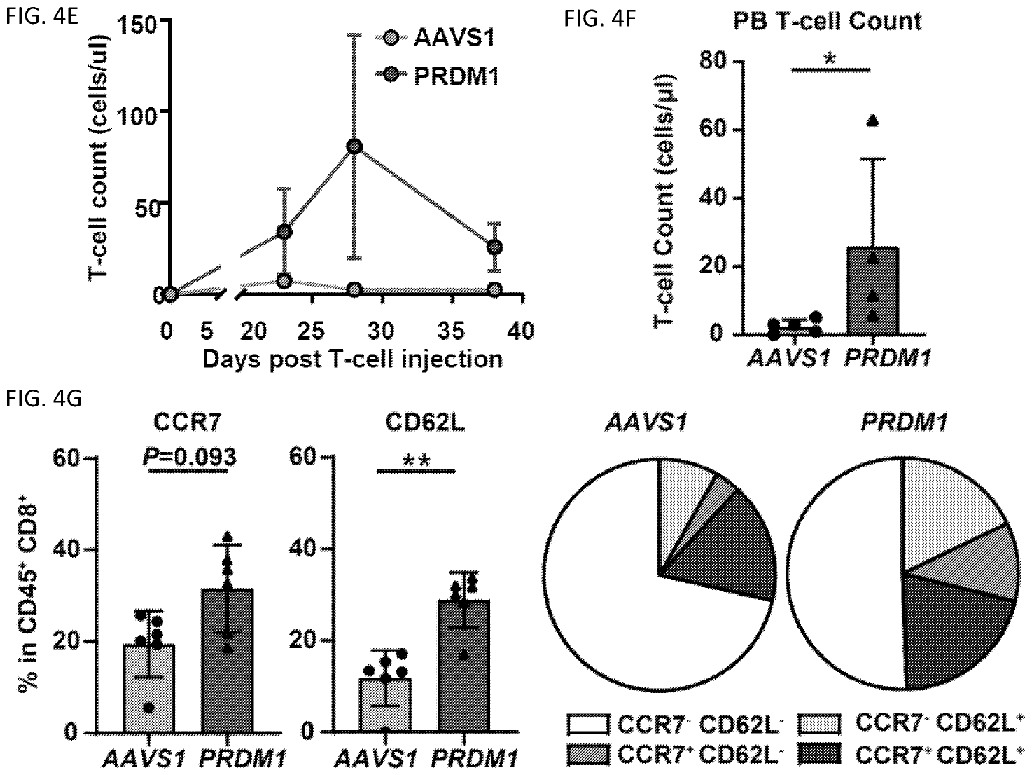

To evaluate the effector functions of PRDM1 KO CAR T-cells in the setting of repetitive antigen exposure, cytokine levels were assessed 24-hours after the first and fifth rounds of in vitro PC3-PSMA tumor cell stimulation. PRDM1 KO initially increased effector cytokine production as shown in FIG. 31 and FIG. 4A. However, after multiple tumor antigen challenges, PRDM1 KO CAR T-cells exhibited dramatically reduced effector cytokine secretion (FIG. 4A). Further, after five consecutive tumor challenges, the cytolytic activity of PRDM1 KO CAR T-cells was impaired compared to that of control CAR T-cells (FIG. 4B). This result is consistent with a previous study in which PRDM1 deficiency profoundly compromised the cytotoxic activity of antigen-specific CD8⁺ T-cells during chronic viral infection.

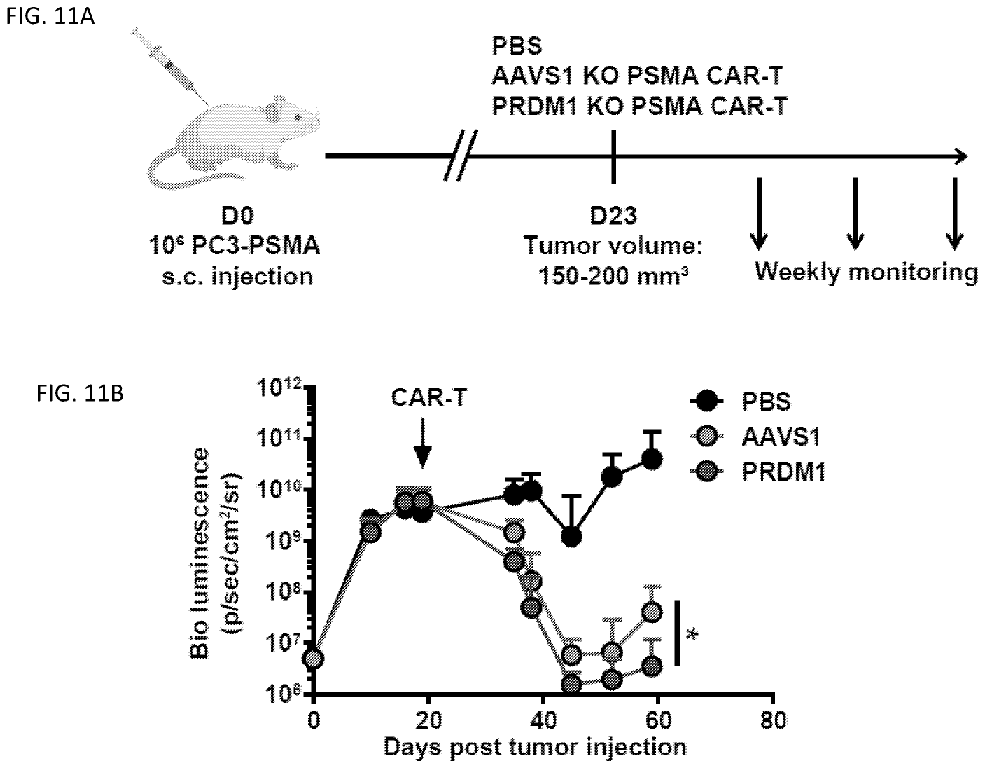

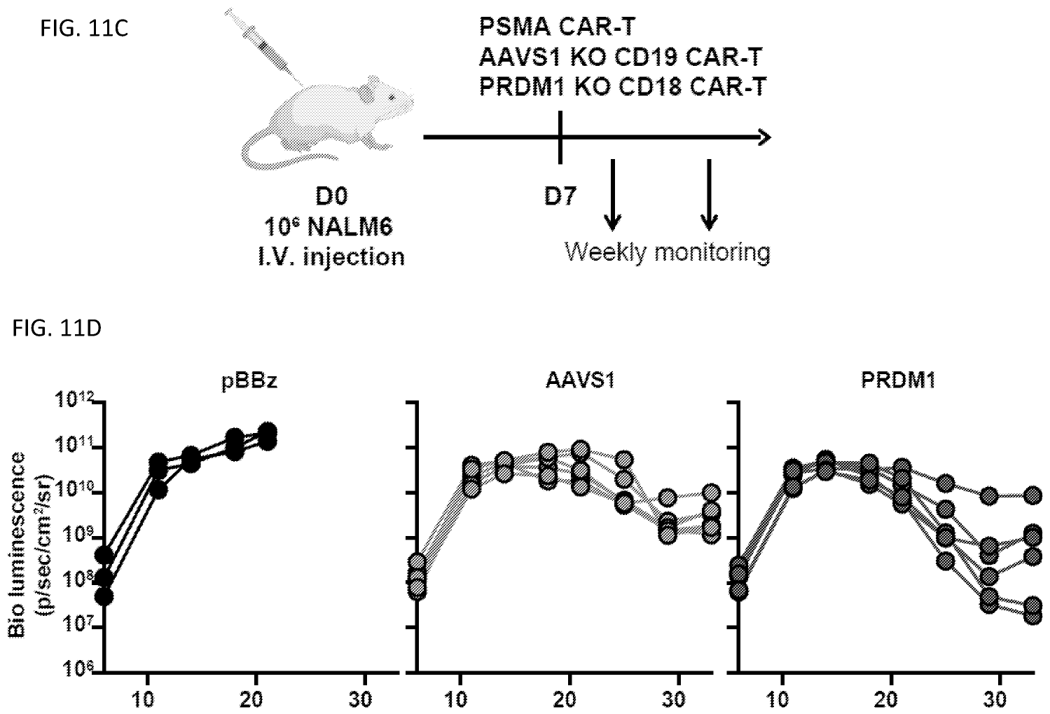

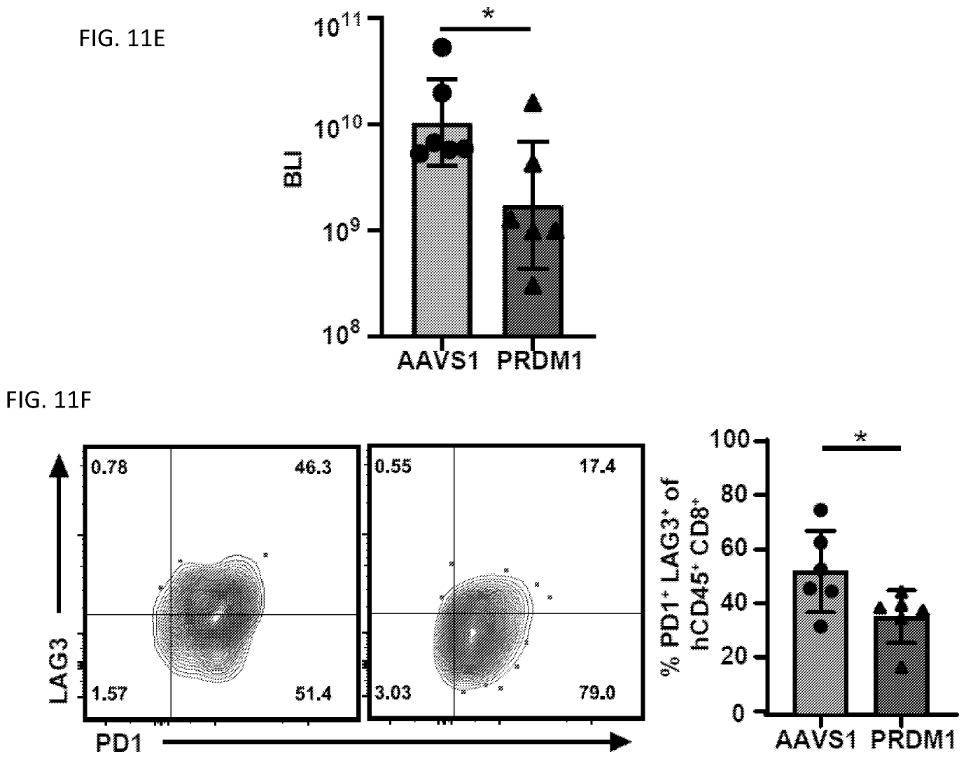

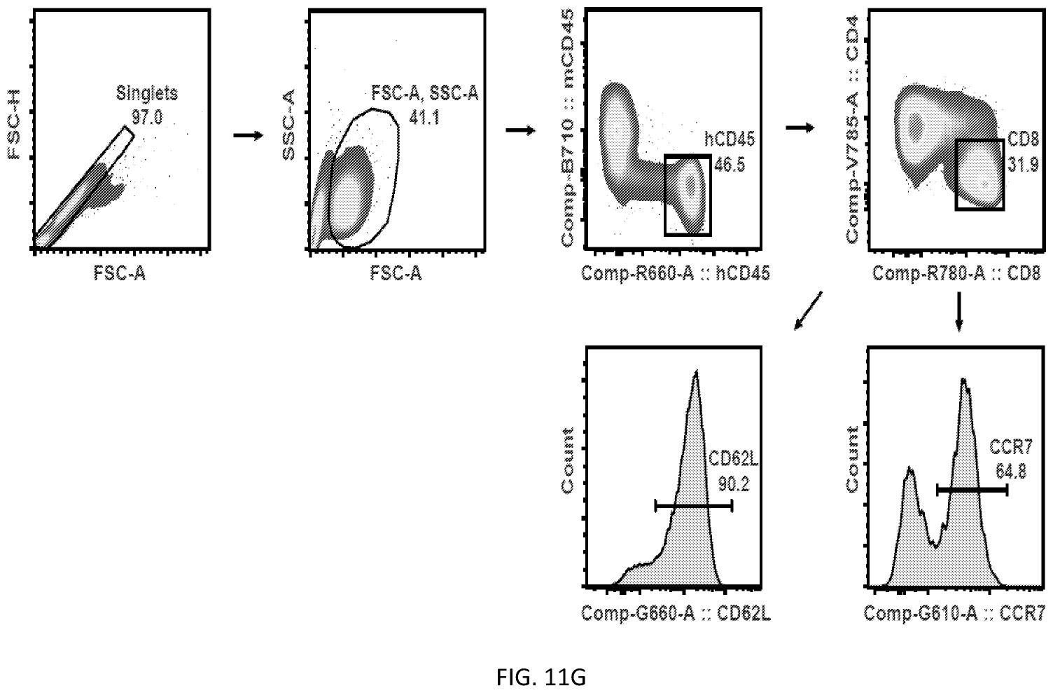

Based on the above results, the in vivo antitumor activity of PRDM1 KO CAR T-cells was assessed in xenogeneic mouse models. When tested against a relatively low burden of flank-engrafted PC3-PSMA prostate tumor cells, PRDM1 KO PSMA CAR T-cells exhibited a modest enhancement of tumor control compared to control CAR T-cells (FIGS. 11A, 11B). PRDM1 KO anti-CD19 CAR T-cells better suppressed cancer growth compared to control CAR T-cells in a B-cell acute lymphoblastic leukemia (ALL) model (NALM-6), although these CAR T-cells eventually failed to eradicate tumors (FIG. 11C-11F). In an in vivo ‘stress test’ in which tumor burden is escalated to reveal CAR T-cell functional limits, PRDM1 KO CAR T-cells showed comparable antitumor activity compared to AAVS1 KO CAR T-cells (FIGS. 4C, 4D). Despite lack of improved tumor control over AAVS1 KO CAR T cells, PRDM1 KO CAR T-cells exhibited enhanced in vivo expansion and persistence (FIGS. 4E, 4F). Additionally, consistent with in vitro studies, PRDM1 KO CAR T-cells maintained a higher fraction of central memory T-cells (FIG. 11G, FIG. 4G), indicating that PRDM1 deficiency improves CAR T-cell expansion and persistence by preserving early memory pools. Together, these results suggest that despite significant improvements in expansion and persistence, PRDM1 KO alone is not sufficient to potentiate robust and sustained CAR T-cell antitumor efficacy in aggressive tumor models.

### Example 5: PRDM1 KO CAR T-Cells Fail to Maintain High Effector Function Due to Upregulation of Exhaustion-Related Transcription Factors (TFs)

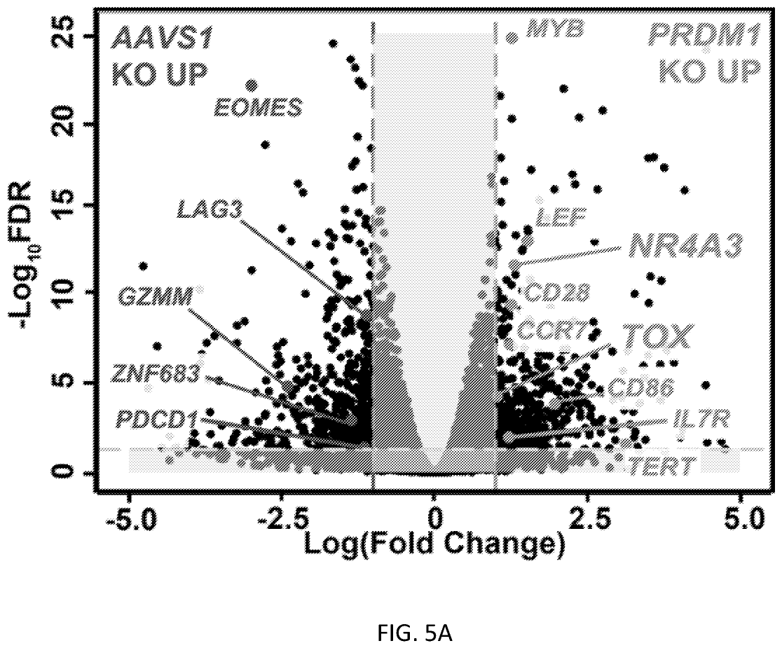

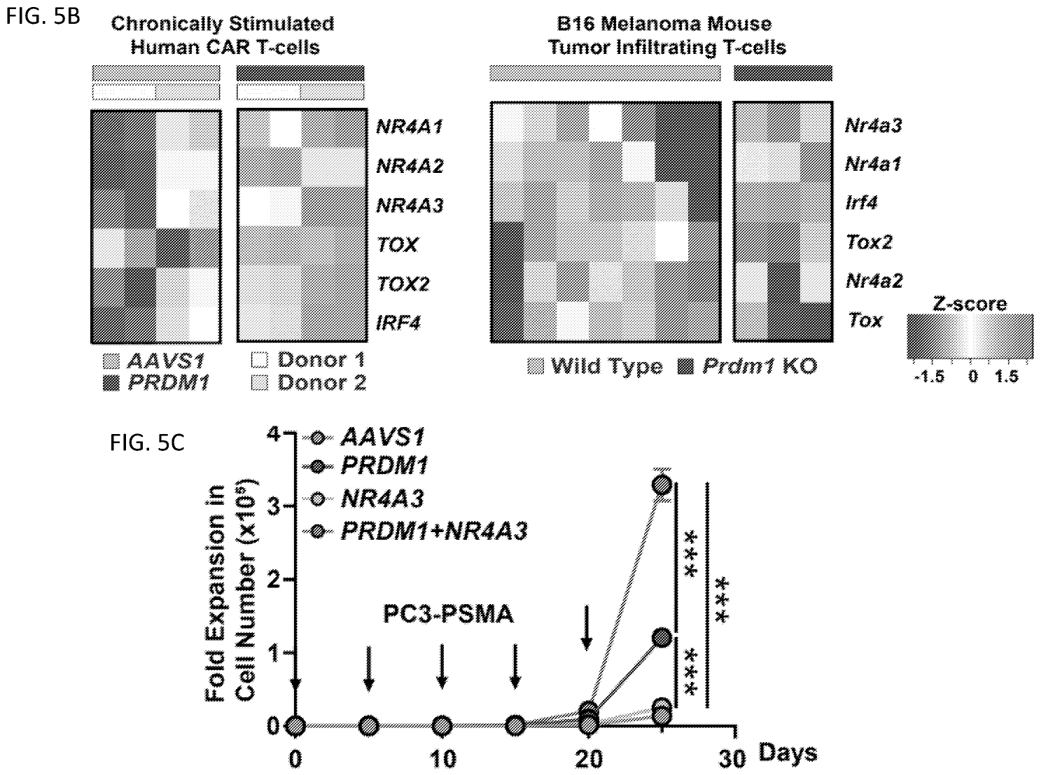

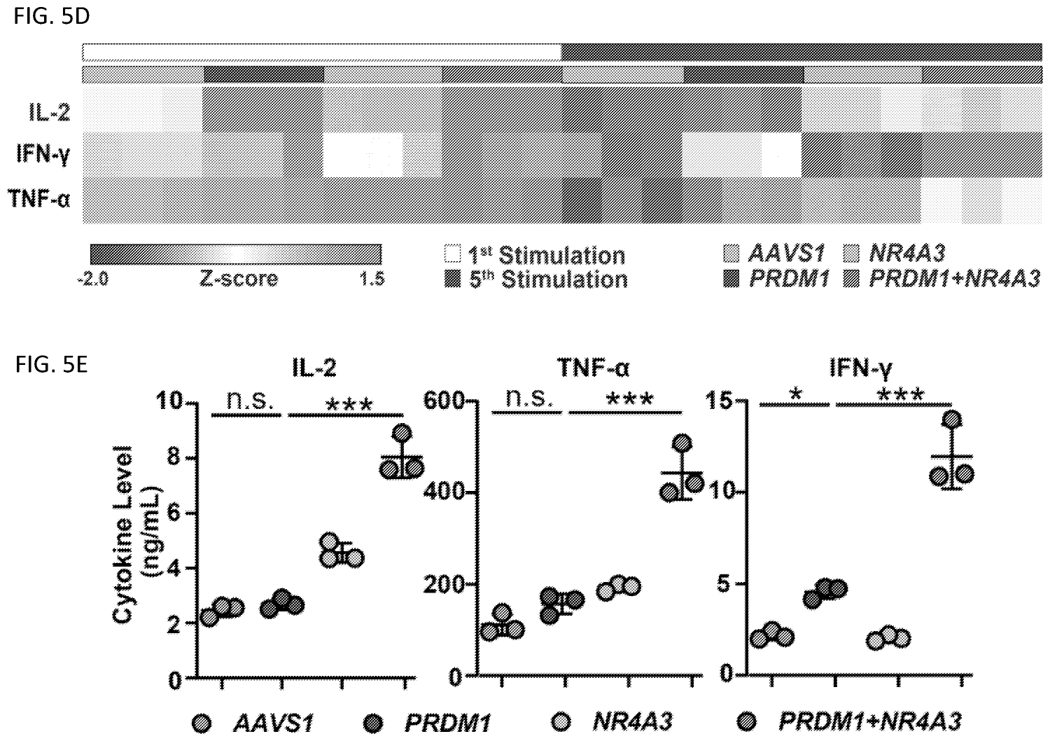

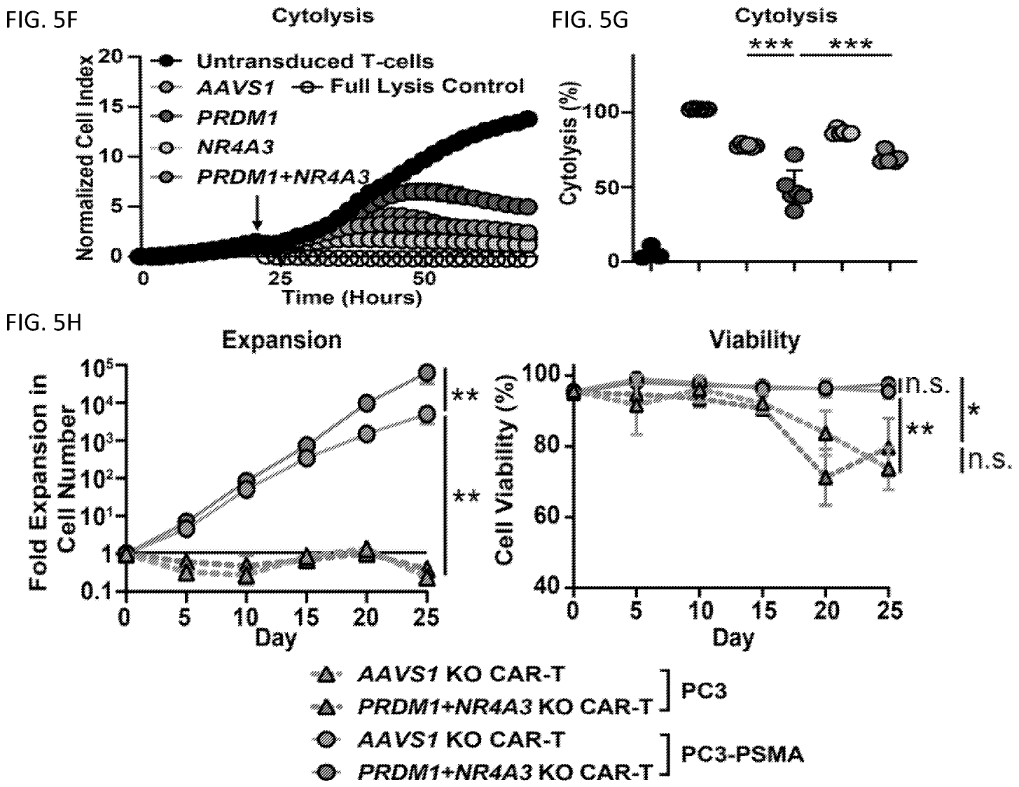

To elucidate the mechanism by which PRDM1 KO CAR T-cells fail to maintain effector function following chronic antigen stimulation, bulk RNA-seq was performed on CAR T-cells harvested after several rounds of tumor challenge. PRDM1 KO increased the expression of early memory-related genes, including MYB, LEF, CCR7, IL7R, and CD28, even after multiple stimulations (FIG. 5A). Intriguingly, together with these early memory-related genes, PRDM1 KO resulted in upregulation of genes encoding multiple exhaustion-related TFs such as the NR4A family of TFs, TOX, TOX2, and IRF4 (FIG. 5B, left). To rule out potential model-dependent effects, a publicly available RNA sequencing expression dataset of CD8⁺ TILs from B16F10 (melanoma) tumor-bearing PRDM1 conditional knockout (cKO) syngeneic mice was used. Concordant with the findings in CAR T-cells, PRDM1 cKO CD8⁺ TILs exhibited upregulated expression of NR4A3, NR4A1, and IRF4 compared to wild type counterparts (FIG. 5B, right).

### Example 6: Combinatorial PRDM1 and NR4A3 KO Sustains the Effector Function of Chronically-Stimulated CAR T-Cells

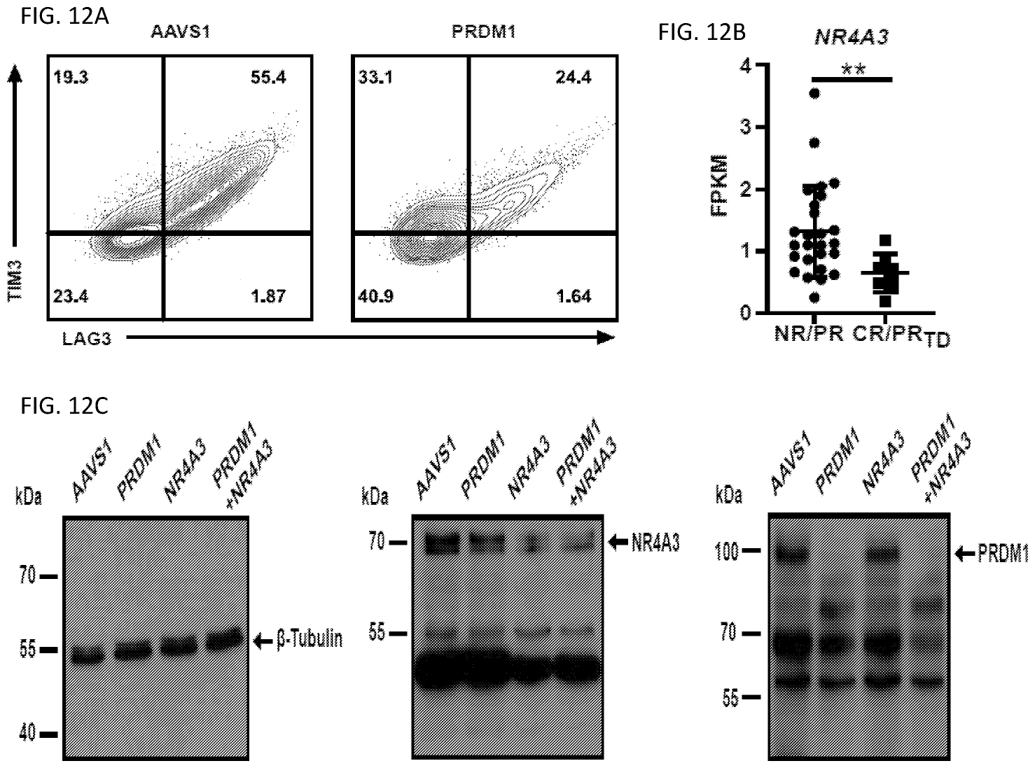

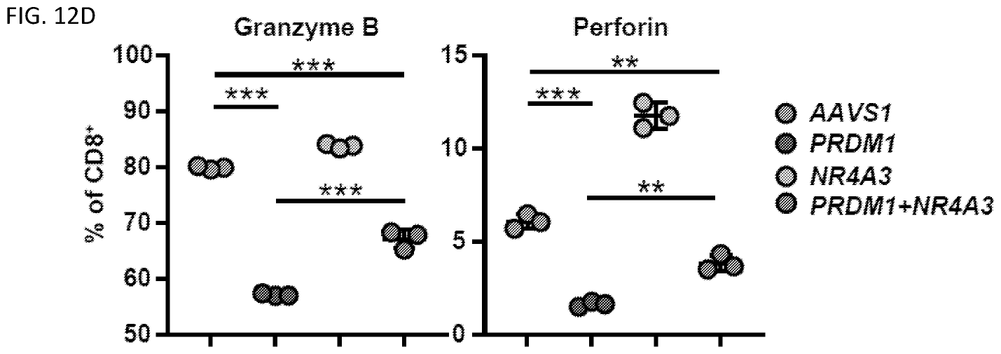

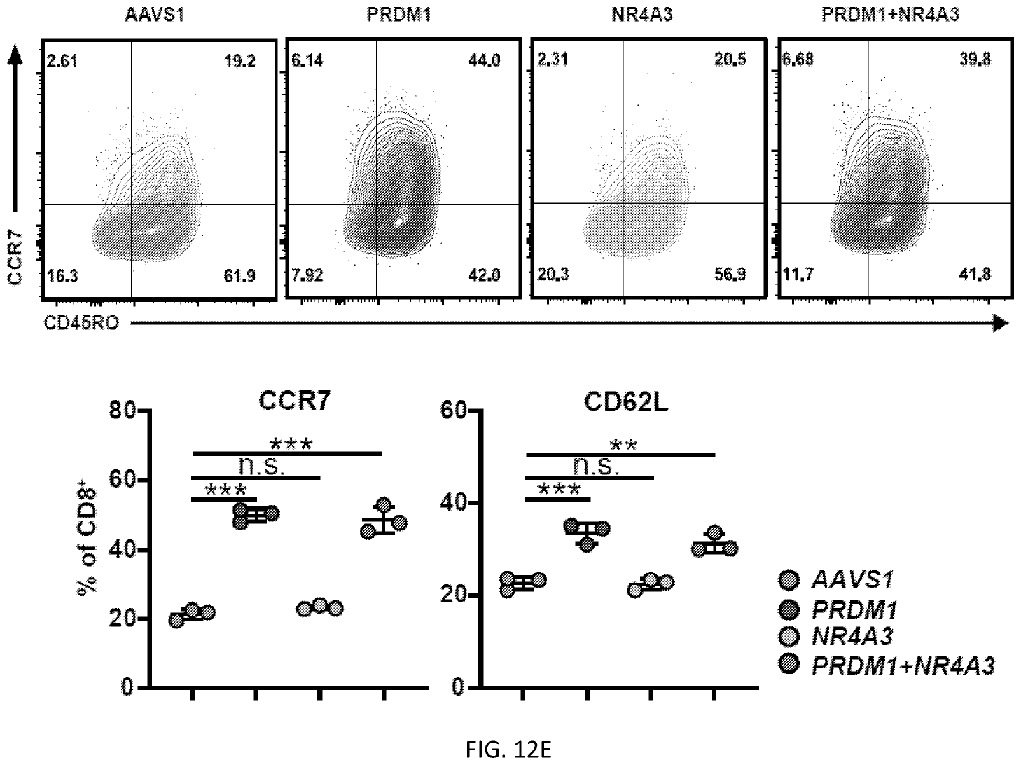

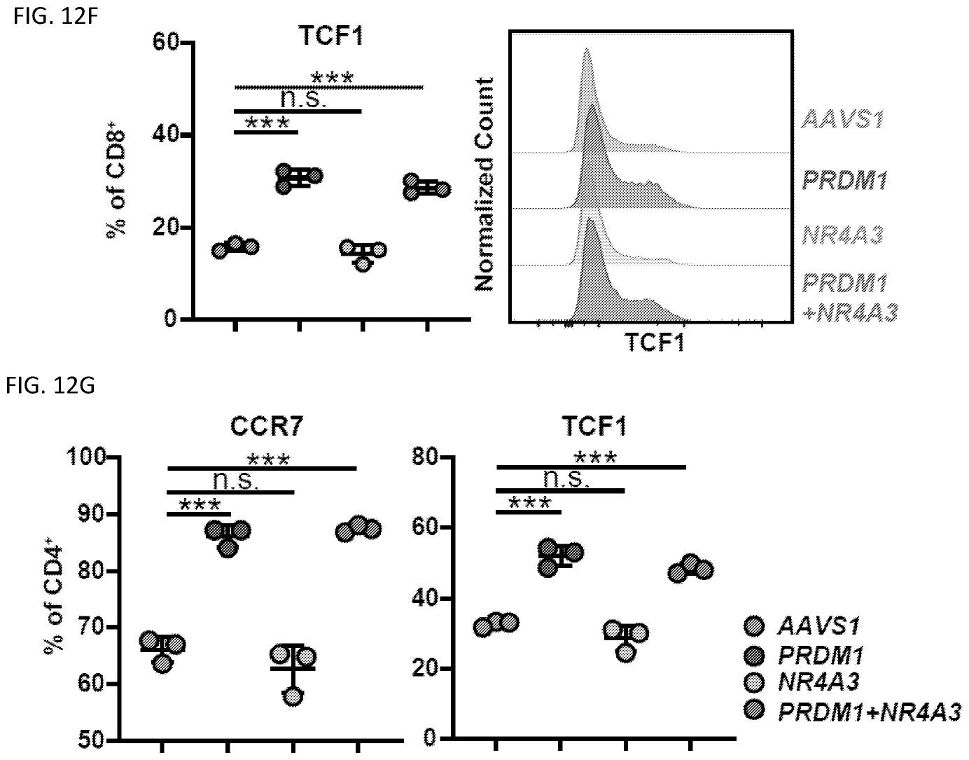

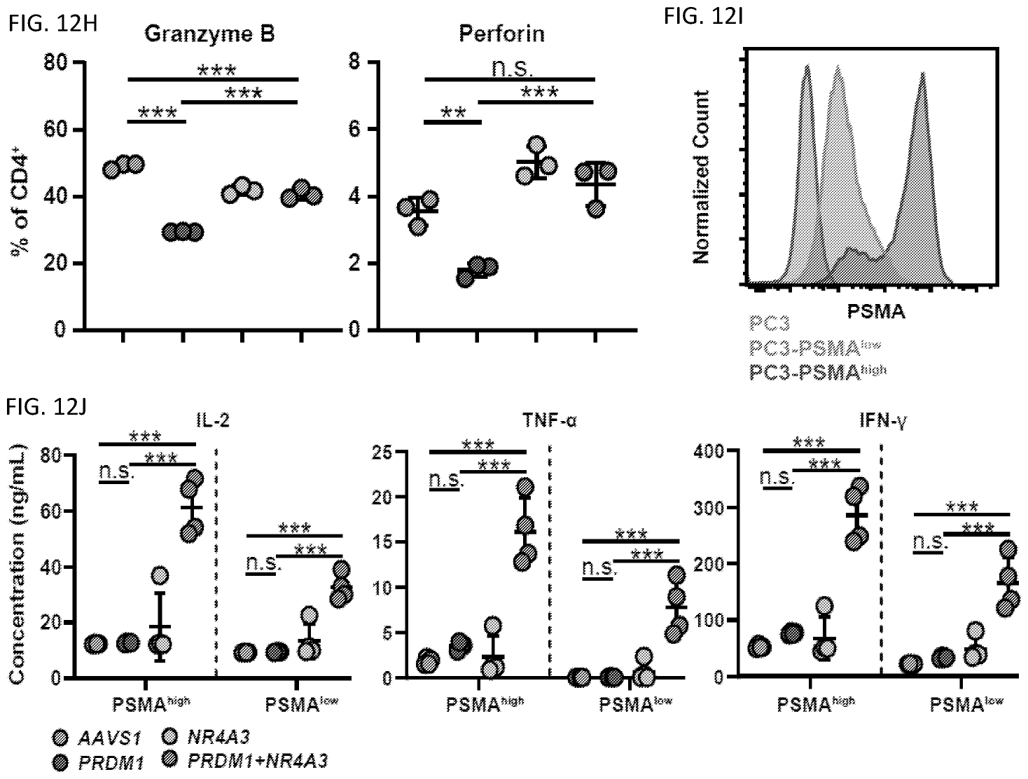

It was hypothesized that the observed compensatory upregulation of exhaustion-associated TF genes can limit the effector function of PRDM1 KO CAR T-cells during chronic stimulation and that deletion of these exhaustion factors will render PRDM1 KO CAR T-cells capable of maintaining antitumor effector function. Interestingly, PRDM1 KO CAR T-cells exhibited dramatically reduced effector function, despite inhibitory receptor downregulation (FIG. 12A). For example, PRDM1 KO CAR T-cells dramatically reduced cytokine production to a similar degree as control CAR T-cells during chronic stimulation (FIG. 4A), indicating that regulation of inhibitory receptor expression is decoupled from effector cytokine elaboration. Because NR4A3 was the most significantly upregulated exhaustion-related TF gene examined in PRDM1 KO CAR T-cells after multiple episodes of antigen stimulation (FIG. 5B) and NR4A3 is significantly elevated in hypofunctional NR/PR CLL patient CD19 CAR T-cells (FIG. 12B), both NR4A3 and PRDM1 were knocked out (FIG. 12C) and the gene-edited CAR T-cells were functionally characterized. While NR4A3 single KO CAR T-cells exhibited a similar expansion level as control CAR T-cells, PRDM1/NR4A3 dual KO CAR T-cells exhibited the highest level of antigen-induced proliferative capacity (FIG. 5C). The frequency of CAR T-cells expressing cytotoxic perforin and granzyme molecules was also measured. NR4A3 PRDM1 double KO partially restored cytotoxic function in the setting of perform and granzyme expression in CD8⁺CAR⁺ T-cells (FIG. 12D). PRDM1/NR4A3 KO CAR T-cells also exhibited increased frequencies of CCR7, CD62L, and TCF1 (encoded by TCF7) expressing cells compared to PRDM1 single KO CAR T-cells (FIGS. 12E, 12F).

Because CD4⁺ CAR T-cells are important to antitumor efficacy, the impact of the various KOs on CD4⁺ CAR T-cells was also determined with respect to differentiation phenotypes, as well as cytotoxic molecule expression during the aforementioned in vitro stress tests. Similar to CD8⁺ CAR T-cells, PRDM1 KO increased frequencies of CD4⁺ CAR T-cells expressing early memory markers such as CCR7 and TCF7 (FIG. 12G). However, combined KO of PRDM1 and NR4A3 had a stronger effect on rescuing expression of cytotoxic perform and granzyme molecules in CD4⁺ CAR T-cells compared to CD8⁺ T-cells (FIG. 12H).

Next, effector functions were assessed in the context of PRDM1/NR4A3 KO and it was discovered that PRDM1/NR4A3 KO CAR T-cells maintained elevated levels of effector cytokine production after multiple tumor challenges, whereas PRDM1 and NR4A3 single KO CAR T-cells showed similar IL-2 and TNFα secretion levels as control CAR T-cells (FIGS. 5D, 5E). This potency enhancement conferred by PRDM1 and NR4A3 dual KO was consistent in the context of both high and low PSMA expression (FIGS. 121, 12J). Furthermore, unlike PRDM1 single KO CAR T-cells which lost cytotoxic function following serial stimulation, PRDM1/NR4A3 KO CAR T-cells displayed sustained killing activity over time (FIGS. 5F, 5G).

To determine whether PRDM1/NR4A3 KO CAR T-cells exhibit aberrant growth patterns potentially indicative of transformation, it was tested whether CAR T-cell proliferation is antigen-dependent using irradiated PC3 cells with and without PSMA expression as stimuli. Notably, PRDM1/NR4A3 KO CAR T-cells failed to expand, accompanied by a reduction in viability when co-cultured with PSMA-negative PC3 cells, suggesting that cell expansion and survival is antigen-dependent (FIG. 5H).

### Example 7: Upregulation of Exhaustion-Related Transcription Factors in PRDM1 KO CAR T-Cells is Attributed to Increased Chromatin Accessibility and Calcineurin-NFAT Signaling

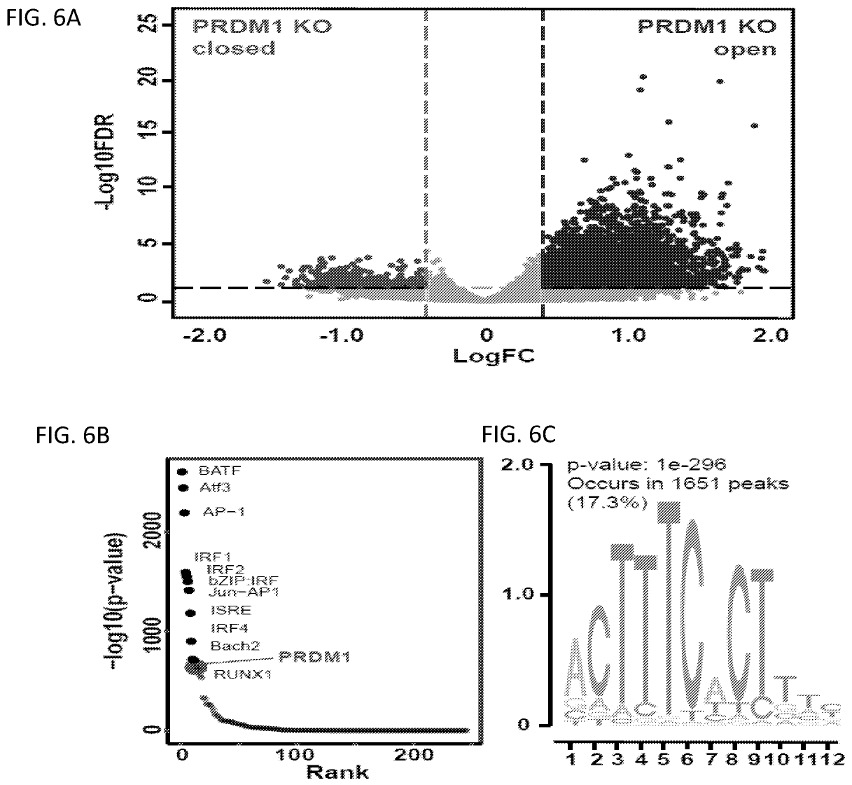

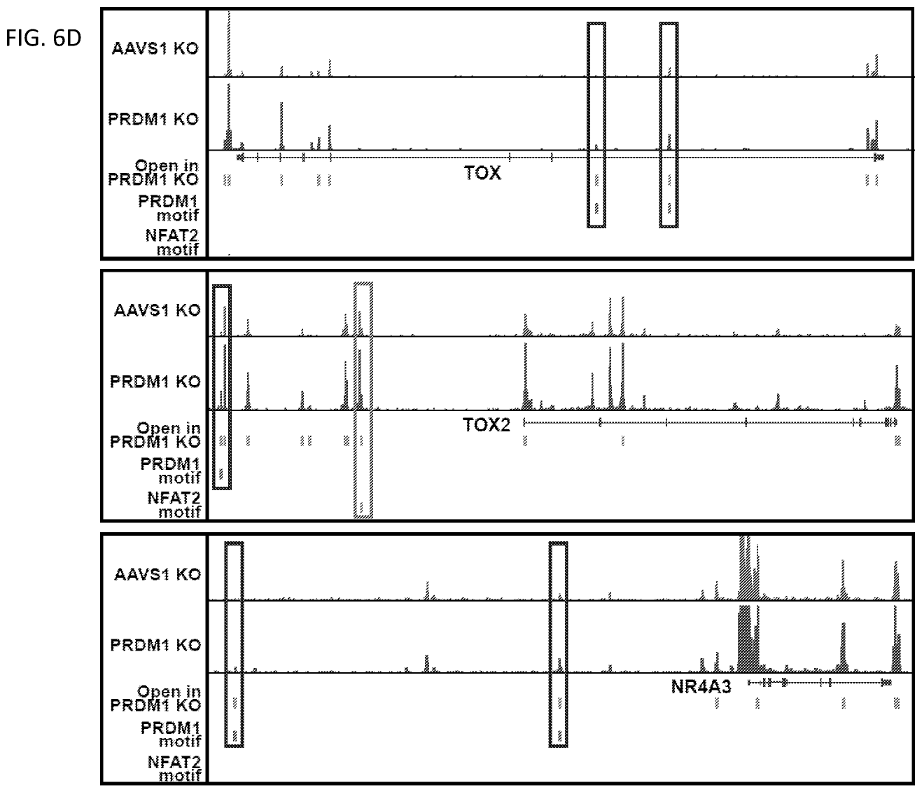

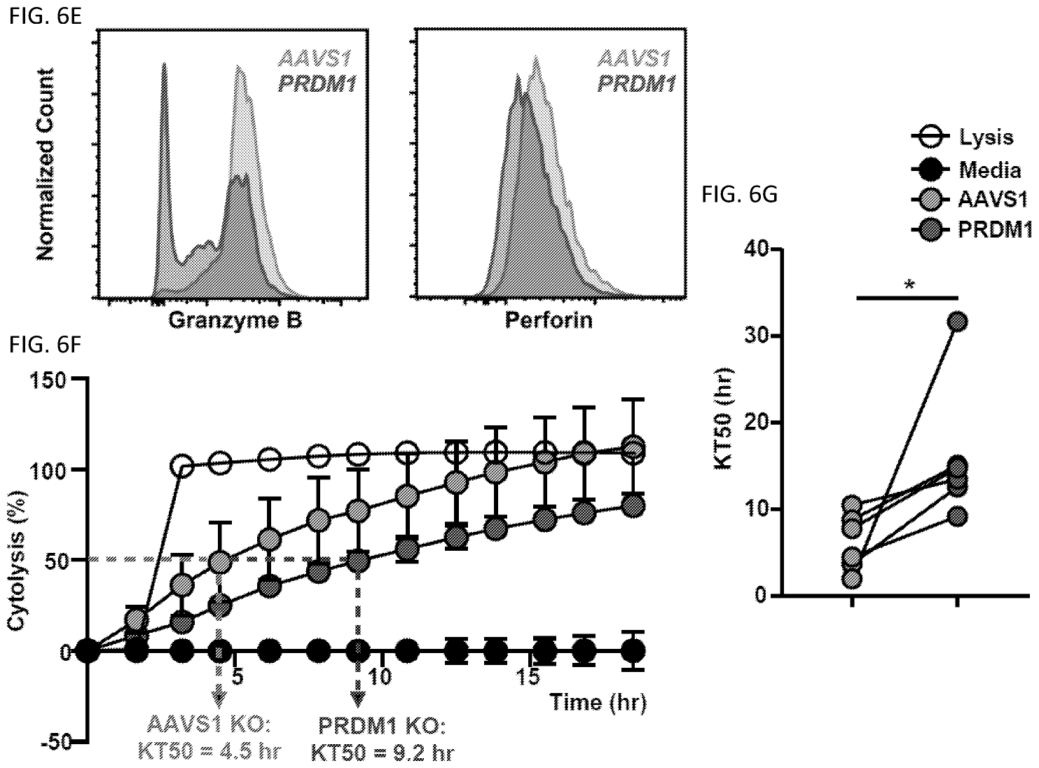

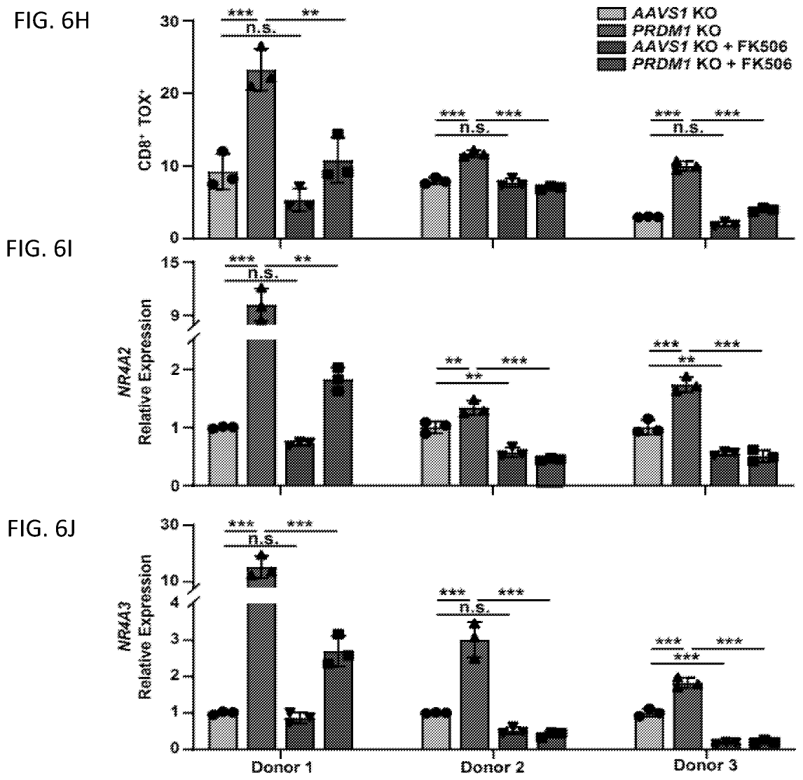

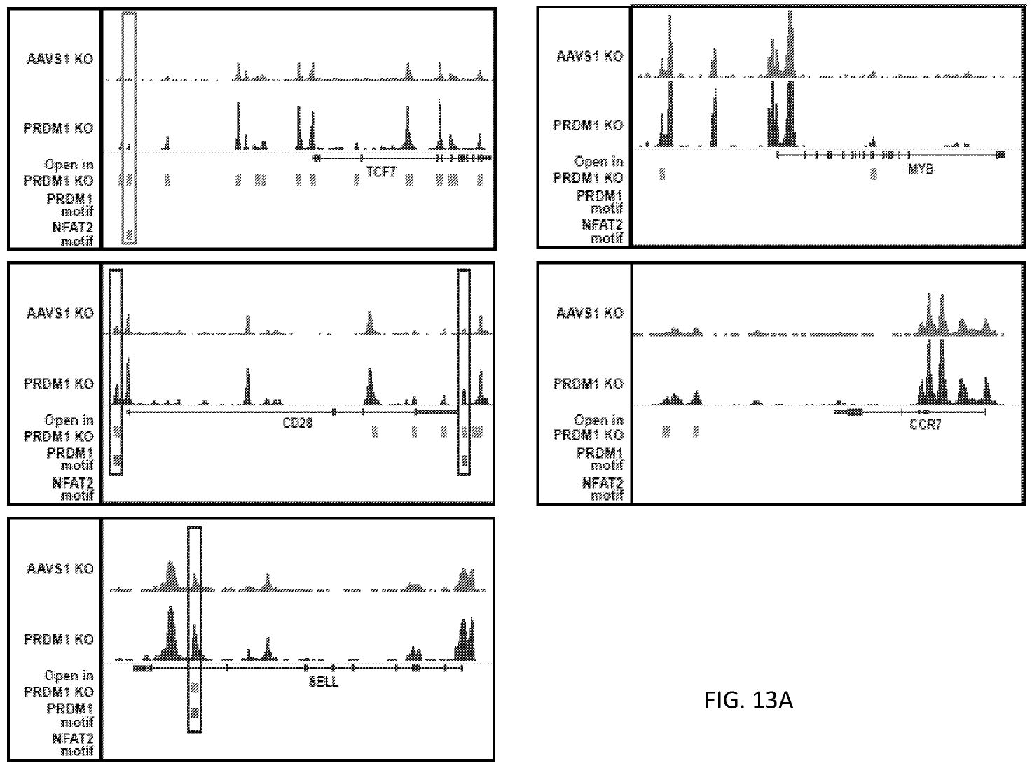

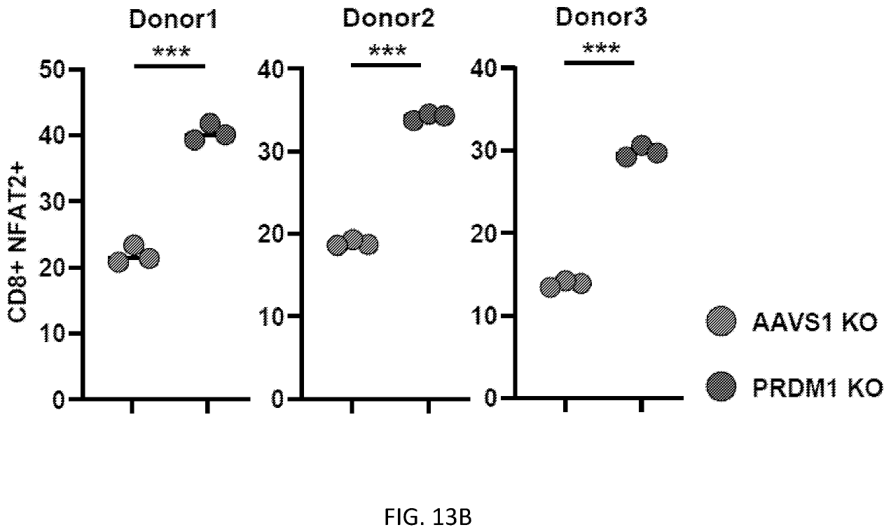

Next, the mechanism of exhaustion in TFs upregulation in PRDM1 KO CAR T-cells was investigated. Emerging evidence shows that PRDM1 epigenetically regulates transcription of memory-related genes by directly binding to the promoter regions and recruiting histone modifiers. It was investigated whether PRDM1 KO affects chromatin accessibility of exhaustion TF genes. ATAC-seq was performed on control and PRDM1 KO CAR T-cells harvested at resting state. PRDM1 knockout significantly increased global chromatin accessibility (FIG. 6A). Transcription motif enrichment analysis revealed that a PRDM1 motif was one of the most significantly enriched TF binding sites in PRDM1 KO CAR T-cells (FIGS. 6B-6C), suggesting that PRDM1 can act as an epigenetic repressor. Consistent with previous chromatin immunoprecipitation sequencing (ChIP-seq) results PRDM1 KO increased chromatin accessibility at loci corresponding to early memory and sternness genes such as TCF7, CD28, CCR7, SELL, and MYB (FIG. 13A). Notably, even before tumor challenge, PRDM1 KO significantly increased chromatin accessibility of exhaustion TFs, including TOX, TOX2, and NR4A3, and a subset of these open regions colocalized with the PRDM1 motif. These observations indicated that PRDM1 contributes to epigenetic repression of a battery of exhaustion-associated TFs, and PRDM1 KO CAR T-cells are predisposed to upregulate exhaustion TFs.

Given that CAR-T dysfunction can be induced by prolonged exposure to cancer cells due to low cytotoxic activity, the effects of decreased cytotoxicity of PRDM1 KO CAR T-cells on regulation of exhaustion TFs was investigated. PRDM1 KO CAR T-cells significantly downregulated Granzyme B and Perform (FIGS. 6E, 12D, 12H). This decrease in cytotoxic proteins led to delayed tumor clearance, which caused PRDM1 KO CAR T-cells to be exposed to target cancer cells twice as long as control T-cells (FIGS. 6F-6G). Due to prolonged exposure to cancer cells, PRDM1 KO increased expression of NFAT2 (FIG. 12B). As TOX and NR4A families are induced by calcineurin-NFAT signaling, FK506 calcineurin inhibitor was used to examine involvement of NFAT in exhaustion TFs upregulation in PRDM1 KO CAR T-cells. FK506 treatment either completely or partially counteracted PRDM1 KO-mediated upregulation of TOX, NR4A2, and NR4A3 during restimulation, implying that PRDM1 KO induces upregulation of exhaustion-associated TFs via increased NFAT signaling (FIGS. 6H-6J).

### Example 8: PRDM1/NR4A3 Dual KO Enhances In Vivo Antitumor Activity by Preserving TCF1⁺ CD8 T-Cells and Increasing Effector Function

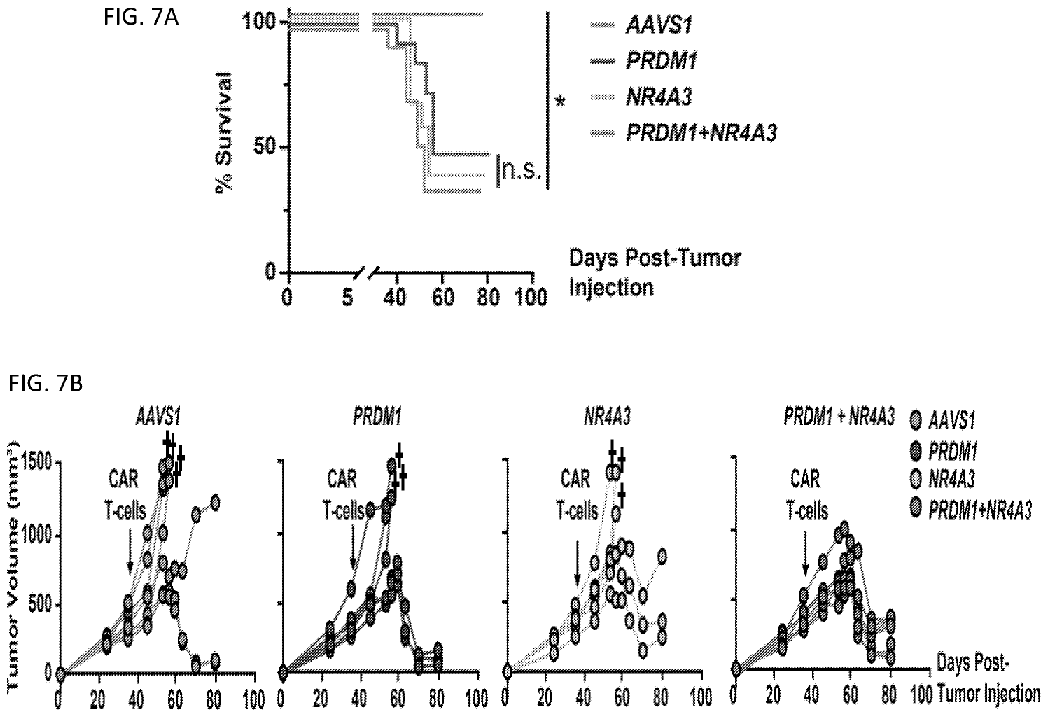

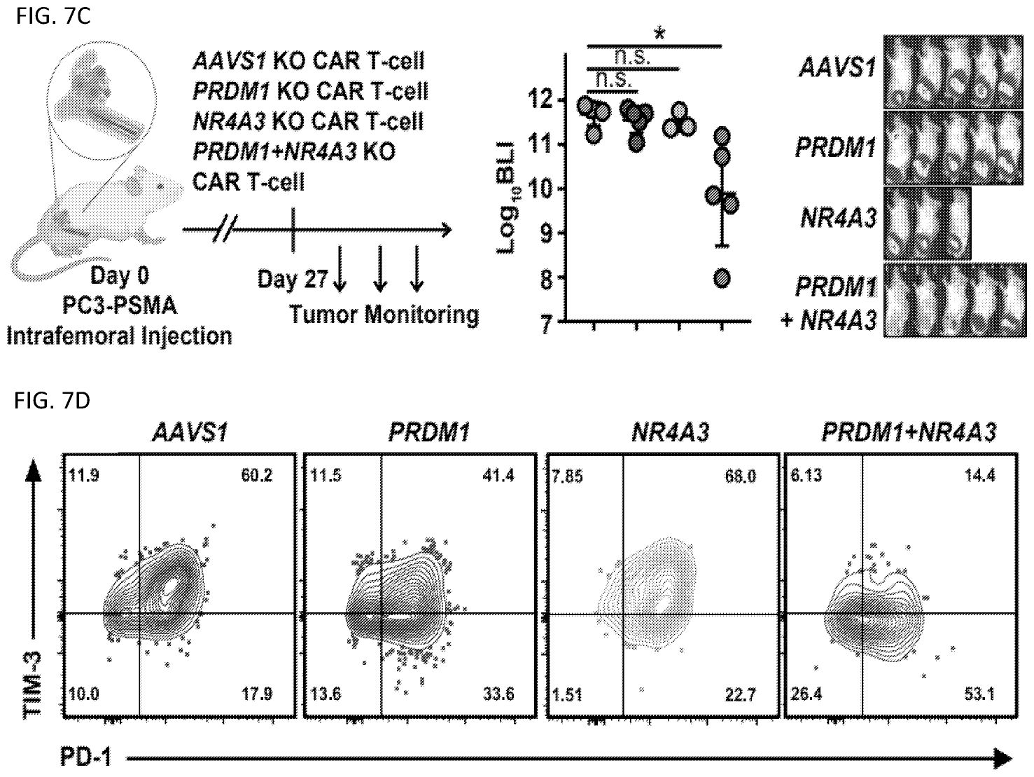

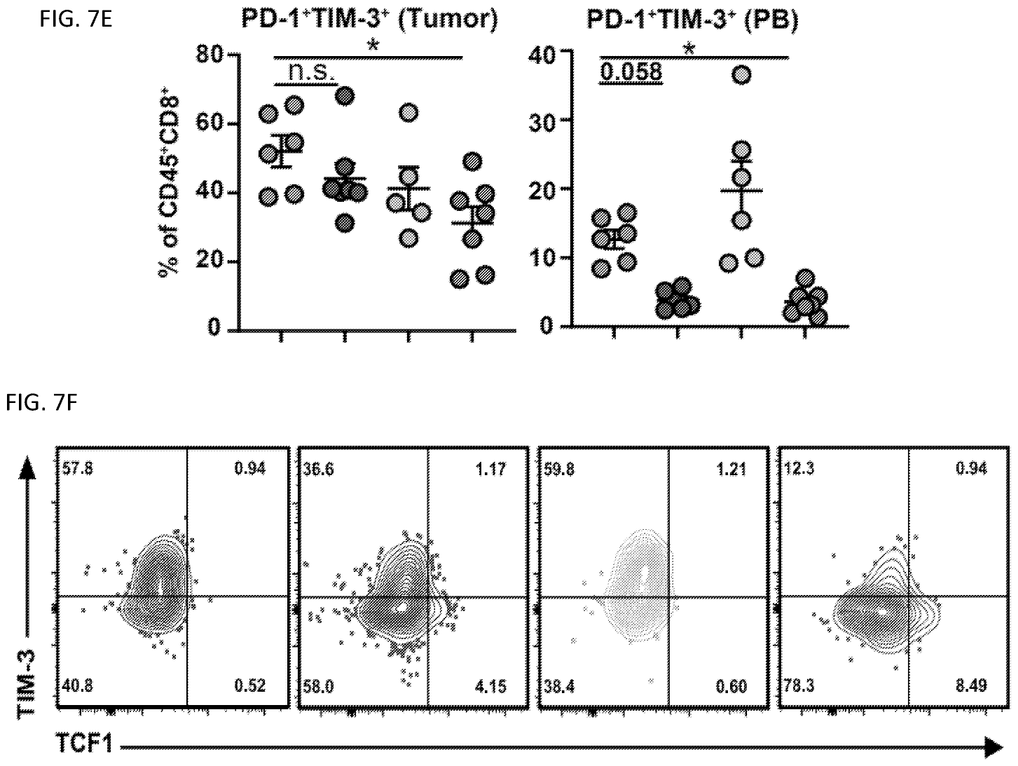

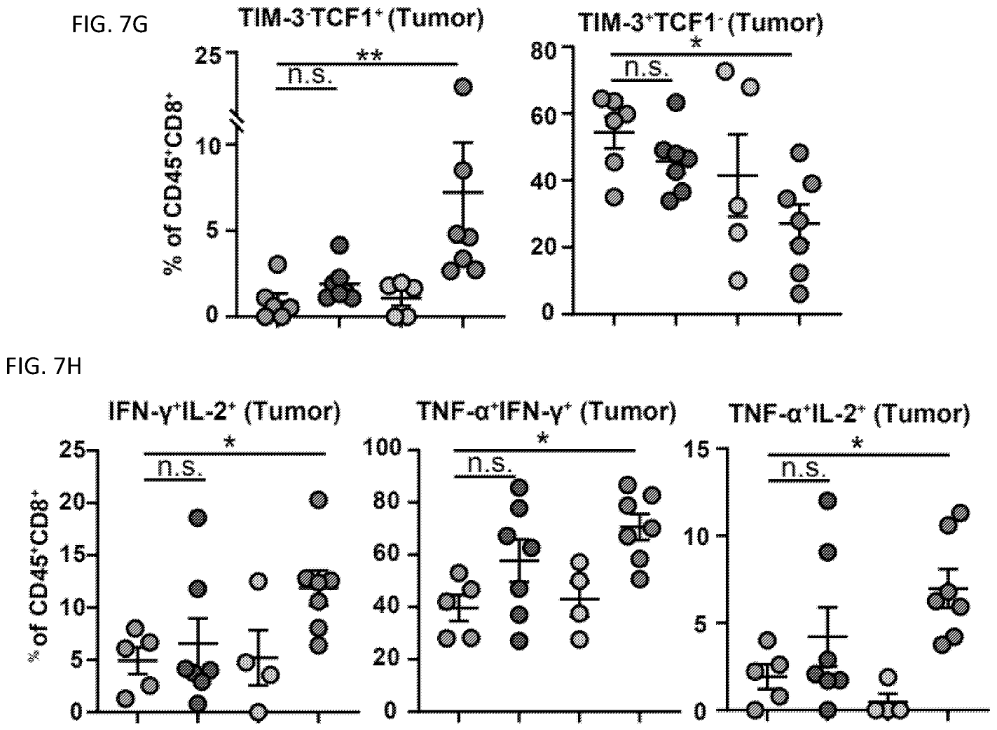

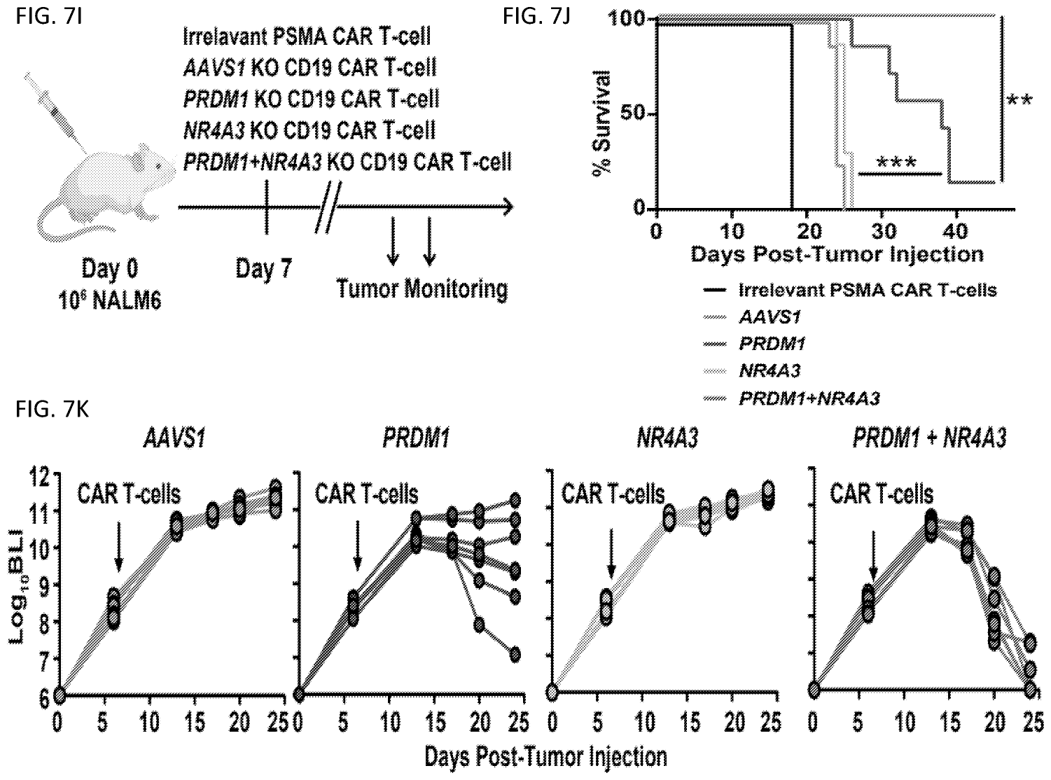

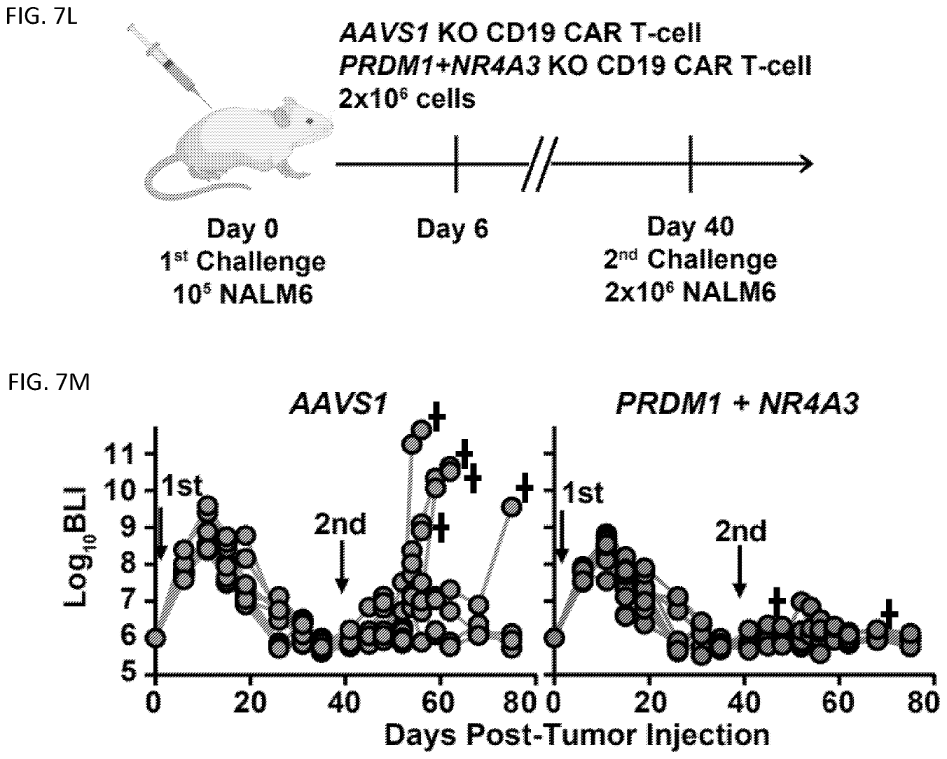

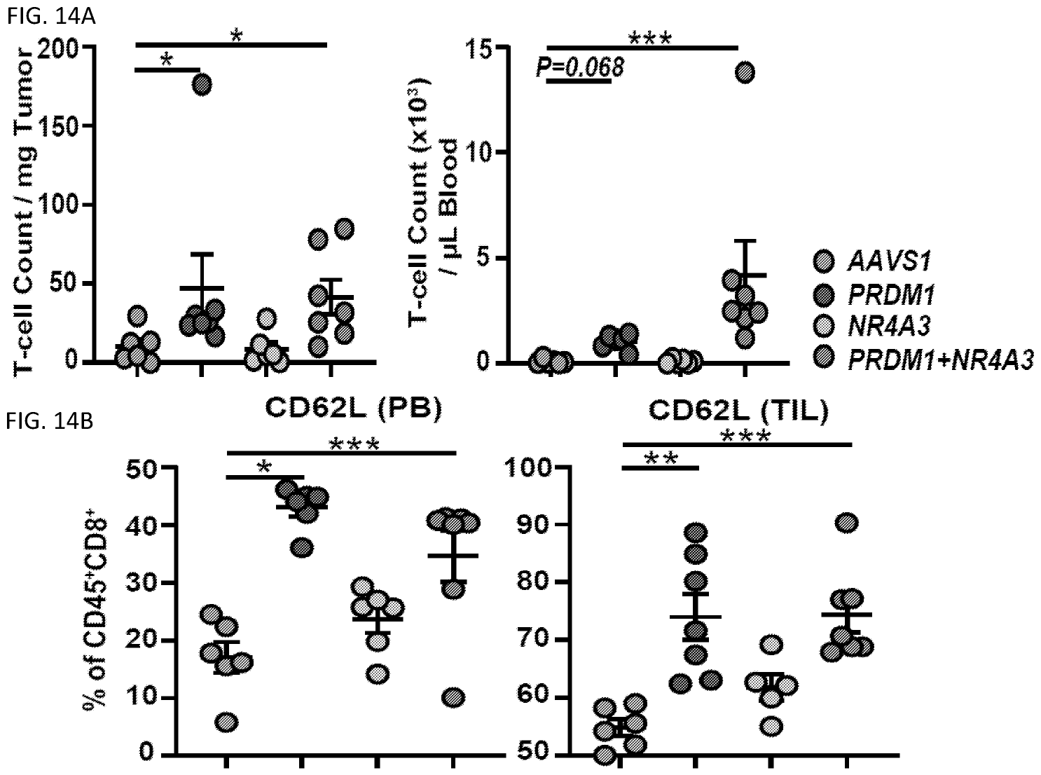

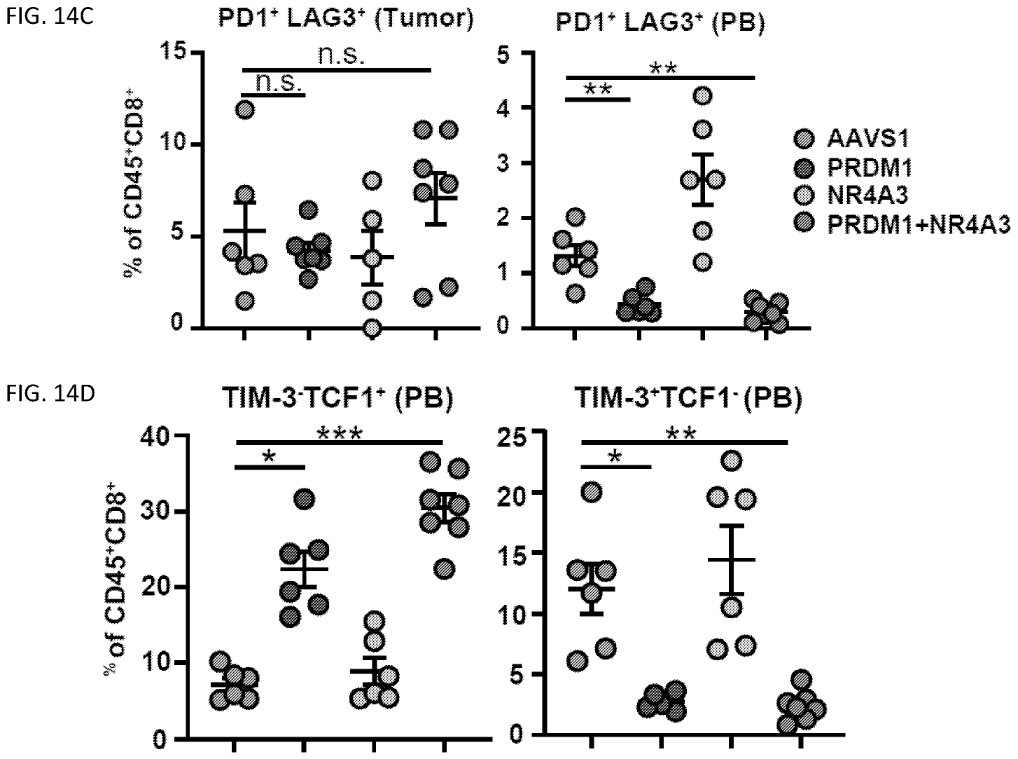

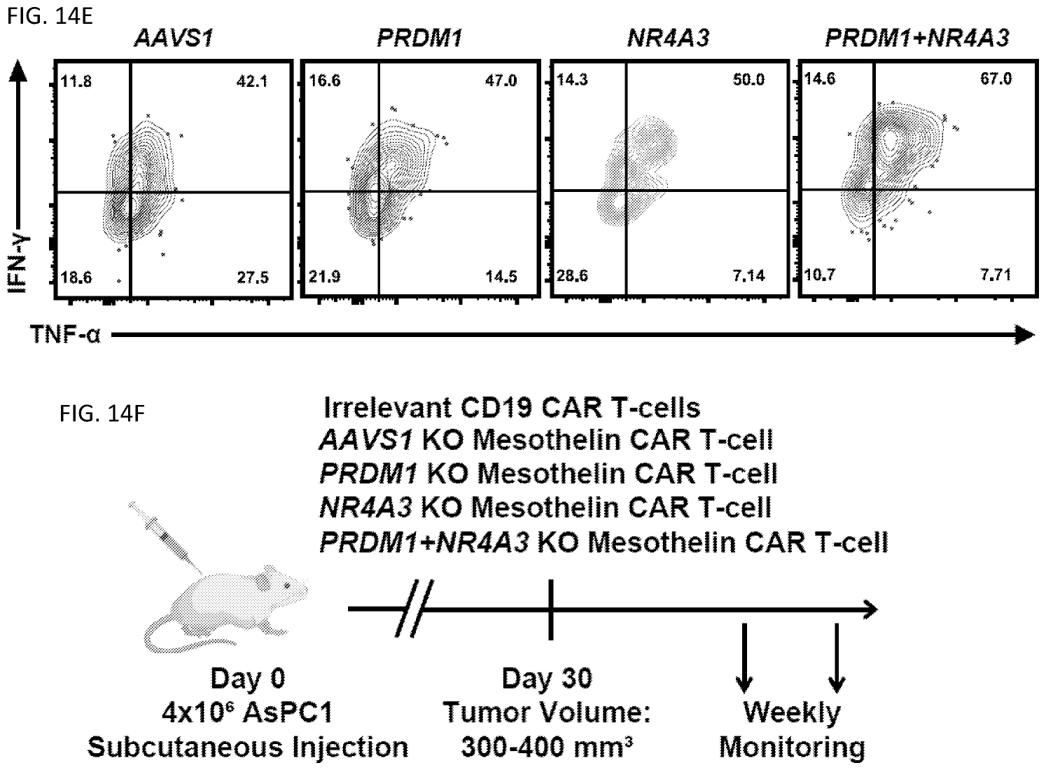

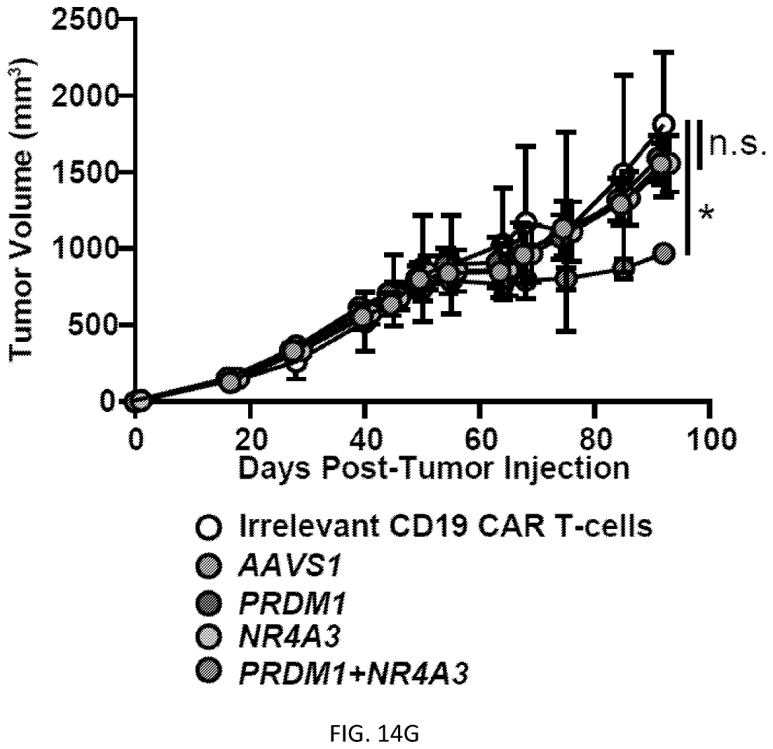

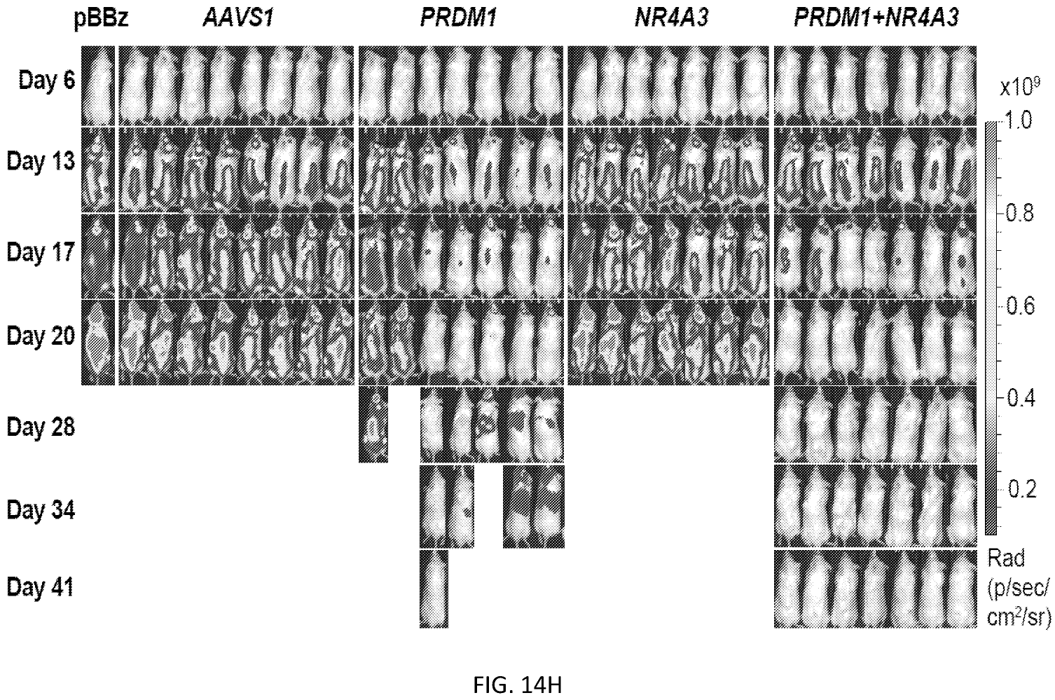

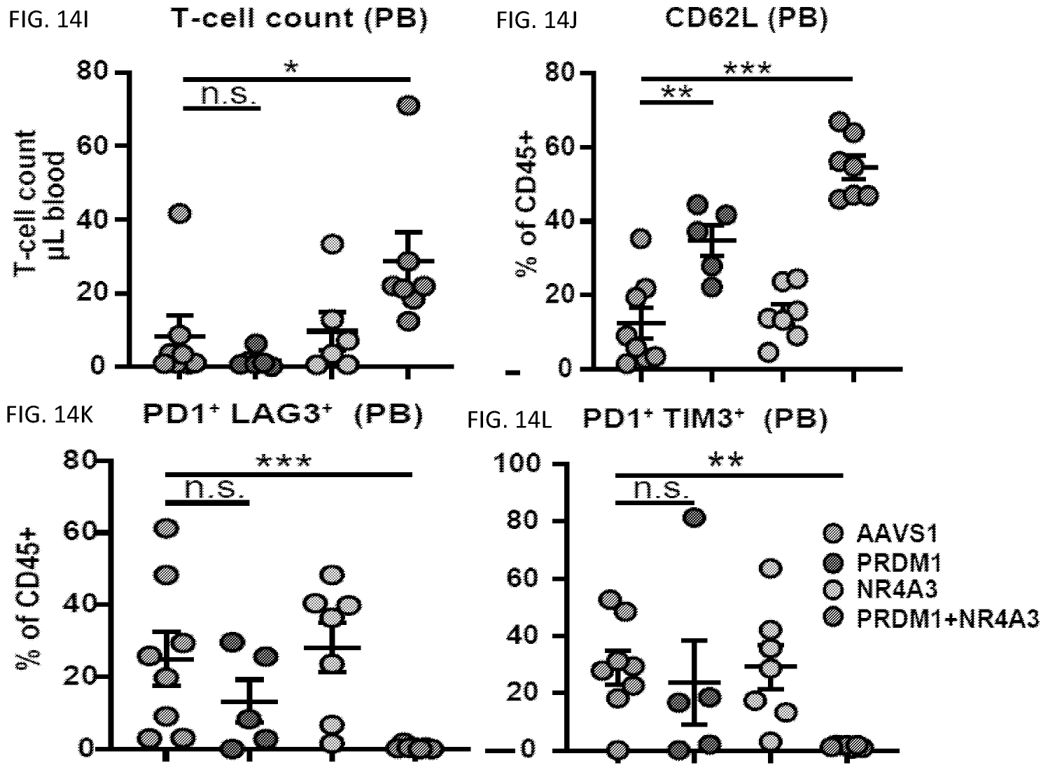

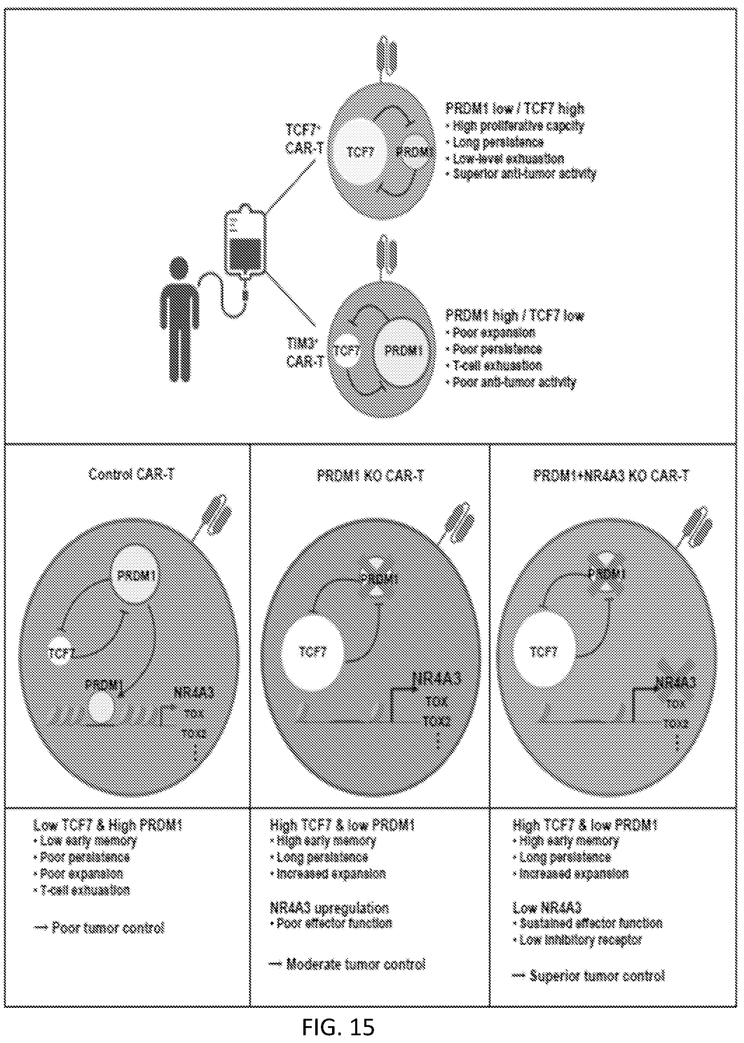

Based upon the evidence that PRDM1/NR4A3 dual KO sustains proliferative ability, effector functions, and early memory phenotype, it was asked whether PRDM1/NR4A3 KO CAR T-cells would exhibit enhanced antitumor activity in vivo. While AAVS1, PRDM1, and NR4A3 single knockout CAR T-cells controlled tumor growth in ˜50% of mice in the high tumor-burden PC3 xenograft model, PRDM1/NR4A3 double KO CAR T-cells successfully suppressed tumor growth in all mice in the group, in association with overall prolongation of survival (FIGS. 7A-7B). Because advanced prostate cancer commonly progresses within two years following the initiation of androgen-ablative therapy often with osseous in addition to visceral metastases, the approach was also tested in an intraosseous PC3-PSMA prostate tumor model. Bioluminescent tumor burden was also significantly decreased by a single infusion of PRDM1/NR4A3 KO PSMA CAR T-cells compared to PRDM1 and NR4A3 single KO as well as AAVS1 KO CAR T-cells in mice bearing intraosseous tumors generated by PC3-PSMA cell engraftment (FIG. 7C). Tumor and blood samples were subsequently collected to characterize the immunophenotype of CAR T-cells. Both PRDM1 single KO and PRDM1 NR4A3 double KO significantly increased the absolute numbers of CAR T-cells in the tumor and peripheral blood (FIG. 14A). This increased T-cell number may be due in part to the elevated frequencies of early memory T-cells with optimal proliferative potential observed with PRDM1 KO (FIG. 14B), which is consistent with previous results. To investigate how PRDM1 and NR4A3 depletion affect T-cell dysfunction, the frequencies of inhibitory receptor-expressing CAR T-cells were evaluated. While PRDM1 single KO CAR T-cells demonstrated reduced PD-1, TIM-3 and LAG-3 expression in the peripheral blood, their inhibitory receptor expression was comparable to AAVS1 KO CAR T-cells in the tumor where CAR T-cells receive persistent antigen stimulation (FIGS. 7D, 7E, 14C). In contrast, PRDM1/NR4A3 KO CAR T-cells demonstrated a significant reduction in the proportion of PD1⁺TIM3⁺ CD8 T-cells in both the tumor and peripheral blood (FIGS. 7D, 7E). Also, as observed in vitro, PRDM1 KO led to a substantial increase in TCF1 (encoded by TCF7) expression in CAR T-cells in vivo. Both PRDM1 single KO and PRDM1 NR4A3 double KO CAR T-cells exhibited increased stem cell-like TIM3-TCF1⁺ CD8 T-cells in the tumor and peripheral blood, although only PRDM1 NR4A3 KO CAR T-cells showed a significant decrease in the frequencies of TIM3⁺ TCF1⁻ exhausted T-cells in the tumor (FIGS. 7F, 7G, 14D).

To investigate whether PRDM1 NR4A3 dual KO enhances CAR TIL effector function, these TILs were reactivated ex vivo and intracellular cytokine production assessed. Consistent with in vitro results displayed in FIGS. 5D-5E, PRDM1 or NR4A3 single KO failed to improve effector cytokine production, whereas PRDM1 NR4A3 double KO CAR T-cells maintained higher polyfunctionality compared control CAR T-cells (FIGS. 7H, 14E). Antitumor activity of PRDM1 NR4A3 KO CAR T-cells was also evaluated in the NALM6 ALL model. Similar to the result from PC3-PSMA model, NR4A3 single KO CD19 CAR T-cells failed to suppress tumor growth (FIGS. 7I-7K). PRDM1 single KO moderately enhanced tumor control and survival, however, it was only when PRDM1 KO was combined with NR4A3 KO that CAR T-cells induced rapid tumor clearance and durable anti-tumor activity (FIGS. 7I-7K). Further, the efficacy of PRDM1 and NR4A3 single KO or PRDM1 NR4A3 double KO mesothelin-directed CAR T-cells was assessed in an in vivo model of highly resistant pancreatic adenocarcinoma incorporating AsPC1 tumor cells expressing endogenous levels of mesothelin (FIG. 14F). In this study, only PRDM1 NR4A3 double knockout anti-mesothelin CAR T-cells mediated a significant reduction in tumor burden over time (FIG. 14G).

Additionally, in association with robust antitumor activity (FIG. 14H), PRDM1 NR4A3 KO enhanced CD19 CAR T-cell expansion as well as central memory T-cell differentiation and reduced proportions of peripheral blood CAR T-cells co-expressing multiple inhibitory receptors (FIGS. 14I-14L). To evaluate the durability of PRDM1 NR4A3 double KO CAR T-cell therapeutic efficacy, a study was conducted in which CAR T-cell-treated NSG mice were rechallenged with NALM-6 cells 40 days after the initial leukemia cell transfer. Because of the aggressive nature of this model, a relatively high dose of CAR T-cells was administered across all groups during the primary challenge to ensure initial tumor clearance. As shown in FIGS. 7L-7M, PRDM1 NR4A3 dual KO CAR T-cells demonstrated better control of tumor growth than control CAR T-cells following rechallenge. Taken together, these findings suggest that PRDM1 depletion increases CAR-T expansion and mitigates dysfunction by increasing TCF7⁺ CD8 T-cells and decreasing TIM3⁺ CD8. Moreover, the results demonstrate that NR4A3 KO further improves in vivo anti-tumor activity by reducing exhaustion and inducing durable effector function.

### Example 9: Discussion

Full therapeutic potential of CAR-T therapies has been limited due to poor expansion, persistence, and lack of durable effector function, which are often the result of low early memory and high exhausted T-cell populations in infusion products. The present study offers a novel engineering strategy that enriches desirable, and depletes undesirable, CAR-T populations identified in the infusion products.

In the scRNA-seq analysis of PSMA CAR-T infusion products, stem cell-like TCF7⁺ CD8 and exhausted-like TIM3⁺ CD8 were identified. A series of works revealed that TCF7⁺ CD8 T-cells are a discrete CD8 population that expands and generates cytotoxic progenitors in response to checkpoint blockade therapies, which are critical for anti-tumor activity. In addition, in CAR T-cells, TCF7 regulon is associated with long-term persistence and durable response, which is in line with previous studies suggesting that TCF7 mediates persistence of CD8 effector T-cells and differentiation of central memory T-cells. In contrast, TCF7-TIM3⁺ marks exhausted T-cells and is associated with poor clinical response to checkpoint inhibitors. It was shown herein that the TCF7⁺ CD8 population in PSMA CAR-T infusion products enriched gene signatures that are associated with stem cell-like T-cells and durable response to CAR-T therapy. In sharp contrast, TIM3⁺ CD8 T-cells with low TCF7 expression showed high exhaustion, IFN response, poor persistence, and poor CAR-T response scores. In agreement with these observations, an infusion product with high TCF7 and low TIM3 score exhibited improved PSA response and expansion compared infusion products with low TCF7 and high TIM3 score, highlighting the relevance of this population in prostate cancer.

Unlike TCF7T CD8 cells, TIM3⁺ CD8 cells express a high level of PRDM1, which is known to negatively regulate TCF7 and mediate terminal differentiation and exhaustion. Herein, CRISPR/Cas9-mediated knockout of PRDM1 not only successfully depleted the TIM3⁺ CD8 signature and increased the TCF7⁺ CD8 population in infusion products, but also substantially increased TCF1⁺TIM3⁻ CD8 T-cells in the tumor and peripheral blood in a castration-resistant mouse model.

PRDM1 regulates effector function of CD8 T-cells and is required for GZMB expression. Previous mouse studies have shown that despite significant reduction in the cytotoxic molecule expression, the cytolytic activity of PRDM1 KO CD8 T-cells was marginally affected and both WT and PRDM1 KO CD8 T-cells successfully cleared the virus in an acute LCMV infection model. On the contrary, in chronic LCMV infection, cytotoxic activity of PRDM1 KO CD8 T-cells was significantly impaired, suggesting that loss of PRDM1 can profoundly compromise cytotoxicity during exhaustion in which cytotoxic activity of CD8 T-cells is relatively low. Consistent with these previous studies, cytotoxicity of PRDM1 KO CAR T-cells was substantially compromised after multiple rounds of restimulation, which limited anti-tumor activity of PRDM1 KO CAR T-cells. It was found herein that deletion of exhaustion transcription factor, NR4A3, in PRDM1 KO CAR T-cells can rescue this cytotoxic defect and potentiate anti-tumor activity in vivo.

Until now, PRDM1 was presumed to induce exhaustion modules. Unexpectedly, it was found herein that PRDM1 KO predisposes CAR T-cells to upregulation of exhaustion TFs by increasing chromatin accessibility. In addition to this epigenetic regulation, PRDM1 KO increased calcineurin/NFAT signaling, which is known to be necessary and sufficient to induce exhaustion TFs. This increased NFAT signaling may be attributed to prolonged exposure of PRDM1 KO CAR T-cells to target cancer cells due to the delayed killing kinetics. These results suggest that PRDM1 can suppress expression of exhaustion TFs by directly or indirectly regulating chromatin accessibility and killing activity of CD8 T-cells.

In summary, this study provides a framework for developing next-generation CAR T-cells by identifying cellular features that are associated with clinical response and engineering CAR T-cells to enrich desirable, and deplete undesirable, populations in the product. This study highlights the fundamental role of PRDM1 in regulation of human T-cell memory and provides a deeper understanding of how PRDM1 regulates T-cell exhaustion. It was demonstrated that CRISPR/Cas9-mediated PRDM1+NR4A3 KO profoundly improved tumor control not only by enhancing CAR-T persistence and expansion but also by maintaining durable effector function during chronic stimulation, thereby tackling the major challenges for developing effective cellular immunotherapies.

### Example 9: PRDM1 Knock-Out

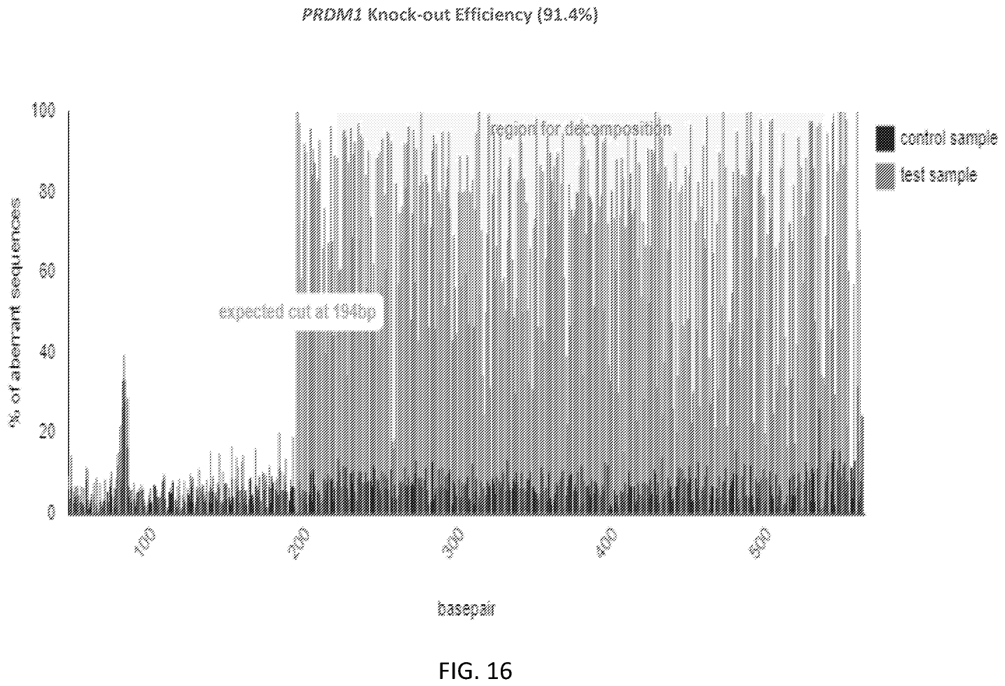

PRDM1 was knocked-out of primary human T cells using CRISPR/Cas9 (FIG. 16). TIDE analysis of Sanger sequencing data demonstrated a high degree of PRDM1 knock-out efficiency in bulk, primary human T cells using CRISPR/Cas9 technology (FIG. 16).

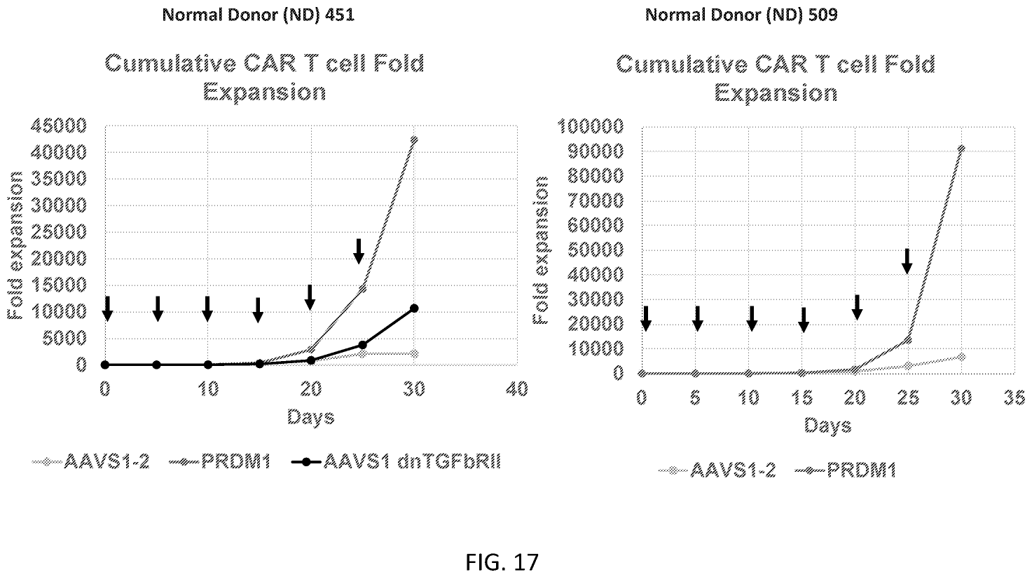

PRDM1 knock-out increased the proliferative capacity of CAR T cells (FIG. 17). An in vitro “stress test” was used to measure CAR T cell proliferative capacity. Anti-PSMA CAR T cells (used for proof-of-concept) were serially re-stimulated with PC3 (prostate) tumor cell targets (FIG. 17, indicated by black arrow) expressing PSMA to drive antigen-specific CAR T cell expansion. AAVS1-2 knock-out CAR T cells served as a negative control, while PSMA CAR T cells expressing a dominant-negative TGFβRII (known to enhance CAR T cell proliferation) served as a positive control (FIG. 17). Comprehensive flow cytometric immunophenotyping was done during the serial CAR T cell re-stimulation assay described in FIG. 17.

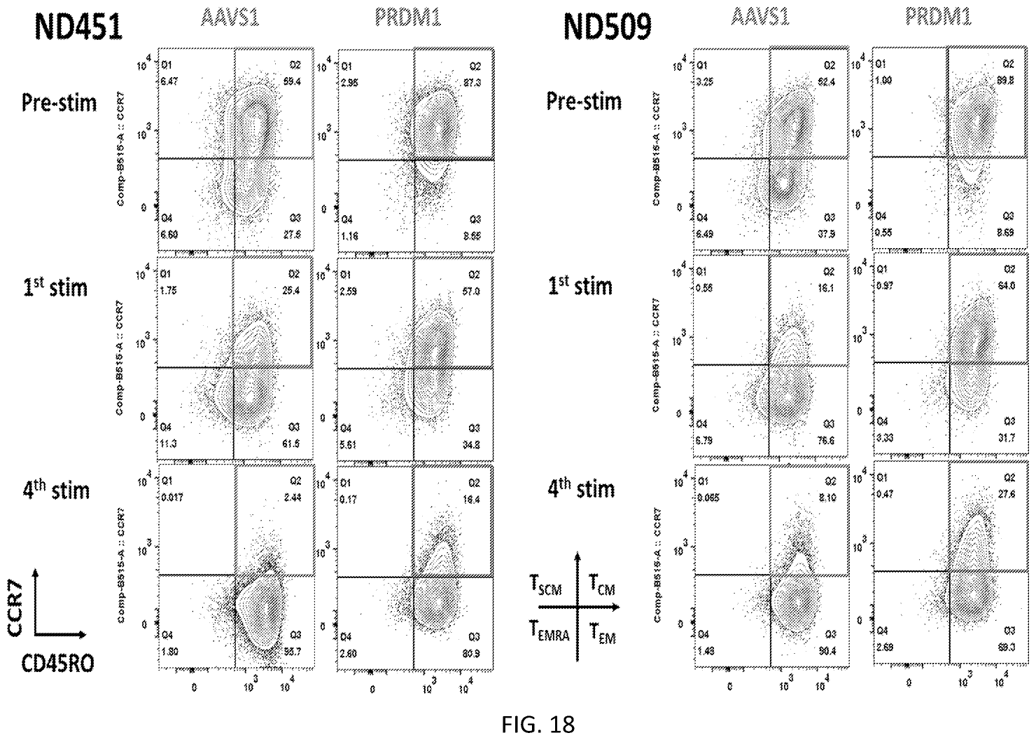

T cell differentiation was examined in the setting of PRDM1 knock-out using the markers, CCR7 and CD45RO. As shown in FIG. 18, PRDM1 knock-out maintains high frequencies of central memory (TCM; upper right quadrants) CAR T cells, which is the ideal differentiation state for adoptive cell therapy.

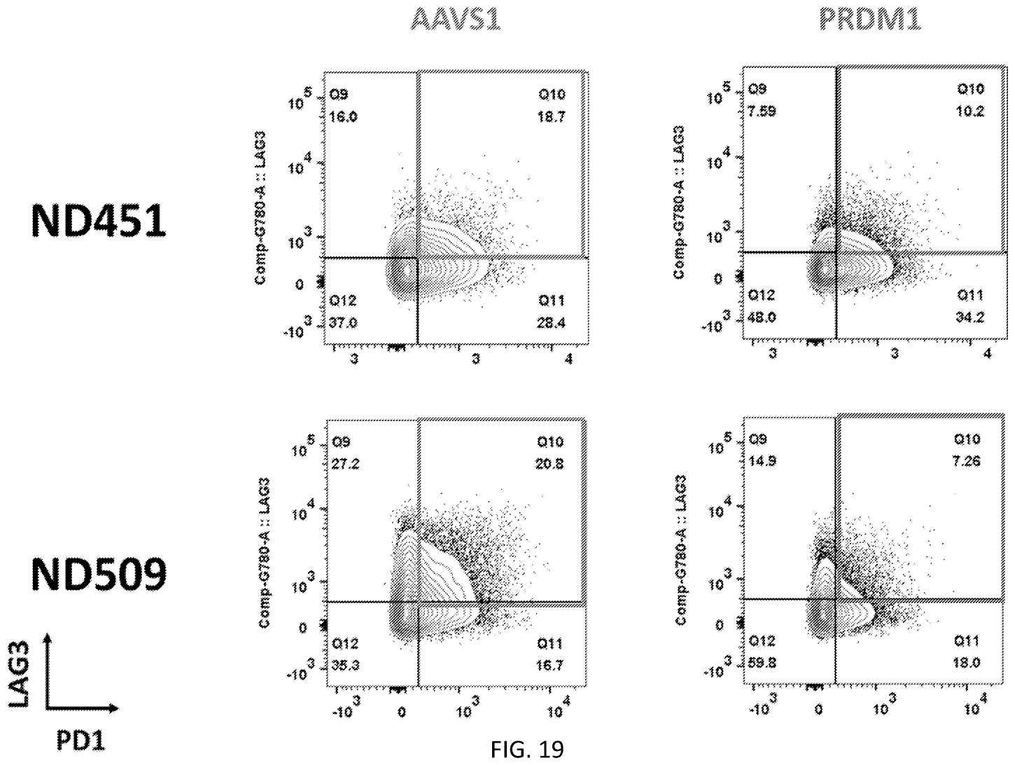

Comprehensive flow cytometric immunophenotyping was done during the serial CAR T cell re-stimulation assay described in FIG. 17. T cell inhibitory receptors/exhaustion markers were examined in the context of PRDM1 knock-out using phenotypes defined by PD-1 and LAG-3 expression. As shown in FIG. 19, PRDM1 knock-out decreases frequencies of CAR T cells expressing and co-expressing (FIG. 19, upper right quadrants) PD-1 and LAG-3. This exhaustion phenotype is shown following 4 rounds of PSMA CAR T cell stimulation with PC3 tumor targets.

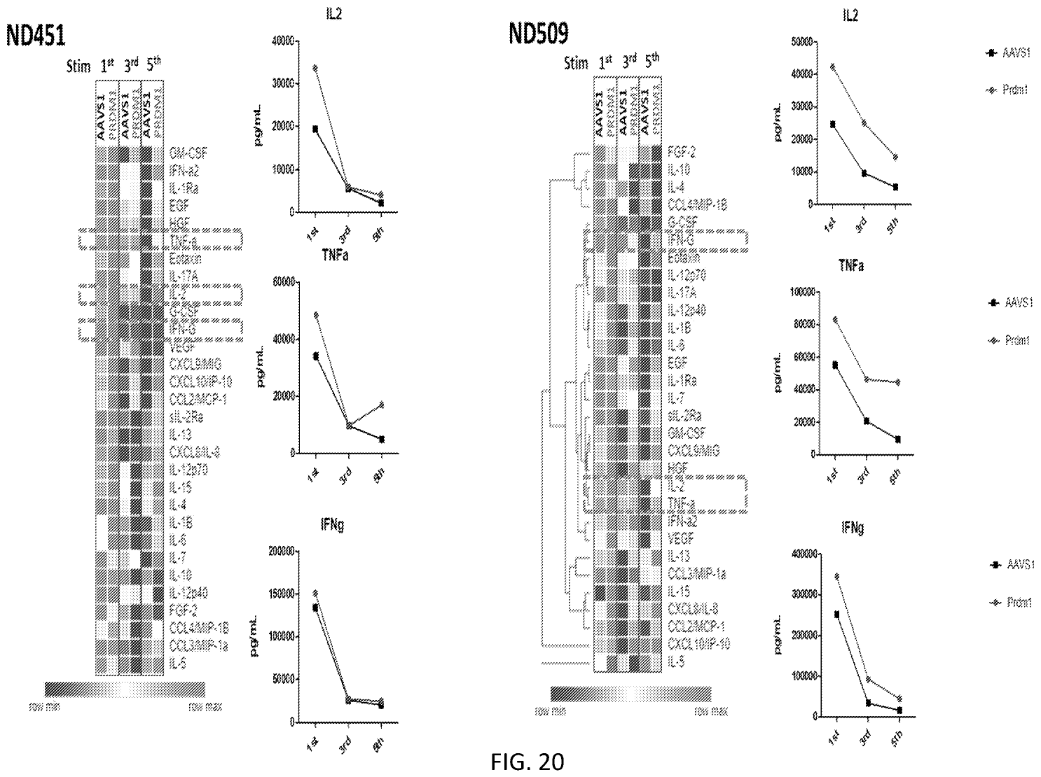

Multiplex cytokine analysis was conducted on supernatants from PRDM1 or AAVS (control) knock-out anti-PSMA CAR T cells serially restimulated with PC3 (prostate) tumor targets. As shown in FIG. 20, PRDM1 knock-out results in the enhancement/maintenance of CAR T cells expressing cytokines critical to anti-tumor responses, including tumor necrosis factor alpha (TNF-α), Interleukin-2 (IL-2) and interferon gamma (IFN-G).

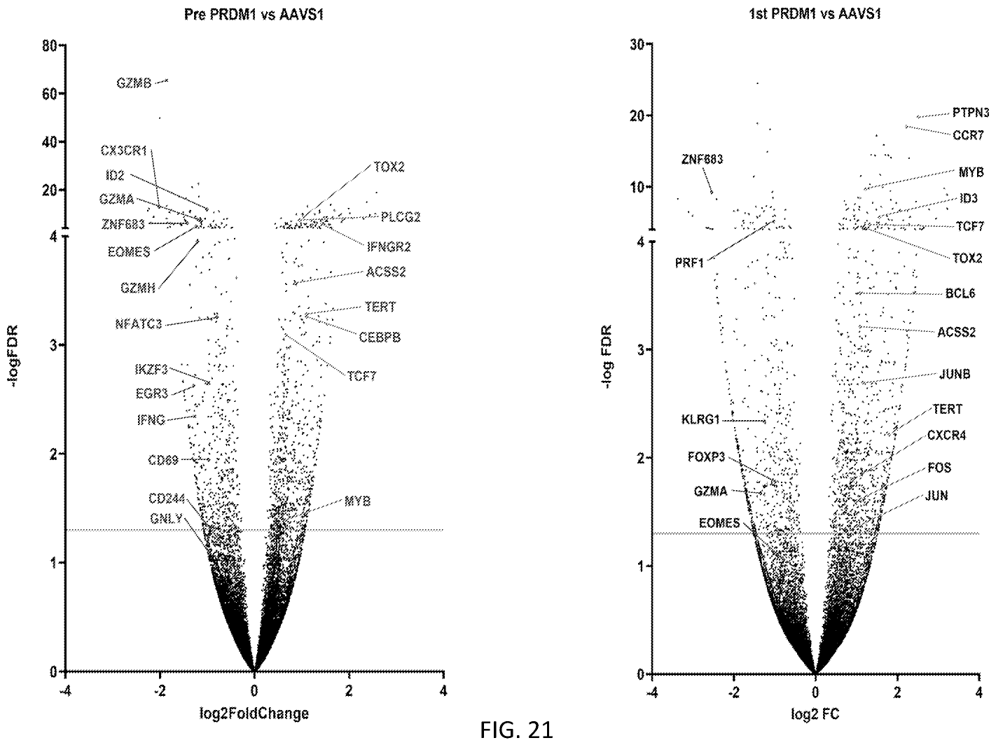

RNA-seq analysis was done on PRDM1 or AAVS1 (control) knock-out anti-PSMA CAR T cells prior to (Pre) and following the first stimulation (1st) with PC3 (prostate) tumor cells to determine the transcriptomic program conferred by Blimp-1 deficiency that increases CAR T cell anti-tumor potency. The volcano plots in FIG. 21 show downregulation of genes associated with terminal differentiation, exhaustion and senescence (left side of plots) and upregulation of genes (right side of plots) associated with early memory T cell formation/maintenance as well as stemness/self-renewal capacity.

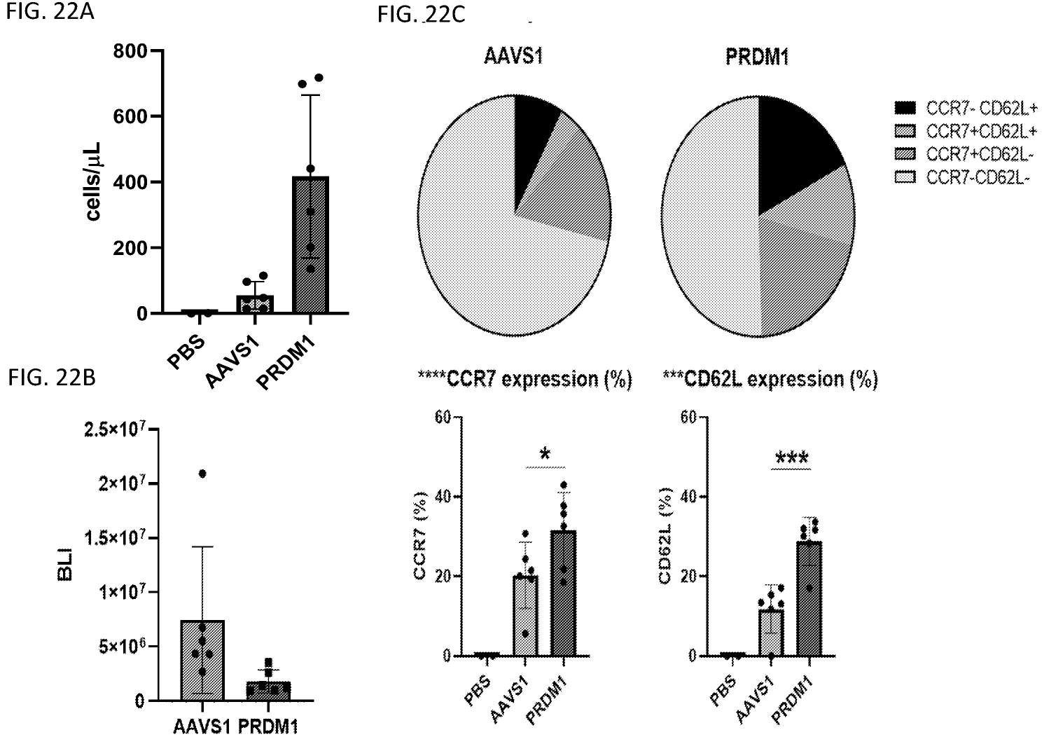

The in vivo anti-tumor efficacy of PRDM1 or AAVS (control) knock-out anti-PSMA CAR T cells is shown in FIGS. 22A-22C. NSG mice were injected subcutaneously with 1×10⁶ PC3-PSMA (prostate) tumor cells engineered to express luciferase. On day 27, when average tumor size reached ˜200 mm3, PSMA CAR T cells were injected intravenously. Results showed PRDM1 knock-out enhances the in vivo anti-tumor activity of CAR T cells in association with increased proliferative capacity and early memory differentiation.

### Example 10: Double Knock-Out of PRDM1 and TGFβRII

TGFβ signaling affects Blimp1 expression. TGFβ represses PRDM1 expression in human pan-T cells (Amina Dahmani et al., Cancer Immunol. Res., 2019) and in Murine Th1 cells (Christian Neumann et al., JEM, 2014). Once bioavailable TGFβ reaches the surface of the target cell, it binds a homodimer of TGFβ type II receptors (TGFβRII). Within the active receptor complex, the TGFβRII, which is a constitutively active kinase, undergoes autophosphorylation, as well as catalyzes transphosphorylation of the TGFβRI; transphosphorylation of the TβRI activates kinase activity. In the TGFβ pathway, Smad2 and Smad3 are receptor-regulated effector proteins (R-Smads), which are phosphorylated by the activated TGFβRI, resulting in R-Smad nuclear accumulation.

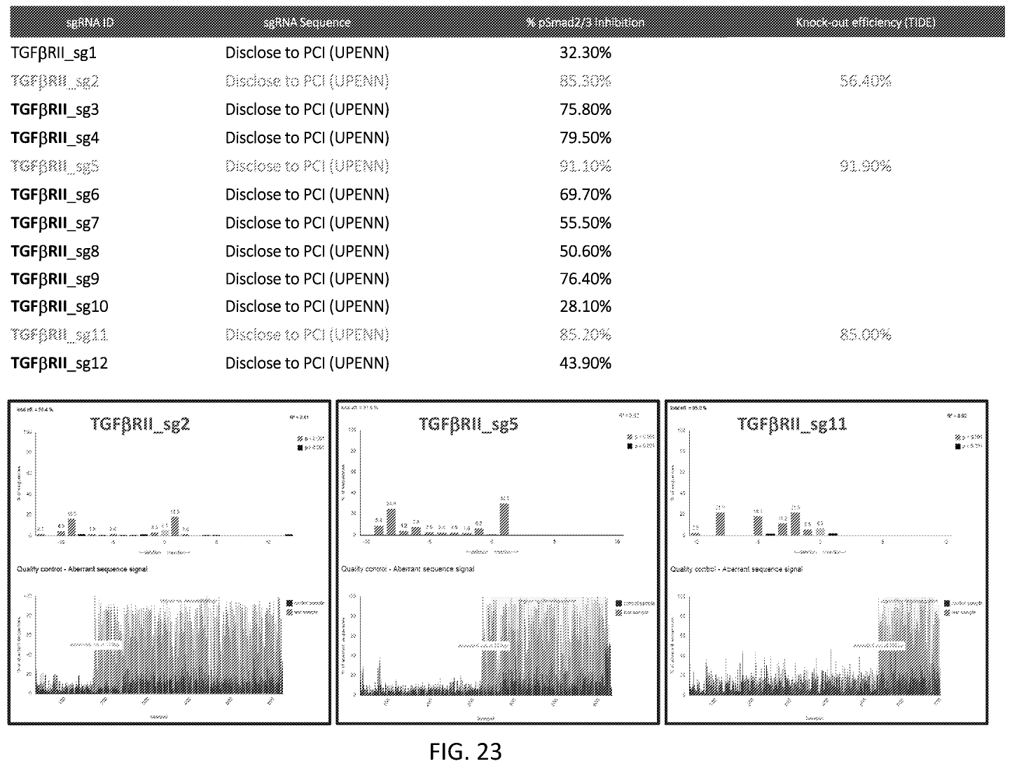

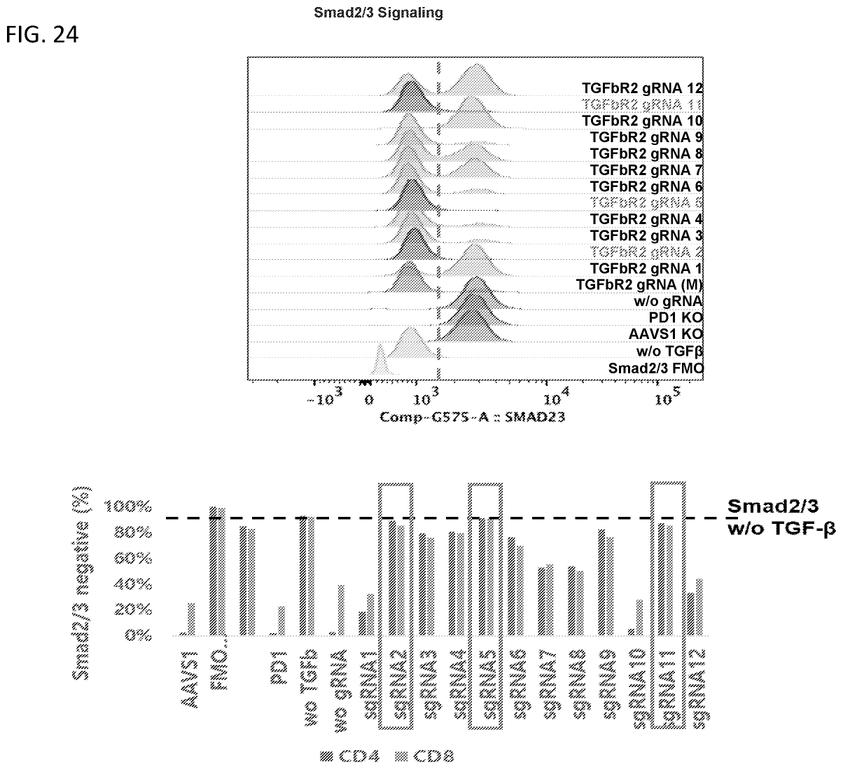

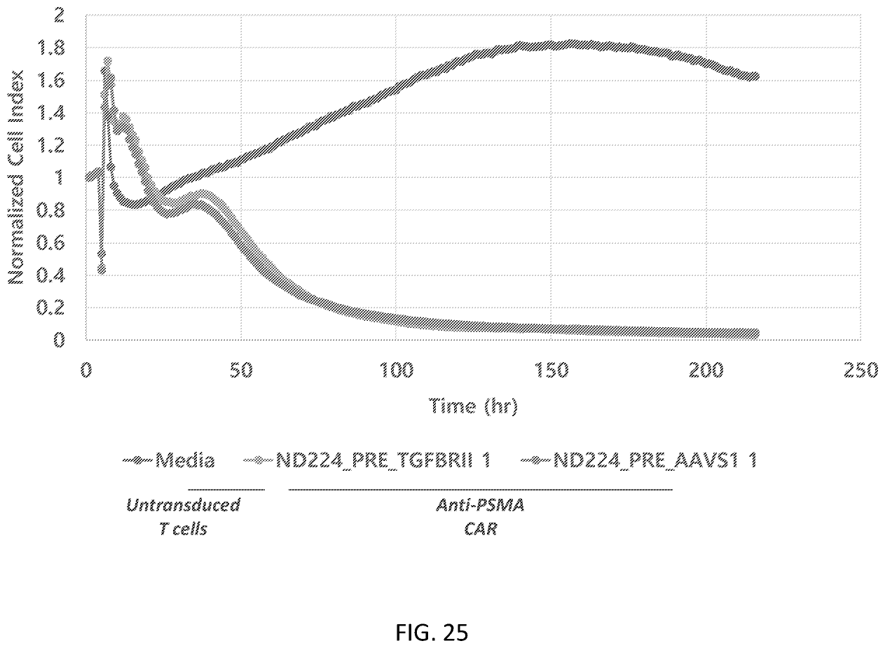

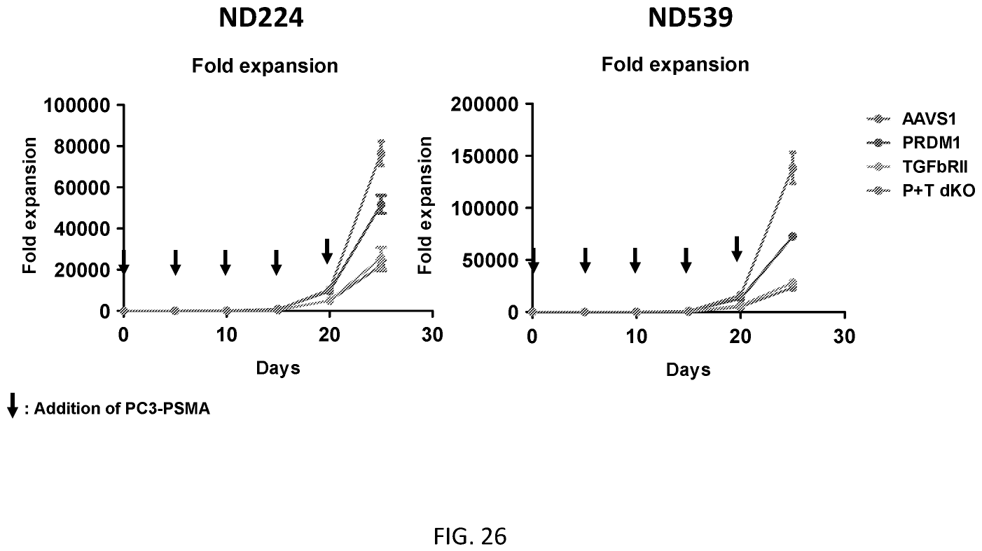

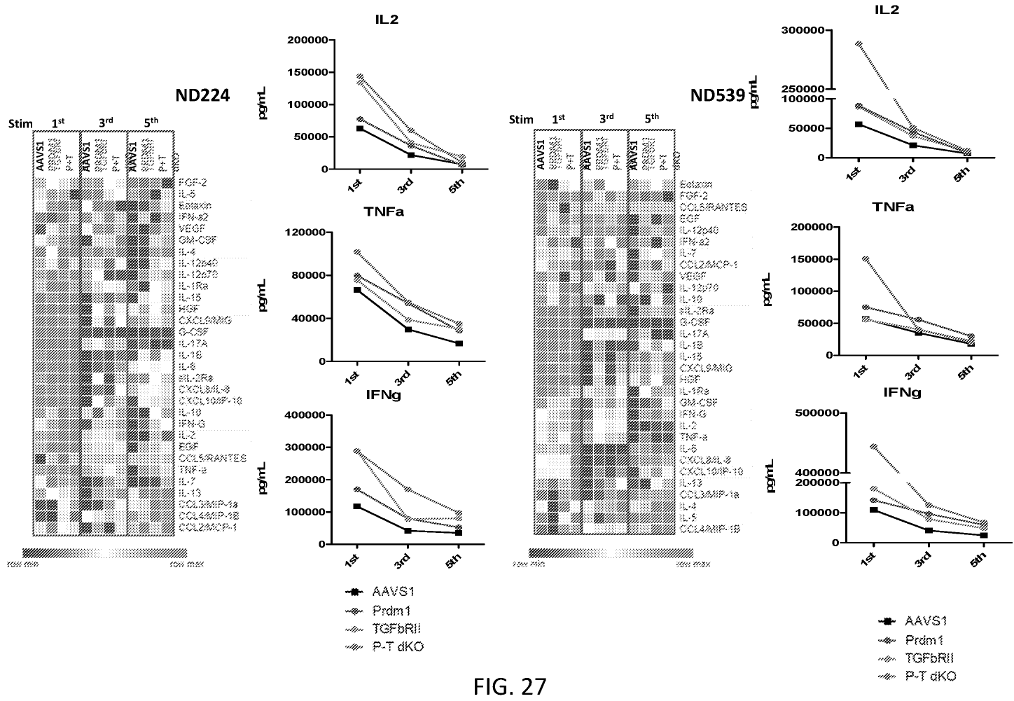

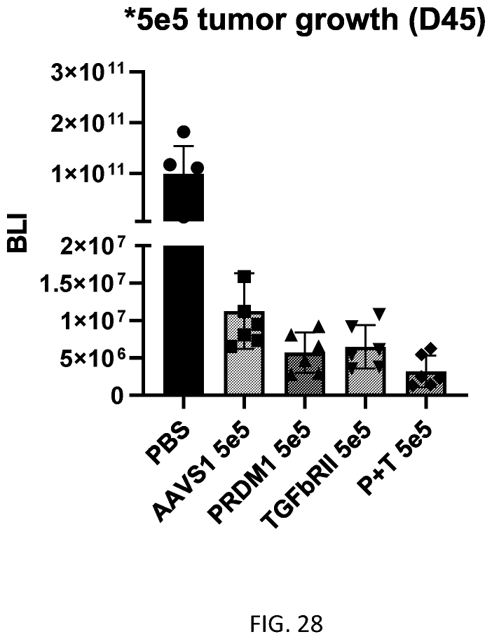

Endogenous TGFβRII was knocked-out (KO) of CAR T cells (FIG. 23). TGFβRII KO PSMA CAR T cells were cytotoxic (FIG. 25). However, double knock-out of PRDM1 and TGFβRII created a synergizing effect that enhanced the proliferative capacity of the CAR T cells (FIG. 26), enhanced cytokine production by the CAR T cells (FIG. 27), and enhanced in vivo CAR T cell anti-tumor efficacy (FIG. 28).

### ENUMERATED EMBODIMENTS

The following enumerated embodiments are provided, the numbering of which is not to be construed as designating levels of importance.

Embodiment 1 provides a modified immune cell or precursor cell thereof, comprising: a modification in an endogenous gene locus encoding PRDM1, wherein the modification is capable of downregulating gene expression of, or knocking out, endogenous PRDM1; a modification in an endogenous gene locus encoding NR4A3, wherein the modification is capable of downregulating gene expression of, or knocking out, endogenous NR4A3; and an exogenous T cell receptor (TCR) and/or chimeric antigen receptor (CAR) comprising affinity for an antigen on a target cell.

Embodiment 2 provides a modified immune cell or precursor cell thereof, comprising: a modification in an endogenous gene locus encoding PRDM1, wherein the modification is capable of downregulating gene expression of, or knocking out, endogenous PRDM1; and an exogenous T cell receptor (TCR) and/or chimeric antigen receptor (CAR) comprising affinity for an antigen on a target cell.

Embodiment 3 provides a modified immune cell or precursor cell thereof, comprising: a modification in an endogenous gene locus encoding PRDM1, wherein the modification is capable of downregulating gene expression of, or knocking out, endogenous PRDM1; a modification in an endogenous gene locus encoding TGFβRII, wherein the modification is capable of downregulating gene expression of, or knocking out, endogenous TGFβRII; and an exogenous T cell receptor (TCR) and/or chimeric antigen receptor (CAR) comprising affinity for an antigen on a target cell.

Embodiment 4 provides the modified immune cell or precursor cell of any preceding embodiment, wherein the modification comprises a CRISPR-mediated modification.

Embodiment 5 provides the modified immune cell or precursor cell of embodiment 4, wherein the CRISPR-mediated modification is introduced by a CRISPR system comprising a guide RNA that comprises a guide sequence that is sufficiently complementary with a target sequence in the endogenous gene locus encoding PRDM1, NR4A3 or TGFβRII.

Embodiment 6 provides the modified immune cell or precursor cell of claim 5, wherein the guide RNA comprises a nucleic acid sequence set forth in SEQ ID NO: 2 or SEQ ID NO: 4.

Embodiment 7 provides the modified immune cell or precursor cell of any preceding embodiment, wherein the modification is selected from the group consisting of a substitution, an insertion, a deletion, and an insertion/deletion.

Embodiment 8 provides the modified immune cell or precursor cell of any preceding embodiment, wherein the CAR comprises an antigen binding domain, a transmembrane domain, and an intracellular domain.

Embodiment 9 provides the modified immune cell or precursor cell of embodiment 8, wherein the antigen binding domain is capable of binding a tumor associated antigen (TAA).

Embodiment 10 provides the modified immune cell or precursor cell of claim 8, wherein the antigen binding domain is selected from the group consisting of an antibody, an scFv, and a Fab.

Embodiment 11 provides the modified immune cell or precursor cell of embodiment 8, wherein the transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

Embodiment 12 provides the modified immune cell or precursor cell of any preceding embodiment, wherein the CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

Embodiment 13 provides the modified immune cell or precursor cell of any preceding embodiment, wherein the CAR comprises an intracellular domain comprising an intracellular domain selected from the group consisting of cytoplasmic signaling domains of a human CD3 zeta chain, FcγRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

Embodiment 14 provides the modified immune cell or precursor cell of any preceding embodiment, wherein the antigen on a target cell is a tumor associated antigen (TAA).

Embodiment 15 provides the modified immune cell or precursor cell of any preceding embodiment, wherein the modified cell is resistant to cell exhaustion.

Embodiment 16 provides the modified immune cell or precursor cell of any preceding embodiment, wherein the modified cell is an autologous cell.

Embodiment 17 provides the modified immune cell or precursor cell of any preceding embodiment, wherein the modified cell is a cell isolated from a human subject.

Embodiment 18 provides the modified immune cell or precursor cell of any preceding embodiment, wherein the modified cell is a modified immune cell.

Embodiment 19 provides the modified immune cell or precursor cell of any preceding embodiment, wherein the modified cell is a modified T cell.

Embodiment 20 provides the modified immune cell or precursor cell of any preceding embodiment, wherein the modified cell is a gamma delta T cell.

Embodiment 21 provides the modified immune cell or precursor cell of any preceding embodiment, wherein the modified cell is a modified T cell resistant to T cell exhaustion.

Embodiment 22 provides a method of treating cancer in a subject in need thereof, the method comprising administering to the subject a composition comprising the modified immune cell or precursor cell thereof of any preceding embodiment.

Embodiment 23 provides a method for generating a modified immune cell or precursor cell thereof, comprising: introducing into an immune or precursor cell a CRISPR system comprising one or more polypeptides and/or nucleic acids capable of downregulating gene expression of endogenous PRDM1; and introducing into the immune or precursor cell a nucleic acid encoding an exogenous CAR and/or TCR, wherein the exogenous CAR and/or TCR comprises affinity for an antigen on a target cell.

Embodiment 24 provides a method for generating a modified immune cell or precursor cell thereof, comprising: introducing into an immune or precursor cell a CRISPR system comprising one or more polypeptides and/or nucleic acids capable of downregulating gene expression of endogenous PRDM1; introducing into an immune or precursor cell a CRISPR system comprising one or more polypeptides and/or nucleic acids capable of downregulating gene expression of endogenous NR4A3; and introducing into the immune or precursor cell a nucleic acid encoding an exogenous CAR and/or TCR, wherein the exogenous CAR and/or TCR comprises affinity for an antigen on a target cell.

Embodiment 25 provides a method for generating a modified immune cell or precursor cell thereof, comprising: introducing into an immune or precursor cell a CRISPR system comprising one or more polypeptides and/or nucleic acids capable of downregulating gene expression of endogenous PRDM1; introducing into an immune or precursor cell a CRISPR system comprising one or more polypeptides and/or nucleic acids capable of downregulating gene expression of endogenous TGFβRII; and introducing into the immune or precursor cell a nucleic acid encoding an exogenous CAR and/or TCR, wherein the exogenous CAR and/or TCR comprises affinity for an antigen on a target cell.

Embodiment 26 provides the method of any of embodiments 23-25, wherein the one or more polypeptides and/or nucleic acids capable of downregulating gene expression of endogenous PRDM1 introduces a CRISPR-mediated modification in an endogenous gene locus encoding PRDM1, and/or the one or more polypeptides and/or nucleic acids capable of downregulating gene expression of endogenous NR4A3 introduces a CRISPR-mediated modification in an endogenous gene locus encoding NR4A3, and/or the one or more polypeptides and/or nucleic acids capable of downregulating gene expression of endogenous TGFβRII introduces a CRISPR-mediated modification in an endogenous gene locus encoding TGFβRII.

Embodiment 27 provides the method of embodiment 25, wherein the modification is selected from the group consisting of a substitution, an insertion, a deletion, and an insertion/deletion.

Embodiment 28 provides the method of any of embodiments 23-27, wherein the CRISPR system comprises a CRISPR nuclease and a guide RNA.

Embodiment 29 provides the method of embodiment 28, wherein the CRISPR nuclease is Cas9.

Embodiment 30 provides the method of embodiment 28 or 29, wherein the CRISPR nuclease and the guide RNA comprise a ribonucleoprotein (RNP) complex.

Embodiment 31 provides the method of embodiment 30, wherein the RNP complex is introduced by electroporation.

Embodiment 32 provides the method of embodiment 28, wherein the guide RNA comprises a nucleic acid sequence set forth in SEQ ID NO: 2 or SEQ ID NO: 4.

Embodiment 33 provides the method of any of embodiment 23-30, wherein the nucleic acid encoding an exogenous CAR and/or TCR is introduced via viral transduction.

Embodiment 34 provides the method of embodiment 33 wherein the viral transduction comprises contacting the immune or precursor cell with a viral vector comprising the nucleic acid encoding an exogenous CAR and/or TCR.

Embodiment 35 provides the method of embodiment 34, wherein the viral vector is selected from the group consisting of a retroviral vector, a lentiviral vector, an adenoviral vector, and an adeno-associated viral vector.

Embodiment 36 provides the method of embodiment 35, wherein the viral vector is a lentiviral vector.

Embodiment 37 provides a method of treating cancer in a subject in need thereof, comprising administering to the subject modified immune or precursor cell generated by the method of claims 23-37.

Embodiment 38 provides a method of treating a disease or disorder in a subject in need thereof, comprising administering to the subject a modified T cell comprising: a modification in an endogenous gene locus encoding PRDM1, wherein the modification is capable of downregulating gene expression of, or knocking out, endogenous PRDM1; and an exogenous T cell receptor (TCR) and/or chimeric antigen receptor (CAR) comprising affinity for an antigen on a target cell.

Embodiment 39 provides a method of treating a disease or disorder in a subject in need thereof, comprising administering to the subject a modified T cell comprising: a modification in an endogenous gene locus encoding PRDM1, wherein the modification is capable of downregulating gene expression of, or knocking out, endogenous PRDM1; a modification in an endogenous gene locus encoding NR4A3, wherein the modification is capable of downregulating gene expression of, or knocking out, endogenous NR4A3; and an exogenous T cell receptor (TCR) and/or chimeric antigen receptor (CAR) comprising affinity for an antigen on a target cell.

Embodiment 40 provides the method of treating a disease or disorder in a subject in need thereof, comprising administering to the subject a modified T cell comprising: a modification in an endogenous gene locus encoding PRDM1, wherein the modification is capable of downregulating gene expression of, or knocking out, endogenous PRDM1; a modification in an endogenous gene locus encoding TGFβRII, wherein the modification is capable of downregulating gene expression of, or knocking out, endogenous TGFβRII; and an exogenous T cell receptor (TCR) and/or chimeric antigen receptor (CAR) comprising affinity for an antigen on a target cell.

Embodiment 41 provides the method of any of embodiments 37-40, wherein the antigen on a target cell is a tumor associated antigen (TAA).

Embodiment 42 provides the method of any of embodiments 37-41, wherein the disease or disorder is cancer.

Embodiment 43 provides the method of any of embodiments 37-42, wherein the modified T cell is a gamma delta T cell.

Embodiment 44 provides the method of any of embodiments 37-43, wherein the modified T cell is autologous.

Embodiment 45 provides the method of any of embodiments 37-44, wherein the subject is a human.

### OTHER EMBODIMENTS

The recitation of a listing of elements in any definition of a variable herein includes definitions of that variable as any single element or combinations (or subcombinations) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variation.

1. A modified immune cell or precursor cell thereof, comprising:
(a) modification in an endogenous gene locus encoding PRDM1, wherein the modification is capable of downregulating gene expression of, or knocking out, endogenous PRDM1;
a modification in an endogenous gene locus encoding NR4A3, wherein the modification is capable of downregulating gene expression of, or knocking out, endogenous NR4A3; and
an exogenous T cell receptor (TCR) and/or chimeric antigen receptor (CAR) comprising affinity for an antigen on a target cell;
(b) a modification in an endogenous gene locus encoding PRDM1, wherein the modification is capable of downregulating gene expression of, or knocking out, endogenous PRDM1; and
an exogenous T cell receptor (TCR) and/or chimeric antigen receptor (CAR) comprising affinity for an antigen on a target cell; or
(c) a modification in an endogenous gene locus encoding PRDM1, wherein the modification is capable of downregulating gene expression of, or knocking out, endogenous PRDM1;
a modification in an endogenous gene locus encoding TGFβRII, wherein the modification is capable of downregulating gene expression of, or knocking out, endogenous TGFβRII; and
an exogenous T cell receptor (TCR) and/or chimeric antigen receptor (CAR) comprising affinity for an antigen on a target cell.

2. (canceled)

3. (canceled)

4. The modified immune cell or precursor cell of claim 1, wherein the modification comprises a CRISPR-mediated modification.

5. The modified immune cell or precursor cell of claim 4, wherein the CRISPR-mediated modification is introduced by a CRISPR system comprising a guide RNA that comprises a guide sequence that is sufficiently complementary with a target sequence in the endogenous gene locus encoding PRDM1, NR4A3 or TGFβRII.

6. The modified immune cell or precursor cell of claim 5, wherein the guide RNA comprises a nucleic acid sequence set forth in SEQ ID NO: 2 or SEQ ID NO: 4.

7. The modified immune cell or precursor cell of claim 1, wherein the modification in the endogenous gene locus is selected from the group consisting of a substitution, an insertion, a deletion, and an insertion/deletion.

8. The modified immune cell or precursor cell of claim 1, wherein the CAR comprises an antigen binding domain, a transmembrane domain, and an intracellular domain.

9. The modified immune cell or precursor cell of claim 8, wherein the antigen binding domain is capable of binding a tumor associated antigen (TAA).

10. The modified immune cell or precursor cell of claim 8, wherein the antigen binding domain is selected from the group consisting of an antibody, an scFv, and a Fab.

11. The modified immune cell or precursor cell of claim 8, wherein the transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

12. The modified immune cell or precursor cell of claim 1, wherein the CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

13. The modified immune cell or precursor cell of claim 1, wherein the CAR comprises an intracellular domain comprising an intracellular domain selected from the group consisting of cytoplasmic signaling domains of a human CD3 zeta chain, FcγRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

14. The modified immune cell or precursor cell of claim 1, wherein the antigen on a target cell is a tumor associated antigen (TAA).

15. The modified immune cell or precursor cell of claim 1, wherein the modified cell is resistant to cell exhaustion.

16. The modified immune cell or precursor cell of claim 1, wherein the modified cell is an autologous cell.

17. The modified immune cell or precursor cell of claim 1, wherein the modified cell is a cell isolated from a human subject.

18. (canceled)

19. The modified immune cell or precursor cell of claim 1, wherein the modified cell is a modified T cell.

20. The modified immune cell or precursor cell of claim 1, wherein the modified cell is a gamma delta T cell.

21. The modified immune cell or precursor cell of claim 1, wherein the modified cell is a modified T cell resistant to T cell exhaustion.

22. A method of treating cancer in a subject in need thereof, the method comprising administering to the subject a composition comprising the modified immune cell or precursor cell thereof of claim 1.

23. A method for generating a modified immune cell or precursor cell thereof, comprising:
(a) introducing into an immune or precursor cell a CRISPR system comprising one or more polypeptides and/or nucleic acids capable of downregulating gene expression of endogenous PRDM1; and
introducing into the immune or precursor cell a nucleic acid encoding an exogenous CAR and/or TCR, wherein the exogenous CAR and/or TCR comprises affinity for an antigen on a target cell;
(b) introducing into an immune or precursor cell a CRISPR system comprising one or more polypeptides and/or nucleic acids capable of downregulating gene expression of endogenous PRDM1;
introducing into an immune or precursor cell a CRISPR system comprising one or more polypeptides and/or nucleic acids capable of downregulating gene expression of endogenous NR4A3; and
introducing into the immune or precursor cell a nucleic acid encoding an exogenous CAR and/or TCR, wherein the exogenous CAR and/or TCR comprises affinity for an antigen on a target cell: or
(c) introducing into an immune or precursor cell a CRISPR system comprising one or more polypeptides and/or nucleic acids capable of downregulating gene expression of endogenous PRDM1;
introducing into an immune or precursor cell a CRISPR system comprising one or more polypeptides and/or nucleic acids capable of downregulating gene expression of endogenous NR4A3; and
introducing into the immune or precursor cell a nucleic acid encoding an exogenous CAR and/or TCR, wherein the exogenous CAR and/or TCR comprises affinity for an antigen on a target cell.

24. (canceled)

25. (canceled)

26. The method of any of claim 23, wherein the one or more polypeptides and/or nucleic acids capable of downregulating gene expression of endogenous PRDM1 introduces a CRISPR-mediated modification in an endogenous gene locus encoding PRDM1, and/or the one or more polypeptides and/or nucleic acids capable of downregulating gene expression of endogenous NR4A3 introduces a CRISPR-mediated modification in an endogenous gene locus encoding NR4A3, and/or the one or more polypeptides and/or nucleic acids capable of downregulating gene expression of endogenous TGFβRII introduces a CRISPR-mediated modification in an endogenous gene locus encoding TGFβRII.

27. The method of claim 26, wherein the CRISPR-mediated modification is selected from the group consisting of a substitution, an insertion, a deletion, and an insertion/deletion.

28. The method of any of claim 23, wherein the CRISPR system comprises a CRISPR nuclease and a guide RNA.

29. The method of claim 28, wherein the CRISPR nuclease is Cas9.

30. The method of claim 28, wherein the CRISPR nuclease and the guide RNA comprise a ribonucleoprotein (RNP) complex.

31. The method of claim 30, wherein the RNP complex is introduced by electroporation.

32. The method of claim 28, wherein the guide RNA comprises a nucleic acid sequence set forth in SEQ ID NO: 2 or SEQ ID NO: 4.

33. The method of any of claim 23, wherein the nucleic acid encoding an exogenous CAR and/or TCR is introduced via viral transduction.

34. The method of claim 33 wherein the viral transduction comprises contacting the immune or precursor cell with a viral vector comprising the nucleic acid encoding an exogenous CAR and/or TCR.

35. The method of claim 34, wherein the viral vector is selected from the group consisting of a retroviral vector, a lentiviral vector, an adenoviral vector, and an adeno-associated viral vector.

36. The method of claim 35, wherein the viral vector is a lentiviral vector.

37. A method of treating cancer in a subject in need thereof, comprising administering to the subject modified immune or precursor cell generated by the method of claim 23.

38. A method of treating a disease or disorder in a subject in need thereof, comprising administering to the subject a modified T cell comprising:
(a) modification in an endogenous gene locus encoding PRDM1, wherein the modification is capable of downregulating gene expression of, or knocking out, endogenous PRDM1; and
an exogenous T cell receptor (TCR) and/or chimeric antigen receptor (CAR) comprising affinity for an antigen on a target cell,
(b) a modification in an endogenous gene locus encoding PRDM1, wherein the modification is capable of downregulating gene expression of, or knocking out, endogenous PRDM1;
a modification in an endogenous gene locus encoding NR4A3, wherein the modification is capable of downregulating gene expression of, or knocking out, endogenous NR4A3; and
an exogenous T cell receptor (TCR) and/or chimeric antigen receptor (CAR) comprising affinity for an antigen on a target cell: or
(c) a modification in an endogenous gene locus encoding PRDM1, wherein the modification is capable of downregulating gene expression of, or knocking out, endogenous PRDM1;
a modification in an endogenous gene locus encoding TGFβRII, wherein the modification is capable of downregulating gene expression of, or knocking out, endogenous TGFβRII; and
an exogenous T cell receptor (TCR) and/or chimeric antigen receptor (CAR) comprising affinity for an antigen on a target cell.

39. (canceled)

40. (canceled)

41. The method of claim 37, wherein the antigen on a target cell is a tumor associated antigen (TAA).

42. The method of any of claim 37, wherein the disease or disorder is cancer.

43. The method of any of claim 37, wherein the modified T cell is a gamma delta T cell.

44. The method of any of claim 37, wherein the modified T cell is autologous.

45. The method of any of claim 37, wherein the subject is a human.


SEQUENCE LISTING (33 sequences)

SEQ ID NO: 1 - DNA, 20 bp, synthetic construct
  source 1..20: mol_type = other DNA
ccatcgtaagcaaaccttag

SEQ ID NO: 2 - DNA, 20 bp, synthetic construct
  source 1..20: mol_type = other DNA
catcagcaccagaatcccag

SEQ ID NO: 3 - DNA, 20 bp, synthetic construct
  source 1..20: mol_type = other DNA
tcagggagtagaagccagag

SEQ ID NO: 4 - DNA, 20 bp, synthetic construct
  source 1..20: mol_type = other DNA
ccttggcagcactgagatca

SEQ ID NO: 5 - DNA, 24 bp, synthetic construct
  source 1..24: mol_type = other DNA
tctcagaaggagccacaggaacgg

SEQ ID NO: 6 - DNA, 24 bp, synthetic construct
  source 1..24: mol_type = other DNA
cacccaccctatgctgcaagttgc

SEQ ID NO: 7 - DNA, 30 bp, synthetic construct
  source 1..30: mol_type = other DNA
gaggagaggatgacacttcctctctgtttc

SEQ ID NO: 8 - DNA, 30 bp, synthetic construct
  source 1..30: mol_type = other DNA
ctgcccagcacctccatgtacttcaagcag

SEQ ID NO: 9 - protein, 5 aa, synthetic construct
  REPEAT 1..5: note = repeat n times, where n is an integer of 1
GSGGS

SEQ ID NO: 10 - protein, 4 aa, synthetic construct
  REPEAT 1..4: note = repeat n times, where n is an integer of 1
GGGS

SEQ ID NO: 11 - protein, 5 aa, synthetic construct
  REPEAT 1..5: note = repeat n times, where n is an integer of 1
GGGGS

SEQ ID NO: 12 - protein, 4 aa, synthetic construct
GGSG

SEQ ID NO: 13 - protein, 5 aa, synthetic construct
GGSGG

SEQ ID NO: 14 - protein, 5 aa, synthetic construct
GSGSG

SEQ ID NO: 15 - protein, 5 aa, synthetic construct
GSGGG

SEQ ID NO: 16 - protein, 5 aa, synthetic construct
GGGSG

SEQ ID NO: 17 - protein, 5 aa, synthetic construct
GSSSG

SEQ ID NO: 18 - protein, 5 aa, synthetic construct
GGGGS

SEQ ID NO: 19 - protein, 15 aa, synthetic construct
GGGGSGGGGSGGGGS

SEQ ID NO: 20 - DNA, 45 bp, synthetic construct
  source 1..45: mol_type = other DNA
ggtggcggtggctcgggcggtggtgggtcgggtggcggcggatct

SEQ ID NO: 21 - protein, 5 aa, synthetic construct
DKTHT

SEQ ID NO: 22 - protein, 4 aa, synthetic construct
CPPC

SEQ ID NO: 23 - protein, 15 aa, synthetic construct
CPEPKSCDTPPPCPR

SEQ ID NO: 24 - protein, 12 aa, synthetic construct
ELKTPLGDTTHT

SEQ ID NO: 25 - protein, 10 aa, synthetic construct
KSCDKTHTCP

SEQ ID NO: 26 - protein, 7 aa, synthetic construct
KCCVDCP

SEQ ID NO: 27 - protein, 7 aa, synthetic construct
KYGPPCP

SEQ ID NO: 28 - protein, 15 aa, synthetic construct
EPKSCDKTHTCPPCP

SEQ ID NO: 29 - protein, 12 aa, synthetic construct
ERKCCVECPPCP

SEQ ID NO: 30 - protein, 17 aa, synthetic construct
ELKTPLGDTTHTCPRCP

SEQ ID NO: 31 - protein, 12 aa, synthetic construct
SPNMVPHAHHAQ

SEQ ID NO: 32 - protein, 15 aa, synthetic construct
EPKSCDKTYTCPPCP

SEQ ID NO: 33 - protein, 9 aa, synthetic construct
SLLMWITQC